(19) United States
(12) Patent Application Publication
(10) Pub. No.: US 2025/0171413 A1
(43) Pub. Date: May 29, 2025

(54) ANTIVIRAL INDOLINYL COMPOUNDS AND USES THEREOF

(71) Applicant: Gilead Sciences, Inc., Foster City, CA (US)

(72) Inventors: Aesop CHO, Mountain View, CA (US); Thomas P. STRATTON, San Francisco, CA (US)

(21) Appl. No.: 18/951,539

(22) Filed: Nov. 18, 2024

Related U.S. Application Data
(60) Provisional application No. 63/600,138, filed on Nov. 17, 2023.

(51) Int. Cl. C07D 401/10; A61K 31/4439; A61K 45/06; C07D 405/14

(52) U.S. Cl. CPC C07D 401/10; A61K 31/4439; A61K 45/06; C07D 405/14

(57) ABSTRACT

The present disclosure relates to indolinyl compounds. The present disclosure further relates to compounds that inhibit viral helicase-primase. The present disclosure further relates to the use of the compounds for the preparation a medicament for the treatment of diseases and/or condition through inhibiting viral helicase-primase. The present disclosure also relates to use of those compounds in the treatment of viral infections. The present disclosure further relates to intermediates for its preparation and to pharmaceutical compositions containing those compounds.

### CROSS-REFERENCE TO RELATED APPLICATIONS

This application claims priority to U.S. Provisional Application No. 63/600,138, filed Nov. 17, 2023, which is incorporated by reference in its entirety.

### FIELD

The present disclosure relates to indolinyl compounds. The present disclosure also relates to use of those compounds in the treatment of viral infections. The present disclosure further relates to intermediates for its preparation and to pharmaceutical compositions containing those compounds.

### BACKGROUND

Herpesviruses have a very high global prevalence and disease burden. Herpesvirues include herpes simplex virus 1 (HSV-1), herpes simplex virus 2 (HSV-2), varicella-zoster virus, Epstein-Barr virus, and cytomegalovirus. HSV-1 and HSV-2 are contagious human pathogens. HSV-1 is mainly transmitted by oral-to-oral contact to cause oral herpes, while HSV-2 is a sexually transmitted infection that causes genital herpes. These infections are lifelong and characterized by periodic re-activation and viral shedding, which can cause symptoms such as painful blisters or ulcers and transmissions to others. Currently available medications to treat these infections are largely based on nucleoside analogs such as acyclovir, famciclovir, and valacyclovir. Although effective at reducing severity and frequency of the symptoms, these compounds do not eliminate viral shedding and thus a risk of transmission. In addition, dosing regimens are complex and inconvenient.

Herpesvirus encodes its own helicase and primase for synthesis of viral DNA. The helicase-primase complex performs a key role in viral DNA replication. The helicase separates the viral DNA double helix, and the primase synthesizes RNA primers on the single-stranded DNA which initiates DNA synthesis directed by the DNA polymerase (Kleymann G. 2004).

There is a need for new agents that are more effective and safer with improved pharmacokinetics. Inhibition of viral helicase-primase interferes with viral replication and thus could lead to the development of drugs with desirable selectivity, potency, metabolic stability, or reduced detrimental effects.

### SUMMARY

The present disclosure relates to indolinyl compounds. The present disclosure also relates to compounds that inhibit viral helicase-primase. The present disclosure further relates to use of the compounds for the treatment and/or prevention of diseases and/or conditions.

In one embodiment, provided herein is a compound of Formula (I),

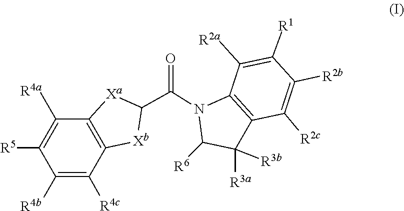

or a pharmaceutically acceptable salt thereof, wherein

R¹ is

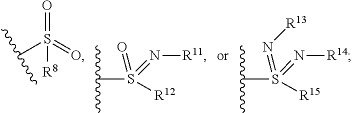

R^{2a}, R^{2b}, and R^{2c} are each independently H, halogen, C₁₋₆ alkyl, C₁₋₆ haloalkyl, C₁₋₆ alkoxy, —CN, C₁₋₆haloalkoxy, or —SCF₃;
R^{3a} and R^{3b} are each independently H, halogen, C₁₋₆ alkyl, or C₁₋₆ haloalkyl;
X^{a} and X^{b} are each independently O or CR^{7a}R^{7b};
each R^{7a} is independently H, C₁₋₆ alkyl, or C₁₋₆ haloalkyl;
each R^{7b} is independently H, C₁₋₆ alkyl, or C₁₋₆ haloalkyl;
R^{4a}, R^{4b}, and R^{4c} are each independently H, halogen, C₁₋₆ alkyl, C₁₋₆ haloalkyl, C₁₋₆ alkoxy, —CN, —SCF₃, or C₃₋₆ cycloalkyl optionally substituted with Z⁴;
R⁵ is C₆₋₁₀ aryl or heteroaryl; wherein the aryl or heteroaryl of R⁵ is optionally substituted with one to three Z⁵, which may be the same or different; the heteroaryl of R⁵ is 5 to 10 membered heteroaryl having one to three heteroatoms each independently N, O, or S;
R⁶ is H, C₁₋₆ alkyl, C₁₋₆ haloalkyl, C₂₋₈ alkoxyalkyl, or C₃₋₆ cycloalkyl, wherein the cycloalkyl of R⁶ is optionally substituted with one to three Z⁶;
R⁸ is NHR⁹ or NR⁹R¹⁰;
each R⁹, R¹⁰, and R¹² is independently C₁₋₆ alkyl, C₁₋₆ haloalkyl, —COR^{9a}, or C₃₋₆ cycloalkyl; the alkyl or cycloalkyl of R⁹, R¹⁰, or R¹² is optionally substituted with one to three groups independently selected from —OR^{12a}, and —SR^{12a};
R¹¹ is H, C₁₋₆ alkyl, C₁₋₆ haloalkyl, —COR^{9a}, or C₃₋₆ cycloalkyl; the alkyl or cycloalkyl of R¹¹ is optionally substituted with one to three groups each independently —OR^{12a} or —SR^{12a}; each R¹³ and R¹⁴ is independently H or C₁₋₆ alkyl;
R¹⁵ is C₁₋₆ alkyl, C₁₋₆ haloalkyl, or C₃₋₆ cycloalkyl; the alkyl or cycloalkyl of R¹⁵ is optionally substituted with one to three groups each independently —OR^{12a} or —SR^{12a};
each R^{9a} and R^{12a} is independently C₁₋₆ alkyl; and
each Z⁴, Z⁵, and Z⁶ is independently halogen, —CN, C₁₋₆ alkyl, C₁₋₆ haloalkyl, C₁₋₆ alkoxy, or C₁₋₆haloalkoxy;
provided that when R¹¹ is H, R¹² is not —CH₃.

In some embodiments, provided herein are pharmaceutical compositions comprising a compound provided herein, or pharmaceutically acceptable salt thereof, and a pharmaceutically acceptable excipient or carrier. In some embodiments, the pharmaceutical compositions comprise a therapeutically effective amount of a compound provided herein, or pharmaceutically acceptable salt thereof, and a pharmaceutically acceptable excipient or carrier.

In some embodiments, the pharmaceutical compositions provided herein further comprise one or more (e.g., one, two, three, four, one or two, one to three, or one to four) additional therapeutic agents, or pharmaceutically acceptable salts thereof. In some embodiments, the pharmaceutical compositions further comprise a therapeutically effective amount of the one or more (e.g., one, two, three, four, one or two, one to three, or one to four) additional therapeutic agents, or pharmaceutically acceptable salts thereof.

In some embodiments, the present disclosure provides methods of inhibiting the helicase-primase of herpesviruses in a subject in need thereof, comprising administering to the subject a therapeutically effective amount of a compound provided herein (e.g., a compound of Formula (I), (Ia), (II), (IIa), (III), (IIIa), (IIIb), (IIIc), (IIId), or (IIIe), or pharmaceutically acceptable salt thereof, or a pharmaceutical composition provided herein.

In some embodiments, the present disclosure provides methods of treating a patient having a herpesvirus mediated condition, comprising administering to the patient a therapeutically effective amount of a compound provided herein (e.g., a compound of Formula (I), (Ia), (II), (IIa), (III), (IIIa), (IIIb), (IIIc), (IIId), or (IIIe)), or pharmaceutically acceptable salt thereof, or a pharmaceutical composition provided herein.

### DETAILED DESCRIPTION

The present disclosure relates to compounds that inhibit the helicase primase of herpesviruses. In one embodiment, the present disclosure relates to indolinyl compounds. The present disclosure further relates to use of the compounds for the treatment and/or prevention of diseases and/or conditions by said compounds. The disclosure also relates to compositions and methods of treating and/or preventing viral infections that include an inhibitor of herpesvirus helicase primase in combination with one or more additional therapeutic agents.

Patients infected with herpesviruses can benefit from the treatment with an inhibitor of herpesvirus helicase primase and optionally one or more additional therapeutic agents.

### Definitions and General Parameters

The description below is made with the understanding that the present disclosure is to be considered as an exemplification of the claimed subject matter and is not intended to limit the appended claims to the specific embodiments illustrated. The headings used throughout this disclosure are provided for convenience and are not to be construed to limit the claims in any way. Embodiments illustrated under any heading may be combined with embodiments illustrated under any other heading.

Unless defined otherwise, all technical and scientific terms used herein have the same meaning as commonly understood by one of ordinary skill in the art. It must be noted that as used herein and in the appended claims, the singular forms “a”, “and”, and “the” include plural referents unless the context clearly dictates otherwise. Thus, e.g., reference to “the compound” includes a plurality of such compounds and reference to “the assay” includes reference to one or more assays and equivalents thereof known to those skilled in the art, and so forth.

As used in the present specification, the following terms and phrases are generally intended to have the meanings as set forth below, except to the extent that the context in which they are used indicates otherwise.

A dash (“—”) that is not between two letters or symbols is used to indicate a point of attachment for a substituent. For example, —CONH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named. A solid line coming out of the center of a ring indicates that the point of attachment for a substituent on the ring can be at any ring atom. For example, R^{a} in the structure below can be attached to any of the five carbon ring atoms or R^{a} can replace the hydrogen attached to the nitrogen ring atom:

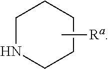

The prefix “Cᵤ₋ᵥ” indicates that the following group has from u to v carbon atoms. For example, “C₁₋₆ alkyl” indicates that the alkyl group has from 1 to 6 carbon atoms. Likewise, the term “x-y membered” rings, wherein x and y are numerical ranges, such as “3 to 12-membered heterocyclyl”, refers to a ring containing x-y atoms (e.g., 3-12), of which up to 80% may be heteroatoms, such as N, O, S, P, and the remaining atoms are carbon.

Also, certain commonly used alternative chemical names may or may not be used. For example, a divalent group such as a divalent “alkyl” group, a divalent “aryl” group, etc., may also be referred to as an “alkylene” group or an “alkylenyl” group, or alkylyl group, an “arylene” group or an “arylenyl” group, or arylyl group, respectively.

“A compound disclosed herein” or “a compound of the present disclosure” or “a compound provided herein” or “a compound described herein” refers to the compounds of Formula (I), (Ia), (II), (IIa), (III), (IIIa), (IIIb), (IIIc), (IIId), or (IIIe). Also included are the specific compounds provided herein.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), 1 to 4 carbon atoms (i.e., C₁₋₄ alkyl), or 1 to 3 carbon atoms (i.e., C₁₋₃ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

“Alkenyl” refers to an aliphatic group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Alkynyl” refers to an aliphatic group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Acyl” refers to a group —C(═O)R, wherein R is hydrogen, alkyl, cycloalkyl, heterocyclyl, aryl, heteroalkyl, or heteroaryl; each of which may be optionally substituted, as defined herein. Examples of acyl include formyl, acetyl, cylcohexylcarbonyl, cyclohexylmethyl-carbonyl, and benzoyl.

“Alkoxy” refers to an alkyl group having an oxygen atom that connects the alkyl group to the point of attachment: alkyl-O—. As for alkyl group, alkoxy groups will have any suitable number of carbon atoms, such as C₁₆. Alkoxy groups include, for example, methoxy, ethoxy, propoxy, isopropoxy, butoxy, 2butoxy, isobutoxy, secbutoxy, tertbutoxy, pentoxy, hexoxy, etc. The alkoxy groups can be further substituted with a variety of substituents described within. Alkoxy groups can be substituted or unsubstituted.

“Alkoxyalkyl” refers an alkoxy group linked to an alkyl group which is linked to the remainder of the compound. Alkoxyalkyl has any suitable number of carbon atoms, such as from 2 to 6 (C₂₋₆ alkoxyalkyl), 2 to 5 (C₂₋₅ alkoxyalkyl), 2 to 4 (C₂₋₄ alkoxyalkyl), or 2 to 3 (C₂₋₃ alkoxyalkyl). The number of carbons refers to the total number of carbons in the alkoxy and the alkyl group. For example, in some embodiments, C₆ alkoxyalkyl refers to ethoxy (C₂ alkoxy) linked to a butyl (C₄ alkyl), and in other embodiments, n-propoxy (C₃ alkoxy) linked to isopropyl (C₃ alkyl). Alkoxy and alkyl are as defined above where the alkyl is divalent, and can include, but is not limited to, methoxymethyl (CH₃OCH₂), methoxyethyl (CH₃OCH₂CH₂) and others.

“Amino” refers to the group NR^{y}R^{z} wherein R^{y} and R^{z} are independently selected from the group consisting of hydrogen, alkyl, haloalkyl, aryl, heteroaryl, cycloalkyl, or heterocyclyl; each of which may be optionally substituted.

“Aryl” as used herein refers to a single all carbon aromatic ring or a multicyclic all carbon ring system wherein at least one of the rings is aromatic. For example, in some embodiments, an aryl group has 6 to 20 carbon atoms, 6 to 14 carbon atoms, or 6 to 12 carbon atoms. Aryl includes a phenyl radical. Aryl also includes multicyclic ring systems (e.g., ring systems comprising 2, 3 or 4 rings) having 9 to 20 carbon atoms, e.g., 9 to 16 carbon atoms, in which at least one ring is aromatic and wherein the other rings may be aromatic or not aromatic (i.e., carbocycle). Such multicyclic ring systems are optionally substituted with one or more (e.g., 1, 2 or 3) oxo groups on any carbocycle portion of the multicyclic ring system. The rings of the multicyclic ring system can be connected to each other via fused, spiro and bridged bonds when allowed by valency requirements. It is also to be understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl and 1,2,3,4-tetrahydronaphthyl. Non-limiting examples of aryl groups include, but are not limited to, phenyl, indenyl, naphthyl, 1,2,3,4-tetrahydronaphthyl, anthracenyl, and the like.

“Cyano” or “carbonitrile” refers to the group CN.

“Cycloalkyl” refers to a saturated or partially saturated cyclic alkyl group having a single ring or multiple rings including fused, bridged, and spiro ring systems. The term “cycloalkyl” includes cycloalkenyl groups (i.e., the cyclic group having at least one double bond). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl.

“Fused” refers to a ring which is bound to an adjacent ring. In some embodiments the fused ring system is a heterocyclyl. In some embodiments the fused ring system is a oxabicyclohexanyl. In some embodiments the fused ring system is

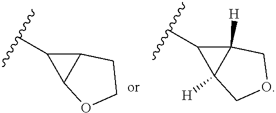 [SMILES: *C1C2CCOC12 ; *C1[C@@H]2COC[C@@H]12]

“Bridged” refers to a ring fusion wherein non-adjacent atoms on a ring are joined by a divalent substituent, such as alkylenyl group, an alkylenyl group containing one or two heteroatoms, or a single heteroatom. Quinuclidinyl and admantanyl are examples of bridged ring systems. In some embodiments the bridged ring is a bicyclopentyl (e.g., bicyclo[1.1.1]pentyl), bicycloheptyl (e.g., bicyclo[2.2.1]heptyl, bicyclo[3.1.1]heptyl), or bicyclooctyl (e.g., bicyclo[2.2.2]octyl). In some embodiments, the bridged ring

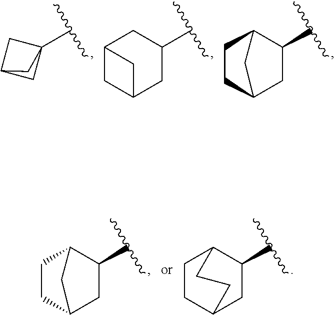

“Spiro” refers to a ring substituent which is joined by two bonds at the same carbon atom. Examples of spiro groups include 1,1-diethylcyclopentane, dimethyl-dioxolane, and 4-benzyl-4-methylpiperidine, wherein the cyclopentane and piperidine, respectively, are the spiro substituents. In some embodiments the spiro substituent is a spiropentanyl (spiro[a.b]pentanyl), spirohexanyl, spiroheptanyl, spirooctyl (e.g., spiro[2.5]octyl), spirononanyl (e.g., spiro[3.5]nonanyl), spirodecanyl (e.g., spiro[4.5]decanyl), or spiroundecanyl (e.g., spiro[5.5]undecanyl). In some embodiments the spiro substituent is

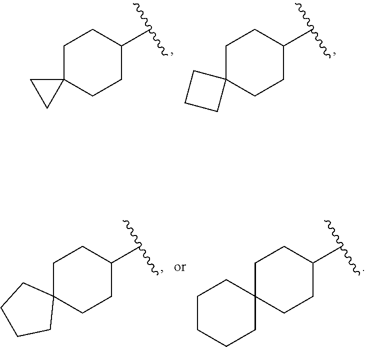 [SMILES: *C1CCC2(CC1)CC2 ; *C1CCC2(CCC2)CC1 ; *C1CCC2(CCCC2)CC1 ; *C1CCC2(CCCCC2)CC1]

“Halogen” or “halo” includes fluoro, chloro, bromo, and iodo.

“Haloalkyl” as used herein refers to an alkyl as defined herein, wherein one or more hydrogen atoms of the alkyl are independently replaced by a halo substituent, which may be the same or different. For example, C₁₋₄ haloalkyl is a C₁₋₄ alkyl wherein one or more of the hydrogen atoms of the C₁₋₄ alkyl have been replaced by a halo substituent. Examples of haloalkyl groups include but are not limited to fluoromethyl, fluorochloromethyl, difluoromethyl, difluorochloromethyl, trifluoromethyl, 1,1,1-trifluoroethyl and pentafluoroethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₆. The alkoxy groups can be substituted with 1, 2, 3, or more halogens. When all the hydrogens are replaced with a halogen, for example by fluorine, the compounds are persubstituted, for example, perfluorinated. Haloalkoxy includes, but is not limited to, trifluoromethoxy, 2,2,2, trifluoroethoxy, perfluoroethoxy, etc.

The term “heteroaryl” as used herein refers to a single aromatic ring or a multicyclic ring. The term includes single aromatic rings of from about 1 to 6 carbon atoms and about 1-4 heteroatoms selected from the group consisting of oxygen, nitrogen and sulfur in the rings. The sulfur and nitrogen atoms may also be present in an oxidized form provided the ring is aromatic. Such rings include but are not limited to pyridyl, pyrimidinyl, oxazolyl or furyl. The term also includes multicyclic ring systems (e.g., ring systems comprising 2 or 3 rings) wherein a heteroaryl group, as defined above, can be fused with one or more heteroaryls (e.g. naphthyridinyl), carbocycles (e.g., 5,6,7,8-tetrahydroquinolyl) or aryls (e.g., indazolyl) to form a multicyclic ring. Such multicyclic rings may be optionally substituted with one or more (e.g., 1, 2 or 3) oxo groups on the carbocycle portions of the multicyclic ring. It is to be understood that the point of attachment of a heteroaryl multicyclic ring, as defined above, can be at any position of the ring including a heteroaryl, aryl or a carbocycle portion of the ring. Exemplary heteroaryls include but are not limited to pyridyl, pyrrolyl, pyrazinyl, pyrimidinyl, pyridazinyl, pyrazolyl, thienyl, indolyl, imidazolyl, oxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl and thianaphthenyl.

“Heterocyclyl” or “heterocyclic ring” or “heterocycle” as used herein refers to a single saturated or partially unsaturated ring or a multicyclic ring. The term includes single saturated or partially unsaturated ring (e.g., 3, 4, 5, 6 or 7-membered ring) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Such rings include but are not limited to azetidinyl, tetrahydrofuranyl or piperidinyl. The term also includes multicyclic ring systems (e.g., ring systems comprising 2 or 3 rings) wherein a heterocycle group (as defined above) can be connected to two adjacent atoms (fused heterocycle) with one or more heterocycles (e.g., decahydronapthyridinyl), heteroaryls (e.g., 1,2,3,4-tetrahydronaphthyridinyl), carbocycles (e.g., decahydroquinolyl) or aryls. It is to be understood that the point of attachment of a heterocycle multicyclic ring, as defined above, can be at any position of the ring including a heterocyle, heteroaryl, aryl or a carbocycle portion of the ring. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl and 1,4-benzodioxanyl. Exemplary fused bicyclic heterocycles include, but are not limited to

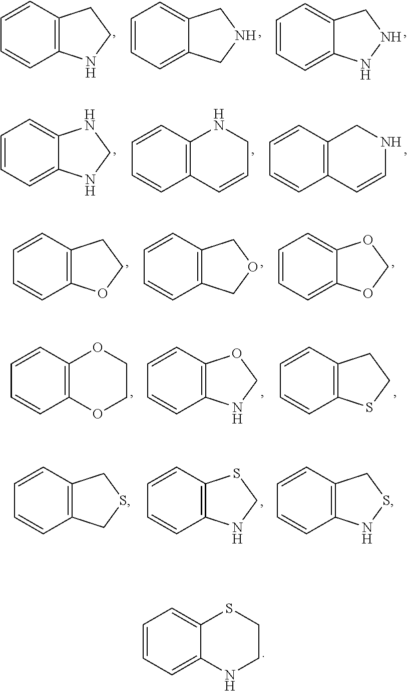

“Hydroxy” or “hydroxyl” refers to the group OH.

“Oxo” refers to the group (═O) or (O).

“Sulfonyl” refers to the group S(O)₂R^{c}, where R^{c} is alkyl, heterocyclyl, cycloalkyl, heteroaryl, or aryl. Examples of sulfonyl are methylsulfonyl, ethylsulfonyl, phenylsulfonyl, and toluenesulfonyl.

Whenever the graphical representation of a group terminates in a singly bonded nitrogen atom, that group represents an —NH₂ group unless otherwise indicated. Similarly, unless otherwise expressed, hydrogen atom(s) are implied and deemed present where necessary in view of the knowledge of one of skill in the art to complete valency or provide stability.

The terms “optional” or “optionally” mean that the subsequently described event or circumstance may or may not occur, and that the description includes instances where said event or circumstance occurs and instances in which it does not. Also, the term “optionally substituted” means that any one or more hydrogen atoms on the designated atom or group may or may not be replaced by a moiety other than hydrogen.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to [(substituted aryl) substituted aryl] substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. For example, the term “substituted aryl” includes, but is not limited to, “alkylaryl.” Unless specified otherwise, where a group is described as optionally substituted, any substituents of the group are themselves unsubstituted.

In some embodiments, the term “substituted alkyl” refers to an alkyl group having one or more substituents including hydroxyl, halo, amino, alkoxy, cycloalkyl, heterocyclyl, aryl, and heteroaryl. In additional embodiments, “substituted cycloalkyl” refers to a cycloalkyl group having one or more substituents including alkyl, haloalkyl, cycloalkyl, heterocyclyl, aryl, heteroaryl, amino, alkoxy, halo, oxo, and hydroxyl; “substituted heterocyclyl” refers to a heterocyclyl group having one or more substituents including alkyl, amino, haloalkyl, heterocyclyl, cycloalkyl, aryl, heteroaryl, alkoxy, halo, oxo, and hydroxyl; “substituted aryl” refers to an aryl group having one or more substituents including halo, alkyl, amino, haloalkyl, cycloalkyl, heterocyclyl, heteroaryl, alkoxy, and cyano; “substituted heteroaryl” refers to an heteroaryl group having one or more substituents including halo, amino, alkyl, haloalkyl, cycloalkyl, aryl, heterocyclyl, heteroaryl, alkoxy, and cyano and “substituted sulfonyl” refers to a group —S(O)₂R, in which R is substituted with one or more substituents including alkyl, cycloalkyl, heterocyclyl, aryl, and heteroaryl. In other embodiments, the one or more substituents may be further substituted with halo, alkyl, haloalkyl, hydroxyl, alkoxy, cycloalkyl, heterocyclyl, aryl, or heteroaryl, each of which is substituted. In other embodiments, the substituents may be further substituted with halo, alkyl, haloalkyl, alkoxy, hydroxyl, cycloalkyl, heterocyclyl, aryl, or heteroaryl, each of which is unsubstituted.

In some embodiments, a substituted cycloalkyl, a substituted heterocyclyl, a substituted aryl, and/or a substituted heteroaryl includes a cycloalkyl, a heterocyclyl, an aryl, and/or a heteroaryl that has a substituent on the ring atom to which the cycloalkyl, heterocyclyl, aryl, and/or heteroaryl is attached to the rest of the compound. For example, in the moiety below, the cyclopropyl is substituted with a methyl group:

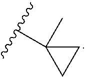 [SMILES: *C1(C)CC1]

The disclosures illustratively described herein may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

The compounds of the present disclosure can be in the form of a pharmaceutically acceptable salt. The term “pharmaceutically acceptable salts” refers to salts prepared from pharmaceutically acceptable non-toxic bases or acids, including inorganic bases or acids and organic bases or acids. In case the compounds of the present disclosure contain one or more acidic or basic groups, the disclosure also comprises their corresponding pharmaceutically or toxicologically acceptable salts or their pharmaceutically utilizable salts. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine, amino acids, or other bases known to persons skilled in the art. The compounds of the present disclosure which contain one or more basic groups, i.e., groups which can be protonated, can be present and can be used according to the disclosure in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to persons skilled in the art.

If the compounds of the present disclosure simultaneously contain acidic and basic groups in the molecule, the disclosure also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts.

The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts. Acids and bases useful for reaction with an underlying compound to form pharmaceutically acceptable salts (acid addition or base addition salts respectively) are known to one of skill in the art. Similarly, methods of preparing pharmaceutically acceptable salts from an underlying compound (upon disclosure) are known to one of skill in the art and are disclosed in for example, Berge, at al. Journal of Pharmaceutical Science, January 1977 vol. 66, No. 1, and other sources.

Furthermore, compounds disclosed herein may be subject to tautomerism. Where tautomerism, e.g., keto-enol tautomerism, of compounds or their prodrugs may occur, the individual forms, like, e.g., the keto and enol form, are each within the scope of the disclosure as well as their mixtures in any ratio. The same applies for stereoisomers, like, e.g., enantiomers, cis/trans isomers, diastereomers, conformers, and the like.

The term “protecting group” refers to a moiety of a compound that masks or alters the properties of a functional group or the properties of the compound as a whole. Chemical protecting groups and strategies for protection/deprotection are well known in the art (see e.g., Protective Groups in Organic Chemistry, Theodora W. Greene, John Wiley & Sons, Inc., New York, 1991). Protecting groups are often utilized to mask the reactivity of certain functional groups, to assist in the efficiency of desired chemical reactions, e.g., making and breaking chemical bonds in an ordered and planned fashion. The term “deprotecting” refers to removing the protecting group.

It will be appreciated by the skilled person that when lists of alternative substituents include members which, because of their valency requirements or other reasons, cannot be used to substitute a particular group, the list is intended to be read with the knowledge of the skilled person to include only those members of the list which are suitable for substituting the particular group.

Further the compounds of the present disclosure may be present in the form of solvates, such as those which include as solvate water, or pharmaceutically acceptable solvates, such as alcohols, in particular ethanol. A “solvate” is formed by the interaction of a solvent and a compound.

In certain embodiments, provided are optical isomers, racemates, or other mixtures thereof (e.g., scalemic mixtures) of the compounds described herein or a pharmaceutically acceptable salt or a mixture thereof. If desired, isomers can be separated by methods well known in the art, e.g., by liquid chromatography. In those situations, the single enantiomer or diastereomer, i.e., optically active form, can be obtained by asymmetric synthesis or by resolution. Resolution can be accomplished, for example, by conventional methods such as crystallization in the presence of a resolving agent, or chromatography, using for example, a chiral high-pressure liquid chromatography (HPLC) column.

A “stereoisomer” refers to a compound made up of the same atoms bonded by the same bonds but having different three-dimensional structures, which are not interchangeable. The present invention contemplates various stereoisomers and mixtures thereof and includes “enantiomers,” which refers to two stereoisomers whose molecules are non-superimposable mirror images of one another. “Diastereomers” are stereoisomers that have at least two asymmetric atoms, but which are not mirror-images of each other. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

Compounds disclosed herein and their pharmaceutically acceptable salts may, in some embodiments, include an asymmetric center and may thus give rise to enantiomers, diastereomers, and other stereoisomeric forms that may be defined, in terms of absolute stereochemistry, as (R)- or (S)- or, as (D)- or (L) for amino acids. Some embodiments include all such possible isomers-, as well as their racemic, scalemic, and optically pure forms. Optically active (+) and (−), (R)- and (S)-, or (D)- and (L) isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers-of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

Compositions provided herein that include a compound described herein or pharmaceutically acceptable salts, isomer, or a mixture thereof may include racemic mixtures, or mixtures containing an enantiomeric excess of one enantiomer or single diastereomers or diastereomeric mixtures. All such isomeric forms of these compounds are expressly included herein the same as if each and every isomeric form were specifically and individually listed.

Any formula or structure given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorus, fluorine, and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection, or imaging techniques, such as positron emission tomography (PET) or single-photon emission computed tomography (SPECT) including drug or substrate tissue distribution assays or in radioactive treatment of patients. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The disclosure also includes “deuterated analogs” of compounds disclosed herein, in which from 1 to n hydrogens attached to a carbon atom is/are replaced by deuterium, in which n is the number of hydrogens in the molecule. Such compounds may exhibit increased resistance to metabolism and thus be useful for increasing the half-life of any compound of Formula (I) when administered to a mammal, e.g., a human. See, e.g., Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have beneficial DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

Furthermore, the present disclosure provides pharmaceutical compositions comprising a compound of the present disclosure, or a prodrug compound thereof, or a pharmaceutically acceptable salt or solvate thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure can encompass any composition made by admixing at least one compound of the present disclosure and a pharmaceutically acceptable carrier.

As used herein, “pharmaceutically acceptable carrier” includes excipients or agents such as solvents, diluents, dispersion media, coatings, antibacterial and antifungal agents, isotonic and absorption delaying agents and the like that are not deleterious to the disclosed compound or use thereof. The use of such carriers and agents to prepare compositions of pharmaceutically active substances is well known in the art (see, e.g., Remington's Pharmaceutical Sciences, Mace Publishing Co., Philadelphia, PA 17th Ed. (1985); and Modern Pharmaceutics, Marcel Dekker, Inc. 3rd Ed. (G. S. Banker & C. T. Rhodes, Eds.).

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival. In some embodiments, the term “treatment” or “treating” means administering a compound or pharmaceutically acceptable salt of Formula (I), (Ia), (II), (IIa), (III), (IIIa), (IIIb), (IIIc), (IIId), or (IIIe) for the purpose of: (i) delaying the onset of a disease, that is, causing the clinical symptoms of the disease not to develop or delaying the development thereof; (ii) inhibiting the disease, that is, arresting the development of clinical symptoms; and/or (iii) relieving the disease, that is, causing the regression of clinical symptoms or the severity thereof.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In some embodiments, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition responsive to herpesvirus helicase-primase inhibitor. The therapeutically effective amount may vary depending on the subject, and disease or condition being treated, the weight and age of the subject, the severity of the disease or condition, and the manner of administering, which can readily be determined by one or ordinary skill in the art.

### Compounds

In one embodiment, the present disclosure provides a compound of Formula (I),

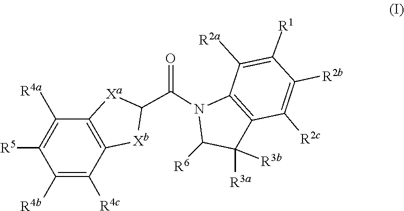

or a pharmaceutically acceptable salt thereof, wherein
R¹ is

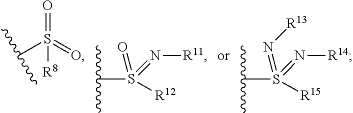

R^{2a}, R^{2b}, and R^{2c} are each independently H, halogen, C₁₋₆ alkyl, C₁₋₆ haloalkyl, C₁₋₆ alkoxy, —CN, C₁₋₆haloalkoxy, or —SCF₃;
R^{3a} and R^{3b} are each independently H, halogen, C₁₋₆ alkyl, or C₁₋₆ haloalkyl;
X^{a} and X^{b} are each independently O or CR^{7a}R^{7b};
each R^{7a} is independently H, C₁₋₆ alkyl, or C₁₋₆ haloalkyl;
each R^{7b} is independently H, C₁₋₆ alkyl, or C₁₋₆ haloalkyl;
R^{4a}, R^{4b}, and R^{4c} are each independently H, halogen, C₁₋₆ alkyl, C₁₋₆ haloalkyl, C₁₋₆ alkoxy, —CN, —SCF₃, or C₃₋₆ cycloalkyl optionally substituted with Z⁴;
R⁵ is C₆₋₁₀ aryl or heteroaryl; wherein the aryl or heteroaryl of R⁵ is optionally substituted with one to three Z⁵, which may be the same or different; the heteroaryl of R⁵ is 5 to 10 membered heteroaryl having one to three heteroatoms each independently N, O, or S;
R⁶ is H, C₁₋₆ alkyl, C₁₋₆ haloalkyl, C₂₋₈ alkoxyalkyl, or C₃₋₆ cycloalkyl, wherein the cycloalkyl of R⁶ is optionally substituted with one to three Z⁶;
R⁸ is NHR⁹ or NR⁹R¹⁰;
each R⁹, R¹⁰, and R¹² is independently C₁₋₆ alkyl, C₁₋₆ haloalkyl, —COR^{9a}, or C₃₋₆ cycloalkyl; the alkyl or cycloalkyl of R⁹, R¹⁰, or R¹² is optionally substituted with one to three groups independently selected from —OR^{12a}, and —SR^{12a};
R¹¹ is H, C₁₋₆ alkyl, C₁₋₆ haloalkyl, —COR^{9a}, or C₃₋₆ cycloalkyl; the alkyl or cycloalkyl of R¹¹ is optionally substituted with one to three groups each independently —OR^{12a} or —SR^{12a}; each R¹³ and R¹⁴ is independently H or C₁₋₆ alkyl;
R¹⁵ is C₁₋₆ alkyl, C₁₋₆ haloalkyl, or C₃₋₆ cycloalkyl; the alkyl or cycloalkyl of R¹⁵ is optionally substituted with one to three groups each independently —OR^{12a} or —SR^{12a};
each R^{9a} and R^{12a} is independently C₁₋₆ alkyl; and
each Z⁴, Z⁵, and Z⁶ is independently halogen, —CN, C₁₋₆ alkyl, C₁₋₆ haloalkyl, C₁₋₆ alkoxy, or C₁₋₆haloalkoxy;
provided that when R¹¹ is H, R¹² is not —CH₃.

In some embodiments, the compound of formula (I) is a compound of Formula (Ia)

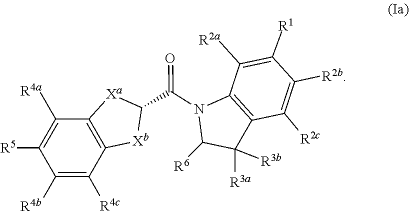

or a pharmaceutically acceptable salt thereof.

In some embodiments, the compound of formula (I) is a compound of Formula (II)

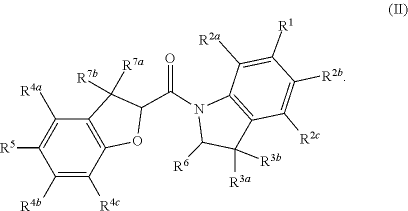

or a pharmaceutically acceptable salt thereof.

In some embodiments, the compound of formula (I) is a compound of Formula (IIa)

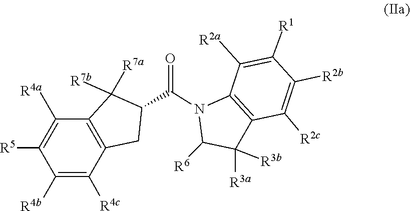

or a pharmaceutically acceptable salt thereof.

In some embodiments, the compound of formula (I) is a compound of Formula (III)

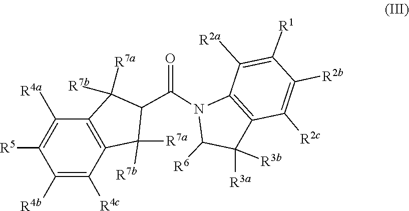

or a pharmaceutically acceptable salt thereof.

In some embodiments, the compound of formula (I) is a compound of Formula (IIIa)

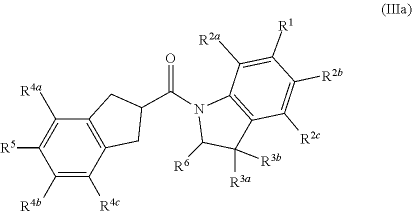

or a pharmaceutically acceptable salt thereof.

In some embodiments, the compound of formula (I) is a compound of Formula (IIIb)

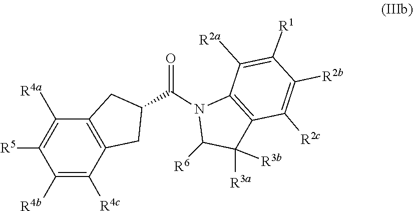

or a pharmaceutically acceptable salt thereof.

In some embodiments, the compound of formula (I) is a compound of Formula (IIIc)

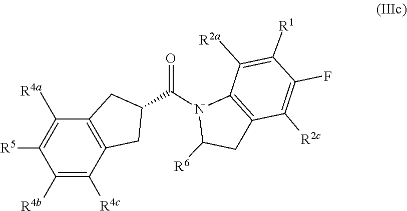

or a pharmaceutically acceptable salt thereof.

In some embodiments, the compound of formula (I) is a compound of Formula (IIId)

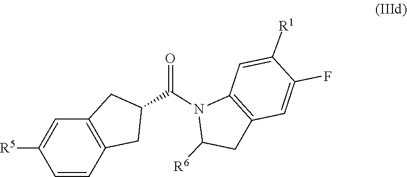

or a pharmaceutically acceptable salt thereof.

In some embodiments, the compound of formula (I) is a compound of Formula (IIIe)

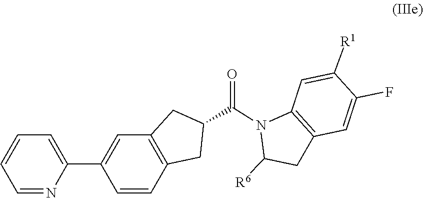

or a pharmaceutically acceptable salt thereof.

In some embodiments, the compound of Formula (I), (Ia), (II), (IIa), (III), (IIIa), (IIIb), or (IIIc), or a pharmaceutically acceptable salt thereof, is the compound wherein R^{2a} is H.

In some embodiments, the compound of Formula (I), (Ia), (II), (IIa), (III), (IIIa), (IIIb), or (IIIc), or a pharmaceutically acceptable salt thereof, is the compound wherein R^{2b} is halogen. In some embodiments, R^{2b} is F.

In some embodiments, the compound of Formula (I), (Ia), (II), (IIa), (III), (IIIa), (IIIb), or (IIIc), or a pharmaceutically acceptable salt thereof, is the compound wherein R^{2c} is H.

In some embodiments, the compound of Formula (I), (Ia), (II), (IIa), (III), (IIIa), or (IIIb), or a pharmaceutically acceptable salt thereof, is the compound wherein R^{3a} is H.

In some embodiments, the compound of Formula (I), (Ia), (II), (IIa), (III), (IIIa), or (IIIb), or a pharmaceutically acceptable salt thereof, is the compound wherein R^{3b} is H.

In some embodiments, the compound of Formula (I), (Ia), (II), (IIa), (III), (IIIa), (IIIb), or (IIIc), or a pharmaceutically acceptable salt thereof, is the compound wherein R^{4a} is H.

In some embodiments, the compound of Formula (I), (Ia), (II), (IIa), (III), (IIIa), (IIIb), or (IIIc), or a pharmaceutically acceptable salt thereof, is the compound wherein R^{4b} is H.

In some embodiments, the compound of Formula (I), (Ia), (II), (IIa), (III), (IIIa), (IIIb), or (IIIc), or a pharmaceutically acceptable salt thereof, is the compound wherein R^{4c} is H.

In some embodiments, the compound of Formula (I), (Ia), (II), (IIa), (III), (IIIa), (IIIb), (IIIc), or (IIId), or a pharmaceutically acceptable salt thereof, is the compound wherein R⁵ is heteroaryl; wherein the heteroaryl of R⁵ may be optionally substituted with one to three Z⁵, which may be the same or different.

In some embodiments, the compound of Formula (I), (Ia), (II), (IIa), (III), (IIIa), (IIIb), (IIIc), or (IIId), or a pharmaceutically acceptable salt thereof, is the compound wherein R⁵ is pyridyl optionally substituted with one to three Z⁵, which may be the same or different.

In some embodiments, the compound of Formula (I), (Ia), (II), (IIa), (III), (IIIa), (IIIb), (IIIc), or (IIId), or a pharmaceutically acceptable salt thereof, is the compound wherein R⁵ is pyridyl.

In some embodiments, the compound of Formula (I), (Ia), (II), (IIa), (III), (IIIa), (IIIb), (IIIc), (IIId), or (IIIe), or a pharmaceutically acceptable salt thereof, is the compound wherein R⁶ is H, C₁₋₆ alkyl or C₁₋₆ haloalkyl. In some embodiments, R⁶ is H. In some embodiments, R⁶ is —CH₃.

In some embodiments, the compound of Formula (I), (Ia), (II), (IIa), (III), (IIIa), (IIIb), (IIIc), (IIId), or (IIIe), or a pharmaceutically acceptable salt thereof, is the compound wherein R¹ is

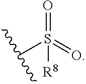

In some embodiments, the compound of Formula (I), (Ia), (II), (IIa), (III), (IIIa), (IIIb), (IIIc), (IIId), or (IIIe), or a pharmaceutically acceptable salt thereof, is the compound wherein
R¹ is

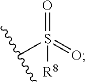

R⁸ is NHR⁹ or NR⁹R¹⁰;
each R⁹ and R¹⁰ is independently C₁₋₆ alkyl, C₁₋₆ haloalkyl, —COR^{9a}, or C₃₋₆ cycloalkyl; the C₁₋₆ alkyl of R⁹ or R¹⁰ is optionally substituted with —OR^{12a}; and each R^{9a} and R^{12a} is independently C₁₋₆ alkyl.

In some embodiments, the compound of Formula (I), (Ia), (II), (IIa), (III), (IIIa), (IIIb), (IIIc), (IIId), or (IIIe), or a pharmaceutically acceptable salt thereof, is the compound wherein
R¹ is

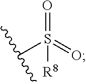

R⁸ is NHR⁹;
R⁹ is C₁₋₆ alkyl, or —COR^{9a}; and
R^{9a} is C₁₋₆ alkyl.

In some embodiments, the compound of Formula (I), (Ia), (II), (IIa), (III), (IIIa), (IIIb), (IIIc), (IIId), or (IIIe), or a pharmaceutically acceptable salt thereof, is the compound wherein R¹ is

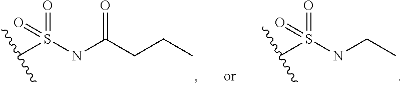

In some embodiments, the compound of Formula (I), (Ia), (II), (IIa), (III), (IIIa), (IIIb), (IIIc), (IIId), or (IIIe), or a pharmaceutically acceptable salt thereof, is the compound wherein R¹ is

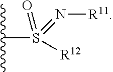

In some embodiments, the compound of Formula (I), (Ia), (II), (IIa), (III), (IIIa), (IIIb), (IIIc), (IIId), or (IIIe), or a pharmaceutically acceptable salt thereof, is the compound wherein
R¹ is

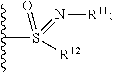

R¹¹ is H;
R¹² is —CH₂OR^{12a}, C₂₋₆ alkyl, C₁₋₆ haloalkyl, or C₃₋₆ cycloalkyl; the alkyl or cycloalkyl of
R¹² is optionally substituted with —OR^{12a}; and
R^{12a} is C₁₋₆ alkyl.

In some embodiments, the compound of Formula (I), (Ia), (II), (IIa), (III), (IIIa), (IIIb), (IIIc), (IIId), or (IIIe), or a pharmaceutically acceptable salt thereof, is the compound wherein
R¹ is

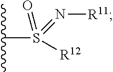

R¹¹ is H;
R¹² is C₂₋₆ alkyl or C₃₋₆ cycloalkyl; the alkyl or cycloalkyl or R¹² is optionally substituted with —OR^{12a}; and
R^{12a} is C₁₋₆ alkyl.

In some embodiments, the compound of Formula (I), (Ia), (II), (IIa), (III), (IIIa), (IIIb), (IIIc), (IIId), or (IIIe), or a pharmaceutically acceptable salt thereof, is the compound wherein R¹ is

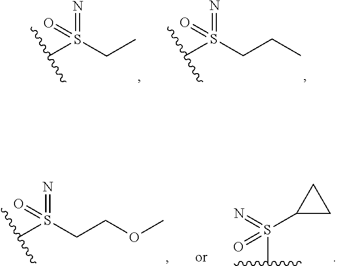

In some embodiments, the compound of Formula (I), (Ia), (II), (IIa), (III), (IIIa), (IIIb), (IIIc), (IIId), or (IIIe), or a pharmaceutically acceptable salt thereof, is the compound wherein
R¹ is

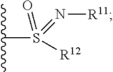

R¹¹ is C₁₋₆ alkyl;
R¹² is C₁₋₆ alkyl, C₁₋₆ haloalkyl, or C₃₋₆ cycloalkyl; the alkyl or cycloalkyl of R¹² is optionally substituted with —OR^{12a}; and
R^{12a} is C₁₋₆ alkyl.

In some embodiments, the compound of Formula (I), (Ia), (II), (IIa), (III), (IIIa), (IIIb), (IIIc), (IIId), or (IIIe), or a pharmaceutically acceptable salt thereof, is the compound wherein
R¹ is

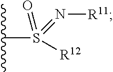

R¹¹ is C₁₋₆ alkyl;
R¹² is C₁₋₆ alkyl or C₃₋₆ cycloalkyl; the alkyl or cycloalkyl of R¹² is optionally substituted with —OR^{12a}; and
R^{12a} is C₁₋₆ alkyl.

In some embodiments, the compound of Formula (I), (Ia), (II), (IIa), (III), (IIIa), (IIIb), (IIIc), (IIId), or (IIIe), or a pharmaceutically acceptable salt thereof, is the compound wherein R¹ is

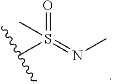 [SMILES: *S(C)(=O)=NC]

In some embodiments, the present disclosure provides a compound having a structure of a compound in Table 1, or pharmaceutically acceptable salt thereof. In some embodiments, the present disclosure provides a compound having a structure of a compound in Table 1, or a pharmaceutically acceptable salt, stereoisomer, or mixture of stereoisomers thereof.

TABLE 1
Some compounds

| Example # | structure |
|---|---|
| 1 | 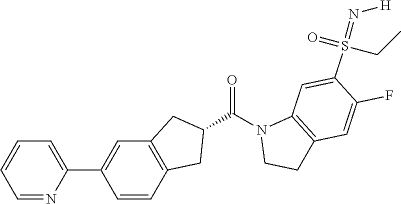 [SMILES: CCS(=N)(=O)c1cc2c(cc1F)CCN2C(=O)[C@@H]1Cc2ccc(-c3ccccn3)cc2C1] |
| 2 | 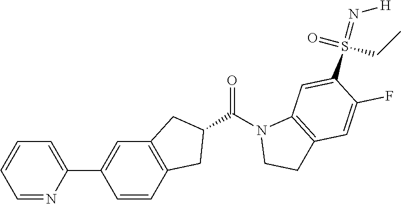 [SMILES: CC[S@@](=N)(=O)c1cc2c(cc1F)CCN2C(=O)[C@@H]1Cc2ccc(-c3ccccn3)cc2C1] |
| 3 | 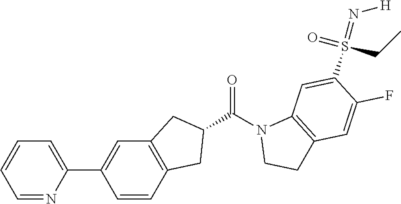 [SMILES: CC[S@](=N)(=O)c1cc2c(cc1F)CCN2C(=O)[C@@H]1Cc2ccc(-c3ccccn3)cc2C1] |
| 4 | 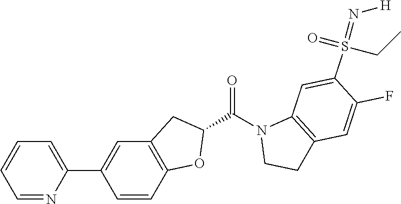 [SMILES: CCS(=N)(=O)c1cc2c(cc1F)CCN2C(=O)[C@H]1Cc2cc(-c3ccccn3)ccc2O1] |
| 5 | 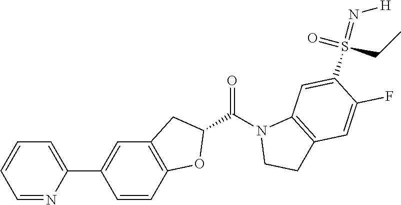 [SMILES: CC[S@](=N)(=O)c1cc2c(cc1F)CCN2C(=O)[C@H]1Cc2cc(-c3ccccn3)ccc2O1] |
| 6 | 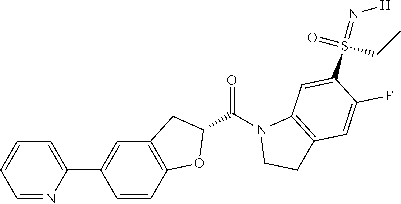 [SMILES: CC[S@@](=N)(=O)c1cc2c(cc1F)CCN2C(=O)[C@H]1Cc2cc(-c3ccccn3)ccc2O1] |
| 7 | 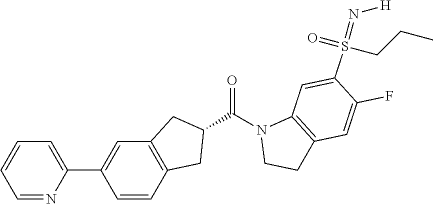 [SMILES: CCCS(=N)(=O)c1cc2c(cc1F)CCN2C(=O)[C@@H]1Cc2ccc(-c3ccccn3)cc2C1] |
| 8 | 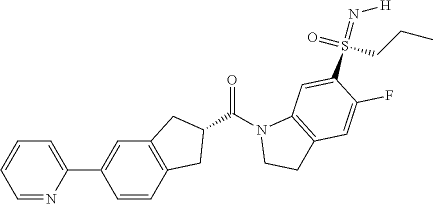 [SMILES: CCC[S@@](=N)(=O)c1cc2c(cc1F)CCN2C(=O)[C@@H]1Cc2ccc(-c3ccccn3)cc2C1] |
| 9 | 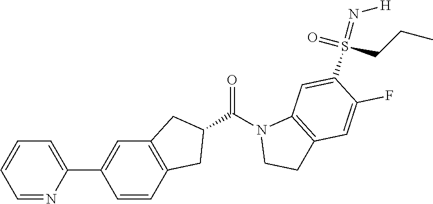 [SMILES: CCC[S@](=N)(=O)c1cc2c(cc1F)CCN2C(=O)[C@@H]1Cc2ccc(-c3ccccn3)cc2C1] |
| 10 | 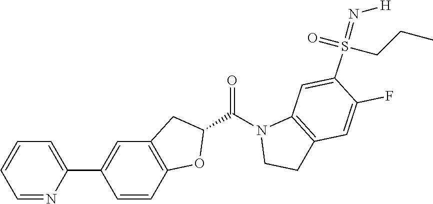 [SMILES: CCCS(=N)(=O)c1cc2c(cc1F)CCN2C(=O)[C@H]1Cc2cc(-c3ccccn3)ccc2O1] |
| 11 | 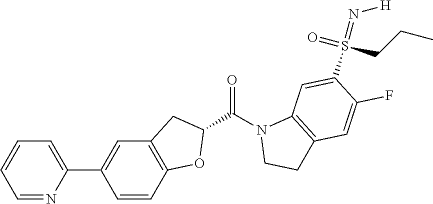 [SMILES: CCC[S@](=N)(=O)c1cc2c(cc1F)CCN2C(=O)[C@H]1Cc2cc(-c3ccccn3)ccc2O1] |
| 12 | 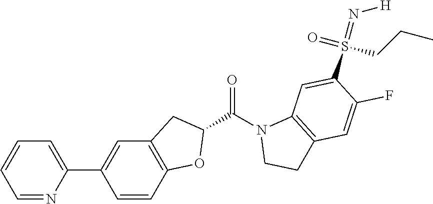 [SMILES: CCC[S@@](=N)(=O)c1cc2c(cc1F)CCN2C(=O)[C@H]1Cc2cc(-c3ccccn3)ccc2O1] |
| 13 | 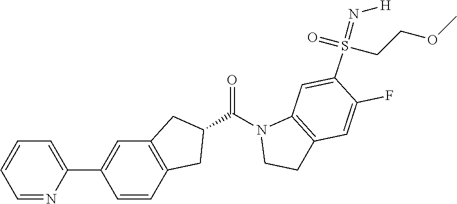 [SMILES: COCCS(=N)(=O)c1cc2c(cc1F)CCN2C(=O)[C@@H]1Cc2ccc(-c3ccccn3)cc2C1] |
| 14 | 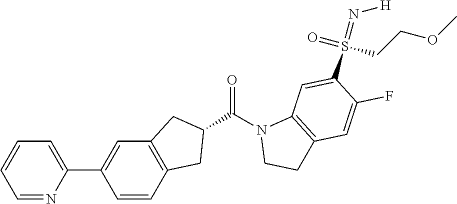 [SMILES: COCC[S@@](=N)(=O)c1cc2c(cc1F)CCN2C(=O)[C@@H]1Cc2ccc(-c3ccccn3)cc2C1] |
| 15 | 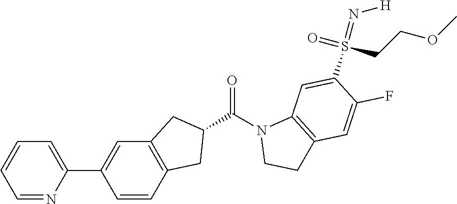 [SMILES: COCC[S@](=N)(=O)c1cc2c(cc1F)CCN2C(=O)[C@@H]1Cc2ccc(-c3ccccn3)cc2C1] |
| 16 | 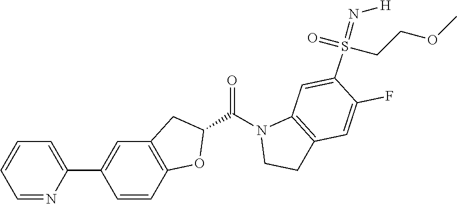 [SMILES: COCCS(=N)(=O)c1cc2c(cc1F)CCN2C(=O)[C@H]1Cc2cc(-c3ccccn3)ccc2O1] |
| 17 | 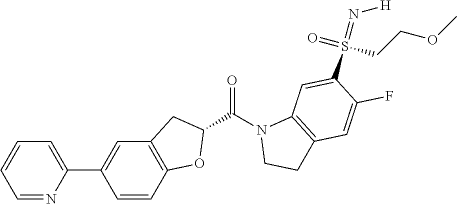 [SMILES: COCC[S@@](=N)(=O)c1cc2c(cc1F)CCN2C(=O)[C@H]1Cc2cc(-c3ccccn3)ccc2O1] |
| 18 | 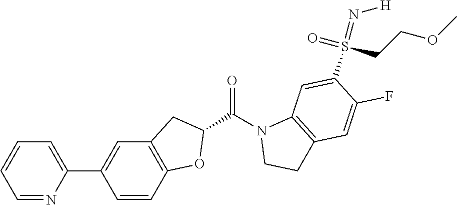 [SMILES: COCC[S@](=N)(=O)c1cc2c(cc1F)CCN2C(=O)[C@H]1Cc2cc(-c3ccccn3)ccc2O1] |
| 19 | 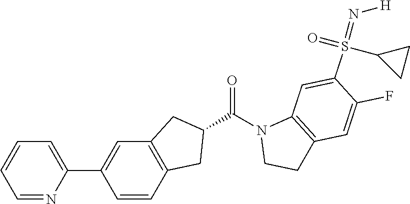 [SMILES: N=S(=O)(c1cc2c(cc1F)CCN2C(=O)[C@@H]1Cc2ccc(-c3ccccn3)cc2C1)C1CC1] |
| 20 | 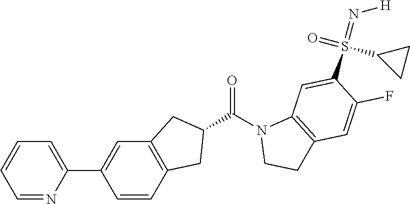 [SMILES: N=[S@](=O)(c1cc2c(cc1F)CCN2C(=O)[C@@H]1Cc2ccc(-c3ccccn3)cc2C1)C1CC1] |
| 21 | 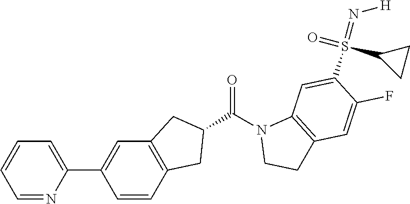 [SMILES: N=[S@@](=O)(c1cc2c(cc1F)CCN2C(=O)[C@@H]1Cc2ccc(-c3ccccn3)cc2C1)C1CC1] |
| 22 | 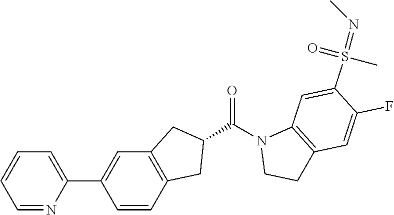 [SMILES: CN=S(C)(=O)c1cc2c(cc1F)CCN2C(=O)[C@@H]1Cc2ccc(-c3ccccn3)cc2C1] |
| 23 | 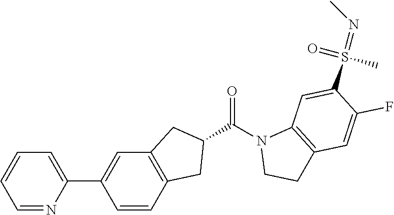 [SMILES: CN=[S@](C)(=O)c1cc2c(cc1F)CCN2C(=O)[C@@H]1Cc2ccc(-c3ccccn3)cc2C1] |
| 24 | 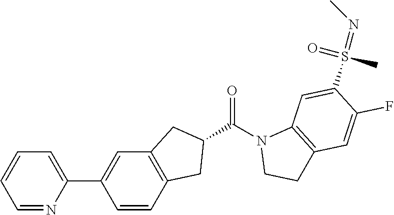 [SMILES: CN=[S@@](C)(=O)c1cc2c(cc1F)CCN2C(=O)[C@@H]1Cc2ccc(-c3ccccn3)cc2C1] |
| 25 | 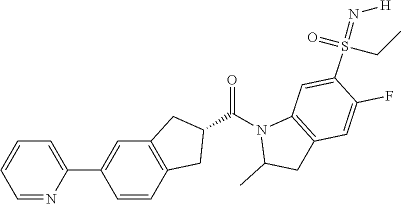 [SMILES: CCS(=N)(=O)c1cc2c(cc1F)CC(C)N2C(=O)[C@@H]1Cc2ccc(-c3ccccn3)cc2C1] |
| 26 | 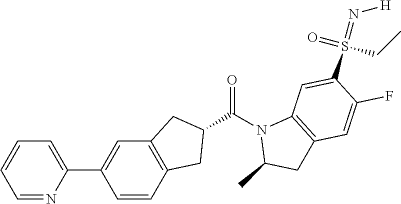 [SMILES: CC[S@@](=N)(=O)c1cc2c(cc1F)C[C@@H](C)N2C(=O)[C@@H]1Cc2ccc(-c3ccccn3)cc2C1] |
| 27 | 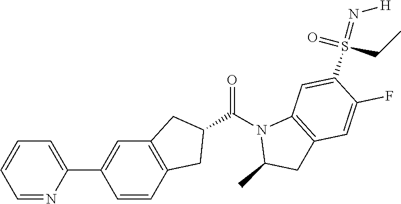 [SMILES: CC[S@](=N)(=O)c1cc2c(cc1F)C[C@@H](C)N2C(=O)[C@@H]1Cc2ccc(-c3ccccn3)cc2C1] |
| 28 | 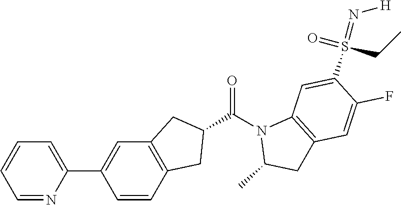 [SMILES: CC[S@](=N)(=O)c1cc2c(cc1F)C[C@H](C)N2C(=O)[C@@H]1Cc2ccc(-c3ccccn3)cc2C1] |
| 29 | 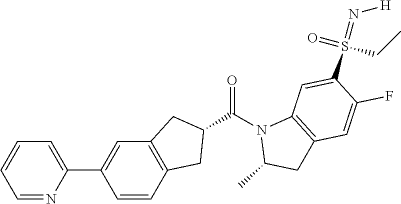 [SMILES: CC[S@@](=N)(=O)c1cc2c(cc1F)C[C@H](C)N2C(=O)[C@@H]1Cc2ccc(-c3ccccn3)cc2C1] |
| 30 | 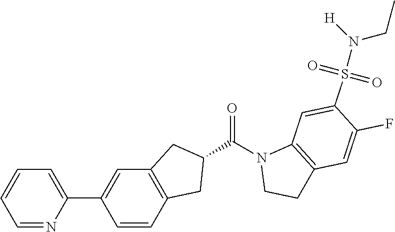 [SMILES: CCNS(=O)(=O)c1cc2c(cc1F)CCN2C(=O)[C@@H]1Cc2ccc(-c3ccccn3)cc2C1] |
| 31 | 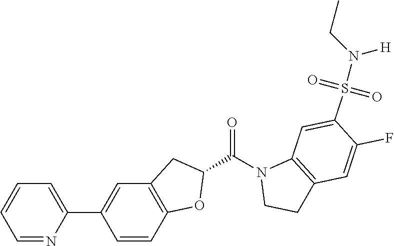 [SMILES: CCNS(=O)(=O)c1cc2c(cc1F)CCN2C(=O)[C@H]1Cc2cc(-c3ccccn3)ccc2O1] |
| 32 | 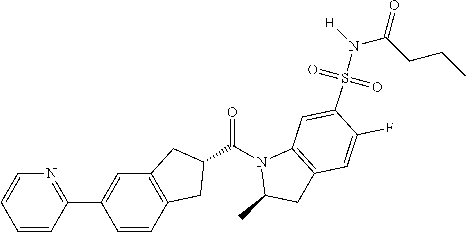 [SMILES: CCCC(=O)NS(=O)(=O)c1cc2c(cc1F)C[C@@H](C)N2C(=O)[C@@H]1Cc2ccc(-c3ccccn3)cc2C1] |

In some embodiments, the present disclosure provides a racemic mixture comprising the compound of Formula (I), (Ia), (II), (IIa), (III), (IIIa), (IIIb), (IIIc), (IIId), or (IIIe), or a pharmaceutically acceptable salt thereof. In some embodiments, the present disclosure provides a racemic mixture comprising the compound disclosed herein, or a pharmaceutically acceptable salt thereof. In some embodiments, the present disclosure provides a scalemic mixture comprising the compound of Formula (I), (Ia), (II), (IIa), (III), (IIIa), (IIIb), (IIIc), (IIId), or (IIIe), or a pharmaceutically acceptable salt thereof. In some embodiments, the present disclosure provides a scalemic mixture comprising the compound disclosed herein, or a pharmaceutically acceptable salt thereof.

One of skill in the art is aware that each and every embodiment of a group (e.g., R¹) disclosed herein may be combined with any other embodiment of each of the remaining groups (e.g., R^{2a}, R^{2b}, R^{2c}, R^{3a}, R^{3b}, etc.) to generate a complete compound of Formula (I), or any Formula described herein or a pharmaceutically acceptable salt, stereoisomer, mixture of stereoisomers, or tautomer thereof, each of which is deemed within the ambit of the present disclosure.

### Compositions and Kits

Compounds provided herein, or pharmaceutically acceptable salts thereof, are usually administered in the form of pharmaceutical compositions. Thus, provided herein are also pharmaceutical compositions that comprise one or more of the compounds provided herein or pharmaceutically acceptable salts, isomer, or a mixture thereof and one or more pharmaceutically acceptable vehicles selected from carriers, adjuvants and excipients. The compounds provided herein, or pharmaceutically acceptable salts thereof, may be the sole active ingredient or one of the active ingredients of the pharmaceutical compositions. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In some embodiments, provided herein are pharmaceutical compositions comprising a compound provided herein (i.e., a compound of Formula (I), (Ia), (II), (IIa), (III), (IIIa), (IIIb), (IIIc), (IIId), or (IIIe)), or a pharmaceutically acceptable salt thereof, and a pharmaceutically acceptable excipient or carrier. In some embodiments, the pharmaceutical compositions comprise a therapeutically effective amount of a compound provided herein, or a pharmaceutically acceptable salt thereof, and a pharmaceutically acceptable excipient or carrier.

In some embodiments, the pharmaceutical compositions provided herein further comprise one or more (i.e., one, two, three, four; one or two; one to three; or one to four) additional therapeutic agents, or a pharmaceutically acceptable salt thereof. In some embodiments, the pharmaceutical compositions further comprise a therapeutically effective amount of the one or more (i.e., one, two, three, four; one or two; one to three; or one to four) additional therapeutic agents, or a pharmaceutically acceptable salt thereof. In some embodiments, the one of more therapeutic agents are selected from Complement receptor 2 antagonists; Duffy antigen chemokine receptor modulators; Envelope glycoprotein GP350 modulators; Glucocorticoid receptor agonists; Helicase inhibitors; helicase-primase inhibitor; HIV gp160 protein inhibitors; HIV gp41 protein inhibitors; HIV-1 reverse transcriptase inhibitors; HLA class I antigen A-2 alpha modulators; HLA class I antigen A-24 alpha modulators; Human cytomegalovirus glycoprotein B modulators; Human cytomegalovirus glycoprotein H modulators; Human cytomegalovirus glycoprotein inhibitors; Human cytomegalovirus glycoprotein L modulators; Immunoglobulin G agonists; Interferon alpha 2 ligands; Interferon gamma receptor antagonists; Latent membrane protein 1 modulators; Latent membrane protein 2 modulators; Latent membrane protein 2 stimulators; Progesterone receptor agonists; Secreted protein BARF1 modulators; Serine threonine protein kinase UL97 modulators; T-cell surface glycoprotein CD8 stimulators; Thymidine kinase inhibitors; Trans acting transcription protein ICP4 modulators; Transferase inhibitors; Unspecified gene inhibitors; Adenosylhomocysteinase inhibitors; Basigin inhibitors; Basigin modulators; CCR5 chemokine modulators; CD4 agonists; CD4 modulators; CD89 agonists; CMV 65 kDa lower matrix phosphoprotein modulators; CRISPR associated endonuclease Cas9 modulators; Cyclin dependent kinase inhibitors; Cyclin dependent kinase inhibitors; Cyclin-dependent kinase-9 inhibitors; DNA polymerase inhibitors; DNA primase inhibitors; Endonuclease modulators; Epstein-Barr nuclear antigen 1 inhibitors; Epstein-Barr nuclear antigen 1 modulators; Epstein-Barr nuclear antigen 1 stimulators; Fatty acid synthase inhibitors; Herpesvirus envelope glycoprotein B stimulators; Herpesvirus envelope glycoprotein D inhibitors; Herpesvirus envelope glycoprotein D modulators; HIV gp120 protein inhibitors; HLA class I antigen A-11 alpha modulators; Hsp 90 inhibitors; Human cytomegalovirus glycoprotein B inhibitors; Human cytomegalovirus glycoprotein B modulators; Human cytomegalovirus glycoprotein inhibitors; Hyaluronidase inhibitors; Immunoglobulin agonists; Interferon alpha 1 ligands; Interferon alpha 2 ligands; Interferon alpha ligand inhibitors; Interferon alpha ligand modulators; Interferon beta ligands; Large terminase protein inhibitors; LAT gene inhibitors; NAD-dependent deacetylase sirtuin modulators; Nicotinic acetylcholine receptor antagonists; NKG2D ligand modulators; Nucleotidyltransferase inhibitors; Protein Jumonji inhibitors; Ribonuclease stimulators; Serine threonine protein kinase UL97 inhibitors; Syntaxin-5 inhibitors; TAT protein modulators; T-cell surface glycoprotein CD8 stimulators; TLR-4 agonists; and viral ribonucleotide reductase inhibitors. In some embodiments, the one of more therapeutic agents are selected from famciclovir, acyclovir, and valacyclovir.

In some embodiments, provided herein are pharmaceutical compositions comprising a therapeutically effective amount of a compound provided herein, or a pharmaceutically acceptable salt thereof, and a pharmaceutically acceptable excipient.

The pharmaceutical compositions may be administered in either single or multiple doses. The pharmaceutical compositions may be administered by various methods including, for example, rectal, buccal, intranasal and transdermal routes. In some embodiments, the pharmaceutical compositions may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. In some embodiments, the compounds, or pharmaceutically acceptable salts thereof, and pharmaceutical compositions disclosed herein are administered by subcutaneous injection.

The pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

In some embodiments, the sterile injectable preparation disclosed herein may also be a sterile injectable solution or suspension prepared from a reconstituted lyophilized powder in a non-toxic parenterally acceptable diluent or solvent, such as a solution in 1,3-butane-diol. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. In certain embodiments the suspension is a microsuspension. In certain embodiments the suspension is a nanosuspension.

In some embodiments, formulations suitable for parenteral administration (e.g., intramuscular (IM) and subcutaneous (SC) administration) will include one or more excipients. Excipients should be compatible with the other ingredients of the formulation and physiologically innocuous to the recipient thereof. Examples of suitable excipients are well known to the person skilled in the art of parenteral formulation and may be found e.g., in Handbook of Pharmaceutical Excipients (eds. Rowe, Sheskey & Quinn), 6th edition 2009. Examples of solubilizing excipients in a parenteral formulation (e.g., an SC or IM formulation) include, but are not limited to, polysorbates (such as polysorbate 20 or 80) and poloxamers (such as poloxamer 338, 188, or 207). In some embodiments, the compounds, or pharmaceutically acceptable salts thereof, and pharmaceutical compositions disclosed herein are administered with implants.

Oral administration may be another route for administration of the compounds provided herein or pharmaceutically acceptable salts thereof. Administration may be via, for example, capsule or enteric coated tablets. In making the pharmaceutical compositions that include at least one compound provided herein or pharmaceutically acceptable salts, isomer, or a mixture thereof, the active ingredient (such as a compound provided herein) is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the pharmaceutical compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose or any combinations thereof. The pharmaceutical compositions can additionally include lubricating agents such as talc, magnesium stearate, and mineral oil; wetting agents; emulsifying and suspending agents; preserving agents such as methyl and propylhydroxy-benzoates; sweetening agents; and flavoring agents; or any combinations thereof.

The pharmaceutical compositions that include at least one compound described herein or pharmaceutically acceptable salts, isomer, or a mixture thereof can be formulated so as to provide quick, sustained or delayed release of the active ingredient (such as a compound provided herein) after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present disclosure employs transdermal delivery devices (“patches”). Such transdermal patches may be used to provide continuous or discontinuous infusion of the compounds provided herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or pharmaceutically acceptable salts, isomer, or a mixture thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds provided herein or pharmaceutically acceptable salts thereof may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with materials such as shellac, cetyl alcohol, and cellulose acetate.

Pharmaceutical compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In one embodiment, provided herein are kits that comprise a compound provided herein, (i.e., a compound of Formula (I), (Ia), (II), (IIa), (III), (IIIa), (IIIb), (IIIc), (IIId), or (IIIe)), or a pharmaceutically acceptable salt, stereoisomer, prodrug, or solvate thereof, and suitable packaging. In some embodiments, the kit further comprises instructions for use. In some embodiments, the kit comprises a compound provided herein (i.e., a compound of Formula (I), (Ia), (II), (IIa), (III), (IIIa), (IIIb), (IIIc), (IIId), or (IIIe)), or a pharmaceutically acceptable salt, stereoisomer, prodrug, or solvate thereof, and a label and/or instructions for use of the compounds in the treatment of the indications, including the diseases or conditions, described herein.

In some embodiments, the kits further comprise one or more (i.e., one, two, three, four; one or two; one to three; or one to four) additional therapeutic agents, or a pharmaceutically acceptable salt thereof.

In one embodiment, provided herein are articles of manufacture that comprise a compound described herein or pharmaceutically acceptable salts, isomer, or a mixture thereof in a suitable container. In some embodiments, the container may be a vial, jar, ampoule, preloaded syringe, or intravenous bag.

### Method

The methods provided herein may be applied to cell populations in vivo or ex vivo. “In vivo” means within a living individual, as within an animal or human. In this context, the methods provided herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. Exemplary tissue samples include tumors and biopsies thereof. In this context, the present disclosure may be used for a variety of purposes, including therapeutic and experimental purposes. For example, the present disclosure may be used ex vivo to determine the optimal schedule and/or dosing of administration of a compound as disclosed herein for a given cell type, individual, and other parameters. Information gleaned from such use may be used for experimental purposes or in the clinic to set protocols for in vivo treatment. Other ex vivo uses for which the present disclosure may be suited are described below or will become apparent to those skilled in the art. The selected compounds may be further characterized to examine the safety or tolerance dosage in human or non-human subjects. Such properties may be examined using commonly known methods to those skilled in the art.

In one embodiment, the present disclosure provides a method of treating or preventing a herpesvirus infection in a patient in need thereof comprising administering to the patient a therapeutically effective amount of a compound provided herein, or a pharmaceutically acceptable salt thereof, or a pharmaceutical composition provided herein.

In some embodiments, the methods provided herein further comprise administering a therapeutically effective amount of one, two, three, or four additional therapeutic agents, or a pharmaceutically acceptable salt thereof.

The disclosure further relates to the use of compounds disclosed herein for the treatment and/or prophylaxis of diseases and/or conditions through inhibiting the helicase primase of herpesviruses by said compounds. Further, the present disclosure relates to the use of said compounds for the preparation of a medicament for the treatment and/or prophylaxis of a herpesvirus associated disease and/or condition through inhibiting helicase primase of herpesviruses by said compounds. In some embodiments the herpesvirus associated disease or condition is alleviated by inhibiting herpesvirus helicase primase. In some embodiments, the present disclosure relates to the use of the compounds disclosed herein for the preparation of a medicament for the treatment and/or prophylaxis of HSV-1 or HSV-2 associated disease and/or condition through inhibiting the helicase primase by said compounds.

Medicaments as referred to herein can be prepared by conventional processes, including the combination of a compound according to the present disclosure and a pharmaceutically acceptable carrier.

In some embodiments, provided herein is a method of the inhibiting the helicase primase of herpesviruses comprising administering to a patient in need thereof (e.g., a patient having a herpesvirus associated disease or condition) a therapeutically effective amount of a compound of Formula (I), (Ia), (II), (IIa), (III), (IIIa), (IIIb), (IIIc), (IIId), or (IIIe), or a pharmaceutically acceptable salt thereof, or a composition comprising a compound of Formula (I), (Ia), (II), (IIa), (III), (IIIa), (IIIb), (IIIc), (IIId), or (IIIe), or a pharmaceutically acceptable salt thereof.

In some embodiments, provided herein is a method of inhibiting the helicase primase of HSV-1 or HSV-2 comprising administering to a patient in need thereof (e.g., a patient having a HSV-1 or HSV-2 associated disease or condition) a therapeutically effective amount of a compound of Formula (I), (Ia), (II), (IIa), (III), (IIIa), (IIIb), (IIIc), (IIId), or (IIIe), or a pharmaceutically acceptable salt thereof, or a composition comprising a compound of Formula (I), (Ia), (II), (IIa), (III), (IIIa), (IIIb), (IIIc), (IIId), or (IIIe), or a pharmaceutically acceptable salt thereof. In some embodiments, provided herein is a method of inhibiting the helicase primase of HSV-1 or HSV-2 comprising administering to a patient in need thereof (e.g., a patient having a HSV-1 or HSV-2 associated disease or condition) a therapeutically effective amount of a compound disclosed herein, or a pharmaceutically acceptable salt thereof, or a composition comprising a compound disclosed herein, or a pharmaceutically acceptable salt thereof.

In some embodiments, provided herein is a method of reducing the proliferation of a virus comprising contacting the virus with a compound of Formula (I), (Ia), (II), (IIa), (III), (IIIa), (IIIb), (IIIc), (IIId), or (IIIe), or a pharmaceutically acceptable salt thereof, and inhibiting helicase primase in the virus. In some embodiments, provided herein is a method of reducing the proliferation of herpesviruses comprising contacting the virus with a compound of Formula (I), (Ia), (II), (IIa), (III), (IIIa), (IIIb), (IIIc), (IIId), or (IIIe), or a pharmaceutically acceptable salt thereof, and inhibiting helicase primase in the virus. In some embodiments, provided herein is a method of reducing the proliferation of HSV-1 or HSV-2 comprising contacting the virus with a compound disclosed herein, or a pharmaceutically acceptable salt thereof, and inhibiting helicase primase in the virus.

In some embodiments, provided herein is a method of treating a disorder induced, exacerbated, or accelerated by herpesviruses, comprising administering to a patient a therapeutically effective amount of a compound of Formula (I), (Ia), (II), (IIa), (III), (IIIa), (IIIb), (IIIc), (IIId), or (IIIe), or a pharmaceutically acceptable salt thereof, or a composition comprising a compound of Formula (I), (Ia), (II), (IIa), (III), (IIIa), (IIIb), (IIIc), (IIId), or (IIIe), or a pharmaceutically acceptable salt thereof, to a patient in need thereof. In some embodiments, provided herein is a method of treating a disorder induced, exacerbated, or accelerated by HSV-1 or HSV-2, comprising administering to a patient a therapeutically effective amount of a compound of Formula (I), (Ia), (II), (IIa), (III), (IIIa), (IIIb), (IIIc), (IIId), or (IIIe), or a pharmaceutically acceptable salt thereof, or a composition comprising a compound of Formula (I), (Ia), (II), (IIa), (III), (IIIa), (IIIb), (IIIc), (IIId), or (IIIe), or a pharmaceutically acceptable salt thereof, to a patient in need thereof. In some embodiments, the disorder is genital herpes, herpes labialis, HSV keratitis, HSV encephalitis, or disseminated HSV. In some embodiments, the disorder is genital herpes.

In some embodiments, provided herein is the use of a compound of Formula (I), (Ia), (II), (IIa), (III), (IIIa), (IIIb), (IIIc), (IIId), or (IIIe), or a pharmaceutically acceptable salt thereof, in the treatment of a viral infection. In some embodiments, provided herein is the use of a compound of Formula (I), (Ia), (II), (IIa), (III), (IIIa), (IIIb), (IIIc), (IIId), or (IIIe), or a pharmaceutically acceptable salt thereof, in the treatment of a viral infection caused by herpesviruses. In some embodiments, provided herein is the use of a compound of Formula (I), (Ia), (II), (IIa), (III), (IIIa), (IIIb), (IIIc), (IIId), or (IIIe), or a pharmaceutically acceptable salt thereof, in the treatment of a viral infection caused by HSV-1 or HSV-2.

In some embodiments, provided herein is the use of a compound of Formula (I), (Ia), (II), (IIa), (III), (IIIa), (IIIb), (IIIc), (IIId), or (IIIe), or a pharmaceutically acceptable salt thereof, for the preparation of a medicament for preventing/treating a viral infection. In some embodiments, provided herein is the use of a compound of Formula (I), (Ia), (II), (IIa), (III), (IIIa), (IIIb), (IIIc), (IIId), or (IIIe), or a pharmaceutically acceptable salt thereof, for the preparation of a medicament for preventing/treating a viral infection caused by herpesviruses. In some embodiments, provided herein is the use of a compound of Formula (I), (Ia), (II), (IIa), (III), (IIIa), (IIIb), (IIIc), (IIId), or (IIIe), or a pharmaceutically acceptable salt thereof, for the preparation of a medicament for preventing/treating a viral infection caused by HSV-1 or HSV-2.

In some embodiments, provided herein is the compound of Formula (I), (Ia), (II), (IIa), (III), (IIIa), (IIIb), (IIIc), (IIId), or (IIIe), or pharmaceutically acceptable salt thereof, for use in therapy. In some embodiments, provided herein is the compound of Formula (I), (Ia), (II), (IIa), (III), (IIIa), (IIIb), (IIIc), (IIId), or (IIIe), or pharmaceutically acceptable salt thereof, for use in a method of treating a viral infection caused by HSV-1 or HSV-2.

### Administration

The compounds of the present disclosure or pharmaceutically acceptable salts thereof (also referred to herein as the active ingredients) can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the preferred route may vary with, for example, the condition of the recipient. An advantage of certain compounds disclosed herein, or pharmaceutically acceptable salts thereof, is that they are orally bioavailable and can be dosed orally.

A compound of the present disclosure, or a pharmaceutically acceptable salt thereof, may be administered to an individual in accordance with an effective dosing regimen for a desired period of time or duration, such as at least about one day, at least about 2 days, at least about 3 days, at least about 4 days, at least about 5 days, at least about 6 days, at least about one week, at least about 2 weeks, at least about 3 weeks, at least about one month, at least about 2 months, at least about 3 months, at least about 6 months, or at least about 12 months or longer. In some embodiments, the compound, or a pharmaceutically acceptable salt thereof, is administered on a daily or intermittent schedule for the duration of the individual's life.

The specific dose level of a compound of the present disclosure, or a pharmaceutically acceptable salt thereof, for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound provided herein, or a pharmaceutically acceptable salt thereof, per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The dosage may also be described as a total amount of a compound described herein, or a pharmaceutically acceptable salt thereof, administered per dose. The dosage or dosing frequency of a compound of the present disclosure, or a pharmaceutically acceptable salt thereof, may be adjusted over the course of the treatment, based on the judgment of the administering physician.

The compounds of the present disclosure, or pharmaceutically acceptable salts thereof, may be administered to an individual (e.g., a human) in a therapeutically effective amount. In some embodiments, the compound of Formula (I), (Ia), (II), (IIa), (III), (IIIa), (IIIb), (IIIc), (IIId), or (IIIe), or a pharmaceutically acceptable salt thereof, is administered once daily, once weekly, once monthly, once every two months, once every three months, or once every six months. In some embodiments, the compound of Formula (I), (Ia), (II), (IIa), (III), (IIIa), (IIIb), (IIIc), (IIId), or (IIIe), or a pharmaceutically acceptable salt thereof, is administered once daily. In some embodiments, the compound of Formula (I), (Ia), (II), (IIa), (III), (IIIa), (IIIb), (IIIc), (IIId), or (IIIe), or a pharmaceutically acceptable salt thereof, is administered once weekly. In some embodiments, the compound of Formula (I), (Ia), (II), (IIa), (III), (IIIa), (IIIb), (IIIc), (IIId), or (IIIe), or a pharmaceutically acceptable salt thereof, is administered once monthly. In some embodiments, the compound of Formula (I), (Ia), (II), (IIa), (III), (IIIa), (IIIb), (IIIc), (IIId), or (IIIe), or a pharmaceutically acceptable salt thereof, is administered once every two months. In some embodiments, the compound of Formula ((I), (Ia), (II), (IIa), (III), (IIIa), (IIIb), (IIIc), (IIId), or (IIIe), or a pharmaceutically acceptable salt thereof, is administered once every three months. In some embodiments, the compound of Formula (I), (Ia), (II), (IIa), (III), (IIIa), (IIIb), (IIIc), (IIId), or (IIIe), or a pharmaceutically acceptable salt thereof, is administered once every six months.

The compounds provided herein, or pharmaceutically acceptable salts thereof, can be administered by any useful route and means, such as by oral or parenteral (e.g., intravenous) administration. Therapeutically effective amounts of the compound, or a pharmaceutically acceptable salt thereof, may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the compounds provided herein, or pharmaceutically acceptable salts thereof, include from about 30 μg to about 300 μg per day, from about 0.3 mg to about 30 mg per day, or from about 30 mg to about 300 mg per day, or from about 300 mg to about 1000 mg per day.

A compound of the present disclosure, or a pharmaceutically acceptable salt thereof, may be combined with one or more additional therapeutic agents in any dosage amount of the compound of the present disclosure or a pharmaceutically acceptable salt thereof (e.g., from 1 mg to 1000 mg of compound). Therapeutically effective amounts may include from about 0.1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose, or such as from about 0.01 mg per dose to about 1000 mg per dose, or such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose, or such as from about 1 mg per dose to about 1000 mg per dose. Other therapeutically effective amounts of the compound of Formula (I), (IIa), (IIb), (IIc), (IId), (IIe), or (IIf), or a pharmaceutically acceptable salt thereof, are about 50, 100, 125, 150, 175, 200, 225, 250, 275, or 300 mg per dose. Other therapeutically effective amounts of the compound of Formula (I), (Ia), (II), (IIa), (III), (IIIa), (IIIb), (IIIc), (IIId), or (IIIe), or pharmaceutically acceptable salts thereof, are about 300, 325, 350, 375, 400, 425, 450, 475, 500, 525, 550, 575, 600, 625, 650, 675, 700, 725, 750, 775, 800, 825, 850, 875, 900, 925, 950, 975, or about 1000 mg per dose.

In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 1000 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 900 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 800 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 700 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 600 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 500 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 400 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 300 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 200 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 100 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 75 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 50 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 25 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 20 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 15 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 10 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1 mg to about 5 mg.

In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 50 mg, about 75 mg, about 100 mg, about 125 mg, about 150 mg, about 175 mg, about 200 mg, about 225 mg, about 275 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, or about 1050 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 5 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 100 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 150 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 200 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 250 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 300 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 350 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 400 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 450 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 500 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 550 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 600 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 650 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 700 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 750 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 800 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 850 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 900 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 950 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1000 mg. In some embodiments, a therapeutically effective amount of the compound of Formula (I), (Ia), (II), (IIa), (III), (IIIa), (IIIb), (IIIc), (IIId), or (IIIe), or a pharmaceutically acceptable salt thereof, is about 1050 mg. When administered orally, the total weekly dosage for a human subject may be between about 1 mg and 1,000 mg/week, between about 10-500 mg/week, between about 50-300 mg/week, between about 75-200 mg/week, or between about 100-150 mg/week. In some embodiments, the total weekly dosage for a human subject may be about 100, 200, 300, 400, 500, 600, 700, 800, 900, or 1000 mg/week administered in a single dose. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 100 mg administered in a single dose. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 150 mg administered in a single dose. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 200 mg administered in a single dose. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 250 mg administered in a single dose. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 300 mg administered in a single dose. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 350 mg administered in a single dose. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 400 mg administered in a single dose. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 450 mg administered in a single dose. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 500 mg administered in a single dose. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 600 mg administered in a single dose. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 700 mg administered in a single dose. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 800 mg administered in a single dose. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 900 mg administered in a single dose. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 1000 mg administered in a single dose.

When administered orally, the total monthly dosage for a human subject of a compound of Formula (I), (Ia), (II), (IIa), (III), (IIIa), (IIIb), (IIIc), (IIId), or (IIIe), or a pharmaceutically acceptable salt thereof, may be between about 500 mg and 1,000 mg/month, between about 600-900 mg/month, or between about 700-800 mg/month. In some embodiments, the total weekly dosage for a human subject of a compound of Formula (I), (Ia), (II), (IIa), (III), (IIIa), (IIIb), (IIIc), (IIId), or (IIIe), or a pharmaceutically acceptable salt thereof, may be about 100, 200, 300, 400, 500, 600, 700, 800, 900, or 1000 mg/week administered in a single dose. In some embodiments, the total monthly dosage for a human subject of a compound of Formula (I), (Ia), (II), (IIa), (III), (IIIa), (IIIb), (IIIc), (IIId), or (IIIe), or a pharmaceutically acceptable salt thereof, may be about 500 mg administered in a single dose. In some embodiments, the total monthly dosage for a human subject may be about 550 mg administered in a single dose. In some embodiments, the total monthly dosage for a human subject of a compound of Formula (I), (Ia), (II), (IIa), (III), (IIIa), (IIIb), (IIIc), (IIId), or (IIIe), or a pharmaceutically acceptable salt thereof, may be about 600 mg administered in a single dose. In some embodiments, the total monthly dosage for a human subject of a compound of Formula (I), (Ia), (II), (IIa), (III), (IIIa), (IIIb), (IIIc), (IIId), or (IIIe), or a pharmaceutically acceptable salt thereof, may be about 650 mg administered in a single dose. In some embodiments, the total monthly dosage for a human subject of a compound of Formula (I), (Ia), (II), (IIa), (III), (IIIa), (IIIb), (IIIc), (IIId), or (IIIe), or a pharmaceutically acceptable salt thereof, may be about 700 mg administered in a single dose. In some embodiments, the total monthly dosage for a human subject of a compound of Formula (I), (Ia), (II), (IIa), (III), (IIIa), (IIIb), (IIIc), (IIId), or (IIIe), or a pharmaceutically acceptable salt thereof, may be about 750 mg administered in a single dose. In some embodiments, the total monthly dosage for a human subject of a compound of Formula (I), (Ia), (II), (IIa), (III), (IIIa), (IIIb), (IIIc), (IIId), or (IIIe), or a pharmaceutically acceptable salt thereof, may be about 800 mg administered in a single dose. In some embodiments, the total monthly dosage for a human subject of a compound of Formula (I), (Ia), (II), (IIa), (III), (IIIa), (IIIb), (IIIc), (IIId), or (IIIe), or a pharmaceutically acceptable salt thereof, may be about 850 mg administered in a single dose. In some embodiments, the total monthly dosage for a human subject of a compound of Formula (I), (Ia), (II), (IIa), (III), (IIIa), (IIIb), (IIIc), (IIId), or (IIIe), or a pharmaceutically acceptable salt thereof, may be about 900 mg administered in a single dose. In some embodiments, the total monthly dosage for a human subject of a compound of Formula (I), (Ia), (II), (IIa), (III), (IIIa), (IIIb), (IIIc), (IIId), or (IIIe), or a pharmaceutically acceptable salt thereof, may be about 950 mg administered in a single dose. In some embodiments, the total monthly dosage for a human subject of a compound of Formula (I), (Ia), (II), (IIa), (III), (IIIa), (IIIb), (IIIc), (IIId), or (IIIe), or a pharmaceutically acceptable salt thereof, may be about 1000 mg administered in a single dose.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a compound provided herein, or a pharmaceutically acceptable salt thereof, is administered once daily in a method disclosed herein. In some embodiments, a compound provided herein, or a pharmaceutically acceptable salt thereof, is administered twice daily in a method disclosed herein.

In some embodiments, a compound provided herein, or a pharmaceutically acceptable salt thereof, is administered once daily in a method disclosed herein. In some embodiments, a compound provided herein, or a pharmaceutically acceptable salt thereof, is administered once weekly in a method disclosed herein. In some embodiments, a compound provided herein, or a pharmaceutically acceptable salt thereof, is administered once monthly in a method disclosed herein. In some embodiments, a compound provided herein, or a pharmaceutically acceptable salt thereof, is administered once every two months in a method disclosed herein. In some embodiments, a compound provided herein, or a pharmaceutically acceptable salt thereof, is administered once every three months in a method disclosed herein. In some embodiments, a compound provided herein, or a pharmaceutically acceptable salt thereof, is administered once every six months in a method disclosed herein.

In some embodiments, a compound provided herein, or a pharmaceutically acceptable salt thereof, is administered orally in a single dose of about 100 mg once weekly. In some embodiments, a compound provided herein, or a pharmaceutically acceptable salt thereof, is administered orally in a single dose of about 150 mg once weekly. In some embodiments, a compound provided herein, or a pharmaceutically acceptable salt thereof, is administered orally in a single dose of about 200 mg once weekly. In some embodiments, a compound provided herein, or a pharmaceutically acceptable salt thereof, is administered orally in a single dose of about 250 mg once weekly. In some embodiments, a compound provided herein, or a pharmaceutically acceptable salt thereof, is administered orally in a single dose of about 300 mg once weekly. In some embodiments, a compound provided herein, or a pharmaceutically acceptable salt thereof, is administered orally in a single dose of about 350 mg once weekly. In some embodiments, a compound provided herein, or a pharmaceutically acceptable salt thereof, is administered orally in a single dose of about 400 mg once weekly. In some embodiments, a compound provided herein, or a pharmaceutically acceptable salt thereof, is administered orally in a single dose of about 450 mg once weekly. In some embodiments, a compound provided herein, or a pharmaceutically acceptable salt thereof, is administered orally in a single dose of about 500 mg once weekly. In some embodiments, a compound provided herein, or a pharmaceutically acceptable salt thereof, is administered orally in a single dose of about 600 mg once weekly.

In some embodiments, a compound provided herein, or a pharmaceutically acceptable salt thereof, is administered orally in a single dose of about 500 mg once monthly. In some embodiments, a compound provided herein, or a pharmaceutically acceptable salt thereof, is administered orally in a single dose of about 550 mg once monthly. In some embodiments, a compound provided herein, or a pharmaceutically acceptable salt thereof, is administered orally in a single dose of about 600 mg once monthly. In some embodiments, a compound provided herein, or a pharmaceutically acceptable salt thereof, is administered orally in a single dose of about 650 mg once monthly. In some embodiments, a compound provided herein, or a pharmaceutically acceptable salt thereof, is administered orally in a single dose of about 700 mg once monthly. In some embodiments, a compound provided herein, or a pharmaceutically acceptable salt thereof, is administered orally in a single dose of about 750 mg once monthly. In some embodiments, a compound provided herein, or a pharmaceutically acceptable salt thereof, is administered orally in a single dose of about 800 mg once monthly. In some embodiments, a compound provided herein, or a pharmaceutically acceptable salt thereof, is administered orally in a single dose of about 850 mg once monthly. In some embodiments, a compound provided herein, or a pharmaceutically acceptable salt thereof, is administered orally in a single dose of about 900 mg once monthly. In some embodiments, a compound provided herein, or a pharmaceutically acceptable salt thereof, is administered orally in a single dose of about 950 mg once monthly. In some embodiments, a compound provided herein, or a pharmaceutically acceptable salt thereof, is administered orally in a single dose of about 1000 mg once monthly.

The frequency of dosage of the compound of the present disclosure, or a pharmaceutically acceptable salt thereof, will be determined by the needs of the individual patient and can be, for example, once per day, once per week, once per month, once per every two months, once per every three months, or once per every six months. Administration of the compound, or a pharmaceutically acceptable salt thereof, continues for as long as necessary to treat the herpesvirus infection, including an HSV-1 and an HSV-2 infection, or any other indication described herein. For example, a compound, or a pharmaceutically acceptable salt thereof, can be administered to a human suffering from a herpesvirus infection, including an HSV-1 and an HSV-2 infection, for the duration of the human's life.

Administration can be intermittent, with a period of several or more days during which a patient receives a daily dose of the compound of the present disclosure, or a pharmaceutically acceptable salt thereof, followed by a period of several or more days during which a patient does not receive a daily dose of the compound or a pharmaceutically acceptable salt thereof. For example, a patient can receive a dose of the compound, or a pharmaceutically acceptable salt thereof, every other day, or three times per week. Again by way of example, a patient can receive a dose of the compound, or a pharmaceutically acceptable salt thereof, each day for a period of from 1 to 14 days, followed by a period of 7 to 21 days during which the patient does not receive a dose of the compound, or a pharmaceutically acceptable salt thereof, followed by a subsequent period (e.g., from 1 to 14 days) during which the patient again receives a daily dose of the compound, or a pharmaceutically acceptable salt thereof. Alternating periods of administration of the compound, or a pharmaceutically acceptable salt thereof, followed by non-administration of the compound, or a pharmaceutically acceptable salt thereof, can be repeated as clinically required to treat the patient.

The compounds of the present disclosure, or pharmaceutically acceptable salts thereof, or the pharmaceutical compositions of the present disclosure may be administered once, twice, three, or four times daily, using any suitable mode described above. Also, administration or treatment with the compounds, or pharmaceutically acceptable salts thereof, may be continued for a number of days; for example, commonly treatment would continue for at least 7 days, 14 days, or 28 days, for one cycle of treatment. Treatment cycles are well known for herpesvirus infections, including an HSV-1 and an HSV-2 infection. In some embodiments, treatment cycles are frequently alternated with resting periods of about 1 to 28 days, commonly about 7 days or about 14 days, between cycles. The treatment cycles, in other embodiments, may also be continuous.

The effective dosage of active ingredient employed may vary depending on the particular compound employed, the mode of administration, the condition being treated and the severity of the condition being treated. Such dosage may be ascertained readily by a person skilled in the art.

### Combinations

In some embodiments, a compound of Formula (I), (Ia), (II), (IIa), (III), (IIIa), (IIIb), (IIIc), (IIId), or (IIIe), provided herein, or pharmaceutically acceptable salt thereof, is administered in combination with one or more additional therapeutic agents to treat or prevent a disease or condition disclosed herein. In some embodiments, the one or more additional therapeutic agents are one, two, three, or four additional therapeutic agents. In some embodiments, the one or more additional therapeutic agents are one additional therapeutic agent.

In some embodiments, the one or more additional therapeutic agents are two additional therapeutic agents. In some embodiments, the one or more additional therapeutic agents are three additional therapeutic agents. In some embodiments, the one or more additional therapeutic agents are four additional therapeutic agents.

In some embodiments, the pharmaceutical compositions provided herein have a compound of Formula (I), (Ia), (II), (IIa), (III), (IIIa), (IIIb), (IIIc), (IIId), or (IIIe) provided herein, or pharmaceutically acceptable salt thereof, and one or more additional therapeutic agents. In some embodiments, the one or more additional therapeutic agents are one, two, three, or four additional therapeutic agents. In some embodiments, the one or more additional therapeutic agents are one additional therapeutic agent. In some embodiments, the one or more additional therapeutic agents are two additional therapeutic agents. In some embodiments, the one or more additional therapeutic agents are three additional therapeutic agents. In some embodiments, the one or more additional therapeutic agents are four additional therapeutic agents.

In some embodiments, the one or more additional therapeutic agents include, e.g., DNA polymerase inhibitors such as, brincidofovir; Protein Jumonji inhibitors such as, ML-324, dimethyloxaloylglycine; Interferon alpha 2 ligand modulators such as, interferon alfa-2b, Alpharekin®, recombinant human interferon alfa-2b follow-on biologic, Herpferon®, Anterferon®; Nicotinic acetylcholine receptor antagonists such as, RPI-MN; Virus specific T cell therapies such as off-the-shelf single-virus specific T-cell (VST) therapy, ALVR-108, multi-virus-specific T cells TI1; Other drugs for the treatment of Herpesvirus infection such as, BOR-15001L7, lidocaine, Bryostatin-23; or any combination thereof.

In some embodiments, the one or more additional therapeutic agents include, e.g., anti-Herpesvirus envelope glycoprotein D antibodies such as m27f; Progesterone receptor agonists such as, levonorgestrel; or HIV-1 reverse transcriptase inhibitors such as, tenofovir; or any combination thereof.

In some embodiments, the one or more additional therapeutic agents include, e.g., Endonuclease modulators such as, meganucleases; Interferon alpha ligand/Interferon gamma receptor antagonists such as, Anaferon®, or any combination thereof.

In some embodiments, the one or more additional therapeutic agents include, e.g., CCR5 chemokine modulators; DNA polymerase inhibitors; DNA primase inhibitors; Fatty acid synthase inhibitors; Glucocorticoid receptor agonists; Helicase inhibitors; Herpesvirus envelope glycoprotein D inhibitors; Hsp 90 inhibitors; Human cytomegalovirus glycoprotein inhibitors; Hyaluronidase inhibitors; Interferon alpha 1 ligands; Interferon alpha 2 ligands; Interferon beta ligands; T-cell surface glycoprotein CD8 stimulators; or Cyclin dependent kinase inhibitors, or any combination thereof.

In some embodiments, the one or more additional therapeutic agents include, e.g., DNA primase-helicase inhibitors such as, amenamevir, pritelivir, IM-250; DNA polymerase inhibitors such as famciclovir, penciclovir, valaciclovir, acyclovir, foscarnet sodium; CCR5 chemokine modulators/Human cytomegalovirus glycoprotein inhibitors such as, MB-66; Hsp 90 inhibitors such as, BJ-B11; Glucocorticoid receptor agonists such as, hydrocortisone; Interferon alpha 1 ligand modulators such as, interferon alpha 1b, recombinant human interferon alpha 1b; Interferon alpha 2 ligand modulators such as, recombinant human interferon alpha-2b, KW-045; Interferon beta ligand modulators such as, interferon beta-1a (RebiSmart™); anti-HSV envelope glycoprotein D antibodies, such as UB-621; T-cell surface glycoprotein CD8 stimulators such as, immunogenic peptides, Fatty acid synthase inhibitors such as, TVB-2640; anti-herpes simplex virus monoclonal antibodies such as, HDIT-101; or other drugs for the treatment of Herpes simplex virus infection such as, idoxuridine, BTL-TML-HSV, docosanol, MAR-8644, MAR-8658, NV-HHV-101, PRL-01, HN-0037; or any combination thereof.

In some embodiments, the one or more additional therapeutic agents include, e.g., Hyaluronidase inhibitors such as, astodrimer (SPL-7013); Live-attenuated HSV vaccines such as, RVx-101 HSV-1; Live-attenuated HSV vaccine with deletions in UL20 and UL53 such as, VC2; Live-attenuated HSV vaccine mutated in R2 coding region of UL37 such as, R2; or HSV-2 subunit trivalent vaccine (containing gC2, gD2, gE2) such as, HSV-2 Trivalent Vaccine; or any combination thereof.

In some embodiments, the one or more additional therapeutic agents include, e.g., live attenuated recombinant vaccines such as, AuroVax; HSV-2 replication-defective vaccine with UL5 and UL29 deleted such as, HSV-529; mRNA vaccine targeted against HSV-2 disease such as, mRNA-1608; or any combination thereof.

In some embodiments, the one or more additional therapeutic agents include, e.g., CD4 agonists; CD89 agonists; Duffy antigen chemokine receptor modulators; Herpesvirus envelope glycoprotein D inhibitors; HIV gp120 protein inhibitors; HIV gp160 protein inhibitors; HIV gp41 protein inhibitors; Immunoglobulin G agonists; Nicotinic acetylcholine receptor antagonists; TAT protein modulators; T-cell surface glycoprotein CD8 stimulators; or TLR-4 agonist; or any combination thereof.

In some embodiments, the one or more additional therapeutic agents include, e.g., Nicotinic acetylcholine receptor antagonists such as, RPI-78M; anti-herpes simplex virus monoclonal antibodies such as, HDIT-101; anti-herpesvirus envelope glycoprotein D antibodies such as, UB-621; TLR-4 agonists such as, IDC-G103 vaccines; Inactivated HSV-1 and HSV-2 vaccines such as, Vitaherpavac®; live Inactivated HSV-1 and HSV-2 vaccines such as, Theravax-HSV-2 vaccine; formalin-inactivated herpesvirus (FI—HSV2) vaccine; or RBT-26 T-cell-based subunit vaccine; DNA vaccines such as, pDNA/rVSV vector vaccine; or other drugs for the treatment of HSV-2 such as, EBT-105, Alloferon™; or any combination thereof.

In some embodiments, the one or more additional therapeutic agents include, e.g., HIV gp120/gp160/gp41 protein inhibitors such as, griffithsin; Hyaluronidase inhibitors such as, astodrimer (SPL-7013); CD4 agonist/T-cell surface glycoprotein CD8 vaccine such as GENO-2; TAT protein modulators such as HerpesVaxTat® vaccines; CD89 agonist/Duffy antigen chemokine receptor modulator/Immunoglobulin G agonist such as, glycoprotein D+liposome encapsulated glycoprotein D boost vaccine; or vaccines such as, Profavax-HSV-2 vaccine; HSV-2 mRNA vaccine; glycoprotein D DNA vaccine; or any combination thereof.

In some embodiments, the one or more additional therapeutic agents include, e.g., DNA vaccines such as, HSV-2 vaccine Admedus; RNA vaccines such as, GSK-4108771A; Live attenuated virus vaccines such as, EXD-12; live-attenuated delta-gD2 based viral vaccines; Vaccines such as, NE-gD2 intranasal nanoemulsion NE-based adjuvanted HSV-2 vaccine; or other drugs for the treatment of HSV-2 such as, EBT-105, Alloferon™; or any combination thereof.

In some embodiments, the one or more additional therapeutic agents include, e.g., antimicrobial peptoids; CD4 modulators; CRISPR associated endonuclease Cas9 modulators; Cyclin-dependent kinase-9 inhibitors; DNA polymerase inhibitors; Nicotinic acetylcholine receptor antagonists; TAT protein modulators; Thymidine kinase inhibitors; or viral envelope protein inhibitors; or any combination thereof.

In some embodiments, the one or more additional therapeutic agents include, e.g., DNA polymerase inhibitors/Thymidine kinase inhibitors such as acyclovir; CRISPR associated endonuclease Cas9 modulators such as, EBT-104; Nicotinic acetylcholine receptor antagonists such as, RPI-78M; Cyclin-dependent kinase-9 inhibitors such as, FIT-039; Other drugs for HSV-1 treatment such as, ZEP-3Na, MXB-009; CD4 modulator peptide vaccine such as, CEL-1000; Vaccines such as, Inactivated HSV-1 and HSV-2 vaccines such as, Vitaherpavac® (anti-herpes vaccine); live Inactivated HSV-1 and HSV-2 vaccines such as, Theravax-HSV-1 vaccine; or Viral envelope protein inhibitors such as, MXB-005; or a combination thereof.

In some embodiments, the one or more additional therapeutic agents include, e.g., TAT protein modulators such as HerpesVaxTat® vaccine; or Vaccines such as, Profavax-HSV-1 vaccine; or a combination thereof.

In some embodiments, the one or more additional therapeutic agents include, e.g., Live attenuated virus vaccines such as, EXD-12; or live-attenuated delta-gD2 based viral vaccines; or a combination thereof.

In some embodiments, the one or more additional therapeutic agents include, e.g., DNA polymerase inhibitors; Herpesvirus envelope glycoprotein D inhibitors; or Interferon alpha 2 ligand; or a combination thereof.

In some embodiments, the one or more additional therapeutic agents include, e.g., DNA polymerase inhibitors such as, famciclovir, penciclovir; Interferon alpha 2 ligand modulators such as, Yallaferon®; anti-Herpesvirus envelope glycoprotein D antibodies such as, UB-621; or other drugs for treatment of genital herpes such as, interferon gamma, Alloferon™, ZEP-3Na; or a combination thereof.

In some embodiments, the one or more additional therapeutic agents include, e.g., Vaccines such as, NE-gD2 intranasal nanoemulsion NE-based adjuvanted vaccine; or other drugs for the treatment and prevention of genital herpes such as, SQX-77, anti-STI antibodies; or a combination thereof.

In some embodiments, the one or more additional therapeutic agents include, e.g., SAPNs (self-assembling protein nanoparticles) based herpes vaccine.

In some embodiments, the one or more additional therapeutic agents include, e.g., CMV 65 kDa lower matrix phosphoprotein modulators; DNA polymerase inhibitors; G protein coupled receptor homolog US28 antagonists; Herpesvirus envelope glycoprotein B stimulators; HLA class I antigen A-11 alpha modulators; HLA class I antigen A-2 alpha modulators; HLA class I antigen A-24 alpha modulators; Human cytomegalovirus glycoprotein B inhibitors; Human cytomegalovirus glycoprotein B modulators; Human cytomegalovirus glycoprotein H modulators; Human cytomegalovirus glycoprotein inhibitors; Large terminase protein inhibitors; Ribonuclease stimulators; Serine threonine protein kinase UL97 modulators; Syntaxin-5 inhibitors; Transferase inhibitors; or Viral ribonucleotide reductase inhibitors; or a combination thereof.

In some embodiments, the one or more additional therapeutic agents include, e.g., DNA polymerase inhibitors such as, ganciclovir, fomivirsen, fomivirsen sodium, valganciclovir; DNA polymerase inhibitor/Serine threonine protein kinase UL97 modulators such as, filociclovir; G protein coupled receptor homolog US28 antagonists such as, SYN-002; Large terminase protein inhibitors such as, AIC-387, AIC-476; Serine threonine protein kinase UL97 inhibitors such as, maribavir; Viral ribonucleotide reductase inhibitors such as, didox; Ribonuclease stimulators such as, ranpirnase; HLA class I antigen A-11 alpha modulators/HLA class I antigen A-2 alpha modulators/HLA class I antigen A-24 alpha modulators such as, allogenic anti-CMV-TCR-T-cell therapy, YT-CMV-22, YT-CMV-27 and YT-CMV-45; Human cytomegalovirus glycoprotein B inhibitors such as, CMV-345; Other drugs for treatment of CMV such as, USC-505, USC-596, CMV pp65 and ppM83 derived peptides, artemifone (BAY-44-9585), PG-36, CMX-16669, HN-0141, ALVR-105, NPP-669, CMV pH4 human immunoglobulin, Cytovir™ anti-viral cytotoxic T-cell therapy, CMV TCR-transduced T-cells, adimlecleucel; or Polyclonal antibodies such as, Cytogam®; or a combination thereof.

In some embodiments, the one or more additional therapeutic agents include, e.g., Syntaxin-5 inhibitors such as, Retro-94; Large terminase protein inhibitors such as, letermovir; DNA polymerase inhibitors such as, valganciclovir; anti-CMV antibodies such as, BT-084 (Cytotect® CP); Human cytomegalovirus glycoprotein B and glycoprotein H modulator vaccines such as mRNA-1647; CMV 65 kDa lower matrix phosphoprotein vaccines such as, IRB-12022; or Vaccines such as, mRNA-based vaccine; BD-03 plasmid DNA vaccine; V-212 heat-treated varicella zoster virus vaccine; protein subunit vaccines such as, VBI-1501A; CMV-MVA pentamer vaccine (RhUL128C-MVA); CMV-MVA Triplex vaccine; VLP based vaccine SPYVLP-102; or any combination thereof.

In some embodiments, the one or more additional therapeutic agents include, e.g., Human cytomegalovirus glycoprotein inhibitors such as, CMV-IVIG; Additional drugs for the treatment or prevention of CMV such as, artemisinin derivatives, NPC-21; Herpesvirus envelope glycoprotein B stimulator vaccine such as, HB-101; CMV 65 kDa lower matrix phosphoprotein modulator vaccine such as, AVX-601; Vaccines such as, CMV vaccine; CMVpp65 peptide vaccine; V-160; or Multivirus-specific cytotoxic T-cell therapy; or any combination thereof.

In some embodiments, the one or more additional therapeutic agents include, e.g., DNA polymerase inhibitors such as, ganciclovir, fomivirsen, cidofovir, valganciclovir, foscarnet sodium.

In some embodiments, the one or more additional therapeutic agents include, e.g., DNA polymerase inhibitors; Helicase inhibitors; Immunoglobulin agonists; Interferon alpha 2 ligands; Interferon alpha ligand modulators; Interferon beta ligands; or TLR-4 agonists; or any combination thereof.

In some embodiments, the one or more additional therapeutic agents include, e.g., DNA polymerase inhibitors such as, penciclovir, famciclovir, valaciclovir, acyclovir, USC-373; DNA primase inhibitor-helicase inhibitor such as, amenamevir; Interferon alpha 2 ligand modulators such as, interferon alfa-2b, Yallaferon®, pegylated interferon alpha-1b, INTEFEN®, interferon alpha-2a, Anterferon®; Interferon beta ligand modulators such as, RebiSmart™; Immunoglobulin agonists such as, Varicellon®, Zoster Immunoglobulin-VF; Vaccines such as, varicella vaccine (live attenuated); MMRV vaccine; herpes zoster vaccine; zoster recombinant adjuvanted vaccine; or Drugs for treatment of Varicella zoster virus infection such as, HerpeCide™; or any combination thereof.

In some embodiments, the one or more additional therapeutic agents include, e.g., Vaccines such as, ProQuad®; Priorix-Tetra® (MeMuRu-OKA); protein subunit TLR-4 agonist vaccines such as, CRV-101; VZV ORF29 mutant-based vaccine; varicella vaccine (live attenuated); chickenpox vaccine Sinovac; Varilrix® vaccine (VZV-OKA-strain); attenuated recombinant subunit vaccine containing gE such as, GSK-137173A; protein subunit vaccines such as, SP-0204; pneumococcal conjugate vaccines such as, SP-0202; triple live-attenuated vaccines such as, M-M-RvaxPRO®; adenovirus-vectored vaccine such as, VTP-400; recombinant varicella-zoster virus vaccine; recombinant herpes zoster vaccine; inactivated varicella-zoster vaccine; Live attenuated viral vaccines such as, NBP-608, Suduvax® II, VZV-7D; or anti-Varicella Zoster virus antibodies such as, VariZIG®; or any combination thereof.

In some embodiments, the one or more additional therapeutic agents include, e.g., drugs for treatment or prevention of Varicella zoster virus infection such as, OV-02; or vaccines such as, EG-HZ; or any combination thereof.

In some embodiments, the one or more additional therapeutic agents include, e.g., interferon alpha 2 ligand; DNA polymerase inhibitors; transferase inhibitors; CRISPR associated endonuclease Cas9 modulators; LAT gene inhibitor; or gene inhibitors; or any combination thereof.

In some embodiments, the one or more additional therapeutic agents include, e.g., DNA polymerase inhibitors such as, acyclovir, ganciclovir; Interferon alpha 2 ligand modulators such as, interferon alfa-2b, Anterferon®; CRISPR associated endonuclease Cas9 modulator/gene inhibitors such as, HSV-1-targeted CRISPR/Cas9 gene therapy; LAT gene inhibitors such as, IFNγ/LAT siRNA gene therapy (rdHSV1 vector, herpes simplex keratitis); additional drugs for the treatment of herpetic keratitis such as, EKC-Cide™; or any combination thereof.

In some embodiments, the one or more additional therapeutic agents include, e.g., Basigin inhibitors; Envelope glycoprotein GP350 modulators; Epstein-Barr nuclear antigen 1 inhibitors; Epstein-Barr nuclear antigen 1 stimulators; HLA class I antigen A-11 alpha modulators; HLA class I antigen A-2 alpha modulators; HLA class I antigen A-24 alpha modulators; Human cytomegalovirus glycoprotein B modulators; Human cytomegalovirus glycoprotein H modulators; Human cytomegalovirus glycoprotein L modulators; Latent membrane protein 1 modulators; Latent membrane protein 2 stimulators; NKG2D ligand modulators; or Secreted protein BARF1 modulators; or any combination thereof.

In some embodiments, the one or more additional therapeutic agents include, e.g., NKG2D ligand modulators such as, pamidronic acid; Epstein-Barr nuclear antigen 1 inhibitors such as, VK-2019, anti-Epstein-Barr virus (EBV) peptides; HLA class I antigen A-II alpha/HLA class I antigen A-2 alpha/HLA class I antigen A-24 alpha modulators such as, allogenic anti-EBV-TCR-T-cells, YT-EBV-44; Additional drugs for EBV treatment such as, ALVR-105, anti-viral cytotoxic T-cell therapy; Epstein-Barr nuclear antigen/Latent membrane protein 1 and protein 2/Secreted protein BARF1 modulators such as, baltaleucel-T (CMD-003); or mRNA vaccines such as, mRNA-1195; or any combination thereof.

In some embodiments, the one or more additional therapeutic agents include, e.g., basigin modulators such as, EBV gH/gL/gp42 vaccine; or basigin modulator/Envelope glycoprotein GP350 modulator/Human cytomegalovirus glycoprotein B modulator/Human cytomegalovirus glycoprotein H modulator/Human cytomegalovirus glycoprotein L modulator such as, mRNA-1189 vaccine; vaccines such as, EBV gH/gL vaccine; P-989; mRNA vaccine; or EBV cancer vaccine (mRNA, LPP nanoparticle); or any combination thereof.

In some embodiments, the one or more additional therapeutic agents include, e.g., Epstein-Barr nuclear antigen 1/Latent membrane protein 2 stimulators such as MVA-based vaccines; Multivirus-specific cytotoxic T-cell therapy; or vaccines such as, EBV-VLP vaccine; or any combination thereof.

In some embodiments, the one or more additional therapeutic agents include, e.g., DNA polymerase inhibitors.

In some embodiments, the one or more additional therapeutic agents include, e.g., DNA polymerase inhibitors such as, brincidofovir; Drugs for herpesvirus type 6 treatment such as, ALVR-105; Multivirus-specific cytotoxic T-cell therapy; or HHV-6B glycoprotein complex gH/gL/gQ1/gQ2 subunit vaccine; or any combination thereof.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that these examples are exemplary and not exhaustive. Many changes can be made in the specific embodiments which are disclosed and still obtain a like or similar result without departing from the spirit and scope of the disclosure.

Compounds disclosed herein can be prepared according to the procedures of the following Schemes and Examples, using appropriate materials and are further exemplified by the following specific examples. Moreover, by utilizing the procedures described herein, in conjunction with ordinary skills in the art, additional compounds of the present disclosure claimed herein can be readily prepared. The examples further illustrate details for the preparation of the compounds of the present disclosure. Those skilled in the art will readily understand that known variations of the conditions and processes of the following preparative procedures can be used to prepare these compounds. For synthesizing compounds which are embodiments described in the present disclosure, inspection of the structure of the compound to be synthesized will provide the identity of each substituent group. In some cases, the identity of the final product can render apparent the identity of the necessary starting materials by a process of inspection, given the examples herein. Compounds can be isolated in the form of their pharmaceutically acceptable salts, such as those described above. Compounds described herein are typically stable and isolatable at room temperature and pressure.

An illustration of the preparation of compounds disclosed herein is shown below. Unless otherwise indicated, variables have the same meaning as described above. The examples presented below are intended to illustrate particular embodiments of the disclosure. Suitable starting materials, building blocks and reagents employed in the synthesis as described below are commercially available from AbovChem, Acros Organics, Astatech, Combi Blocks, Oakwood Chemical, or Sigma-Aldrich, for example, or can be routinely prepared by procedures described in the literature, for example in “March's Advanced Organic Chemistry: Reactions, Mechanisms, and Structure”, 5^{th} Edition; John Wiley & Sons or T. Eicher, S. Hauptmann “The Chemistry of Heterocycles; Structures, Reactions, Synthesis and Application”, 2^{nd} edition, Wiley-VCH 2003; Fieser et al. “Fiesers' Reagents for organic Synthesis” John Wiley & Sons 2000.

List of Abbreviations and Acronyms

| Abbreviation/Acronym | Meaning |
|---|---|
| Ac | Acetate |
| AcOH | Acetic acid |
| aq. | Aqueous |
| Ar | Argon |
| Bn | Benzyl |
| BnSH | Benzyl mercaptan |
| Boc | tert-Butyloxycarbonyl |
| Boc₂O | Di-tert-butyl dicarbonate |
| Bu₄N | Tetrabutylammonium |
| DCM | Dichloromethane |
| DIEA or DIPEA | N,N-diisopropylethylamine |
| DMAP | 4-dimethylaminopyridine |
| DMF | N,N-Dimethylformamide |
| DMSO | Dimethylsulfoxide |
| eq. or equiv | equivalent |
| Et | Ethyl |
| Et₃N | Triethylamine |
| EtOAc | Ethyl acetate |
| h or hr(s) | Hour(s) |
| HPLC | High pressure liquid chromatography |
| i-Pr | Isopropyl |
| LCMS | Liquid chromatography mass spectrometry |
| Me | Methyl |
| MeOH | Methanol |
| MPLC | Medium pressure liquid chromatography |
| MS | Mass spectrometry |
| m/z | Mass to charge ratio |
| NMR | Nuclear magnetic resonance spectroscopy |
| PhMe | Toluene |
| RT or rt | Room temperature |
| sat. or satd. | Saturated |
| SFC | supercritical fluid chromatography |
| t-Bu or tBu | tert-Butyl |
| TCFH | chloro(dimethylamino)-N,N- |
| | dimethylmethaniminium hexafluorophosphate |
| TFA | Trifluoroacetic acid |
| THF | Tetrahydrofuran |

### General Procedure A

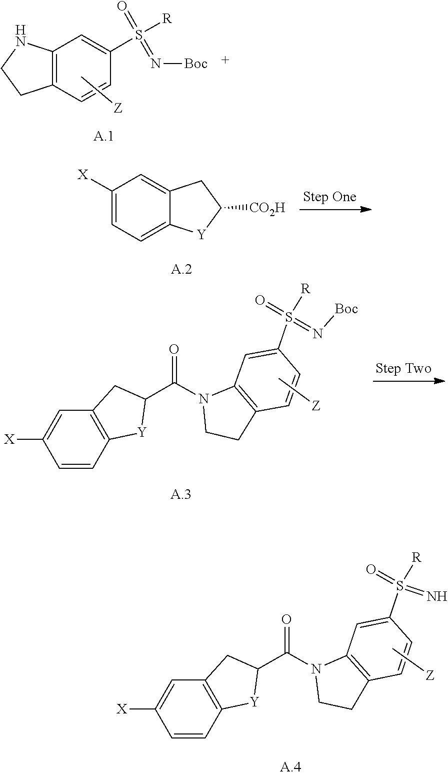

Step One (amide coupling): To a solution of indoline of type A.1 (1 equiv), carboxylic acid of type A.2 (1 equiv), and chloro-N,N,N′,N′-tetramethylformamidinium hexafluorophosphate (1.1 equiv) in MeCN (0.05 M) was added N-methylimidazole (2.2 equiv) at 0° C. The reaction was warmed to RT for 2 hr. The reaction mixture was diluted with water and extracted with ethyl acetate. The combined organic fractions were washed with brine, dried over Na₂SO₄, filtered and concentrated under reduced pressure to give a residue. The residue was redissolved in DCM and silica gel was added. The resultant mixture was concentrated under reduced pressure to give a flowing solid, which was purified by flash chromatography on silica gel (0-50% EtOAc in hexanes) to afford the coupling product of type A.3.

Step Two (deprotection): The amide of type A.3 obtained in Step One was dissolved in DCM (0.05 M). To this solution at RT was added trifluoroacetic acid (8 equiv) and the reaction was allowed to stir for 2 hours. The reaction mixture was diluted with water and extracted with DCM. The combined organic fractions were washed with brine, dried over Na₂SO₄, filtered and concentrated under reduced pressure. The residue was purified using reverse phase HPLC to afford the desired product of type A.4.

### Preparation of Intermediate 1:

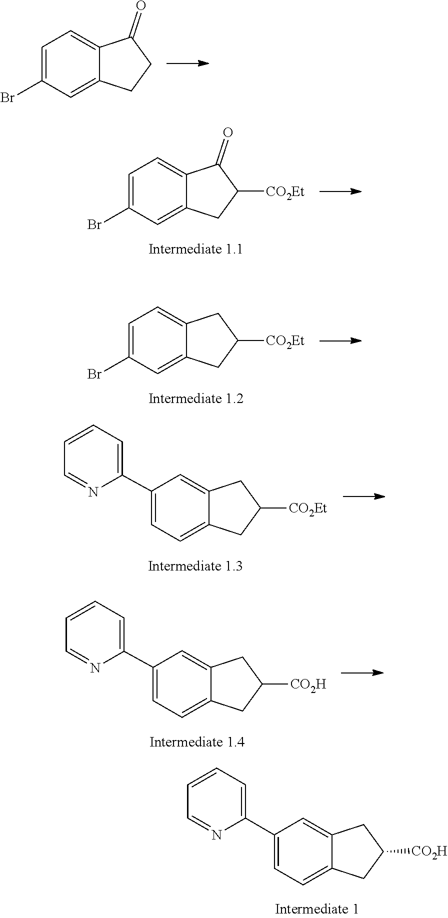 [SMILES: O=C1CCc2cc(Br)ccc21 ; CCOC(=O)C1Cc2cc(Br)ccc2C1=O ; CCOC(=O)C1Cc2ccc(Br)cc2C1 ; CCOC(=O)C1Cc2ccc(-c3ccccn3)cc2C1 ; O=C(O)C1Cc2ccc(-c3ccccn3)cc2C1 ; O=C(O)[C@@H]1Cc2ccc(-c3ccccn3)cc2C1]

Preparation of Intermediate 1.1: To a stirred suspension of 60% NaH in mineral oil (22.72 g, 2 equiv) in THF (1000 mL) at 0° C. was added diethyl carbonate (86 mL, 1.5 equiv). To this was added 5-bromo-indan-1-one (100 g, 473.1 mmol) portion wise at this temperature. The reaction mixture continued to stir at this temperature until gas evolution subsided. The reaction mixture was slowly heated to 50° C. and stirring maintained for 2 h. Progress of the reaction was monitored by TLC, and after consumption of the starting material, the reaction mixture was allowed to cool to RT. The reaction was diluted with EtOAc (10V). To this was added 3N aq HCl dropwise. Both layers were separated. Aqueous layer was extracted with EtOAc (2×5V). The combined organic layer was washed with brine solution (5V), dried (Na₂SO₄) and evaporated to afford crude product. The crude product was purified by column chromatography on silica gel to afford Intermediate 1.1. LCMS: 283.0 [M+H]⁺.

Preparation of Intermediate 1.2: To a solution of Intermediate 1.1 (56 g, 197.9 mmol) in TFA (280 mL) at 0° C. was added triethyl silane (224 mL) dropwise and the reaction mixture was stirred at rt for 24 h. Progress of the reaction was monitored by TLC. After consumption of the starting materials, the reaction mixture was evaporated to dryness under reduced pressure to afford crude product. The crude product was purified by column chromatography on silica gel to afford Intermediate 2.2. LC/MS: 269.0 [M+H]⁺.

Preparation of Intermediate 1.3: A stirred solution of Intermediate 1.2 (40 g, 148.7 mmol) and 2-tributylstannylpyridine (57.5 g, 1.05 equiv) in 1,4 dioxane (400 mL) was degassed for 10 min using argon at which time Pd(PPh₃)₄ (8.6 g, 5 mol %) was added and again degassed for another 10 min. The reaction mixture was heated to 90° C. for 16 h. The reaction mixture was filtered through a Celite pad and the Celite pad was washed with ethyl acetate twice. The combined filtrate was evaporated to dryness to afford crude product. The crude product was purified by column chromatography on silica gel to afford Intermediate 1.3. LC/MS: 268.1 [M+H].

Preparation of Intermediate 1.4: To a stirred solution of Intermediate 1.3 (35 g, 131.1 mmol) in methanol (350 mL) at 0° C. was added 2N aq NaOH solution (140 mL, 2.2 equiv) and the reaction mixture was stirred at rt for 4 h. After completion of the reaction, the reaction mixture was concentrated under reduced pressure to 100 mL. The residue was diluted with water (10V) and the aqueous layer washed with ethyl acetate (2×2V). The aqueous phase was neutralized with 2N aq HCl solution and extracted with 10% Methanol/DCM (3×5V). The organic layer was dried over sodium sulfate and concentrated to afford crude product. Then the crude product was taken up with 10% IPA/Toluene (10V) solution and treated with activated carbon. The solvent was then removed under reduced pressure. The residue was taken in up in a mixture of 30% Toluene/hexane, stirred for 30 min and filtered, and then dried under vacuum. This process was repeated two additional times to afford Intermediate 1.4. LC/MS: 238.1 [M−H]⁻.

Intermediate 1 was obtained as the first eluent of SFC purification of Intermediate 1.4 using a Chiralpak AD-H column with 20% methanol as co-solvent. LC/MS: 238.1 [M−H]⁻.

### Preparation of Intermediate 2:

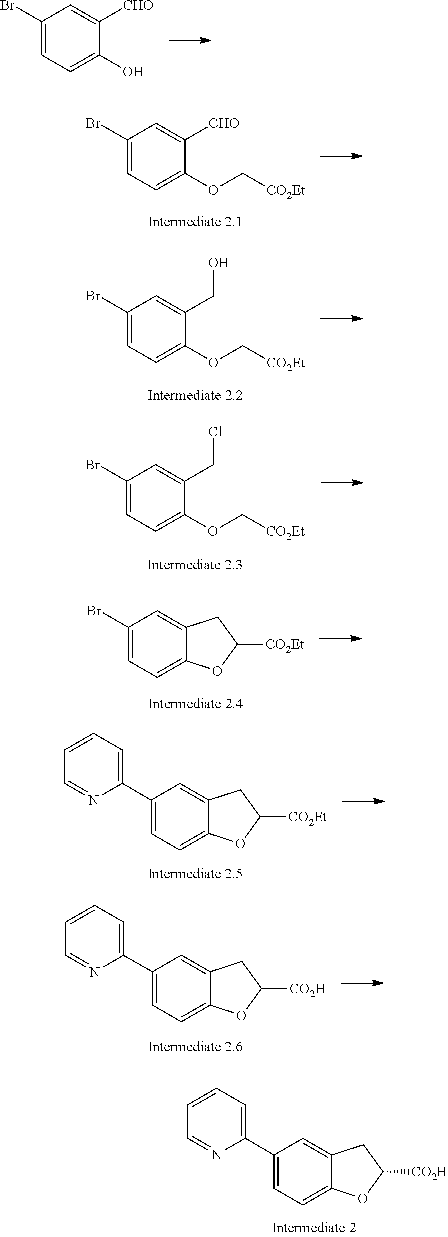

Preparation of Intermediate 2.1: To a stirred solution of 5-bromo-2-hydroxybenzaldehyde (201 g, 1 mol) in DMF (10 V) was added K₂CO₃ (165 g, 1.2 equiv) followed by ethyl bromoacetate (183 g, 1.1 equiv) at 0-15° C. under N₂ atmosphere. The resulting mixture was stirred at room temperature for 20 h at which time it was filtered and the solid washed with EtOAc. The combined filtrate was quenched with ice cold water and extracted with EtOAc. The combined organic layer was washed with water and brine solution, dried over sodium sulfate and concentrated via distillation to afford Intermediate 2.1 as a yellow solid which was used in the next step without any further purification. LC/MS: 285.0 [M−H]⁻.

Preparation of Intermediate 2.2: To a stirred solution of Intermediate 2.1 (150 g, 0.52 mol) in THF (10 V) under N₂ atmosphere was added NaBH₄ (26 g, 1.3 equiv) portion wise at 0° C. The reaction mixture was warmed to room temperature for 2 h, at which time ice water was added, and the mixture was extracted with EtOAc. The combined organic fractions were washed with water and brine solution, dried over sodium sulfate and concentrated via distillation to afford crude product. The crude product was triturated with n-pentane to afford compound Intermediate 2.2 which was used in the next step without further purification. LC/MS: 287.0 [M−H]⁻.

Preparation of Intermediate 2.3: To a stirred solution of Intermediate 2.2 (175 g, 0.61 mol) in DCM (10 V) under N₂ atmosphere was added SOCl₂ (66 mL, 1.5 equiv) at 0° C. The reaction mixture was stirred at room temperature for 2 h at which time it was quenched with saturated aqueous NaHCO₃ solution and extracted with DCM. The combined organic layer was washed with brine solution, dried over anhydrous sodium sulfate and concentrated via distillation to afford crude product. The crude product was purified by column chromatography (silica gel: 100-200 mesh; Eluent: 2-3% EA in PE) to afford Intermediate 2.3. LC/MS: 305.0 [M−H]⁻.

Preparation of Intermediate 2.4: To a stirred solution of Intermediate 2.3 (150 g, 380 mmol) in THF (30 V) under N₂ atmosphere was added t-BuOK (76.6 g, 1.4 equiv) in one portion at 0° C. The reaction mixture was stirred at room temperature for 2 h, at which time it was quenched with saturated aqueous NH₄Cl and extracted with EtOAc. The combined organic layer was washed with brine solution, dried over anhydrous sodium sulfate and concentrated via distillation to afford crude product. The crude product was purified by column chromatography (Silica gel: 100-200 mesh; Eluent: ˜0-5% EA in PE) to afford Intermediate 2.4. LC/MS: 269.0 [M−H]⁻.

Preparation of Intermediate 2.5: To a degassed solution of Intermediate 2.4 (40 g, 148 mmol) in toluene (12 V) at RT was added 2-(tributylstannyl)pyridine, followed by Pd(PPh₃)₄ at RT. The resulting solution was again degassed using argon for 2 min. The reaction mixture was heated to 80° C. for 16 h at which time it was cooled to RT, diluted with EtOAc, stirred for 30 min, and filtered through a Celite pad. The Celite pad was washed with EtOAC twice. The combined filtrate was concentrated under vacuum via distillation to afford crude product. The crude product was purified by column chromatography (silica gel: 100-200 mesh; Eluent: 5% EA in PE) to afford Intermediate 2.5. LC/MS: 270.1 [M+H]⁺.

Preparation of Intermediate 2.6: To a stirred solution of Intermediate 2.5 (45 g, 167 mmol) in mixture of THF and H₂O (10V, ˜1:1 ratio) at RT was added LiOH·H₂O (21.1 g, 3 equiv). The reaction mixture was stirred for 2 h at rt at which time the THF was evaporated from the reaction mixture and the remaining aqueous layer was washed with EtOAc twice. The aqueous layer was acidified with conc. HCl to pH ˜4-5 and stirred for 30 min at 0-10° C. The solid that precipitated was filtered and dried under reduced pressure to afford a hydrochloride salt Intermediate 2.6. LC/MS: 240.1 [M−H]⁻.

Intermediate 2 was obtained as the first eluent from SFC separation employing ChiralART Cellulose SZ(C₂) (250×30×5 μm) column with 30% (0.5% dimethylamine in methanol) as co-solvent.

### Preparation of Intermediate 3:

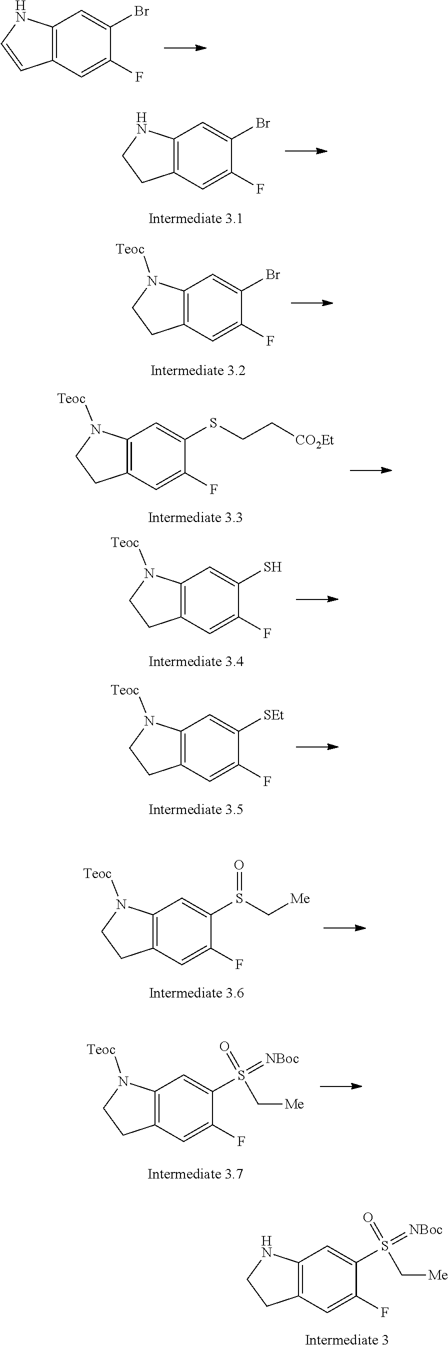

Preparation of Intermediate 3.1: To a solution of 6-bromo-5-fluoro-1H-indole (15 g, 70.1 mmol, 1 equiv) in AcOH (150 mL) was added sodium cyanoborohydride (8.8 g, 140.2 mmol, 2 equiv) under N₂ atmosphere at 0° C. Then the reaction mixture was warmed to 25° C. and stirred for 1 h. The reaction was diluted with water (100 mL), neutralized with NaOH to pH 8, extracted with EtOAc (100 mL×2), and the combined organic phase washed with brine (150 mL), dried over Na₂SO₄ and contracted under vacuum to provide a residue. The residue was dissolved in DCM (60 mL) and 30 g of silica gel was added. The resultant mixture was concentrated under reduced pressure to give a flowing solid, and then submitted for purification by flash chromatography on silica gel (10-15% ethyl acetate in petroleum ether) to afford Intermediate 3.1. LC/MS: 216.0 [M+H]⁺.

Preparation of Intermediate 3.2: To a solution Intermediate 3.1 (13 g, 60.2 mmol, 1 equiv) in DCM (150 mL) was added (2, 5-dioxopyrrolidin-1-yl) 2-trimethylsilylethyl carbonate (23.4 g, 90.3 mmol, 1.5 equiv) and triethylamine (18.3 g, 180.5 mmol, 25.1 mL, 3 equiv) at 0° C. The reaction was stirred at 25° C. for 22 h. The reaction mixture was diluted with water (150 mL), extracted with ethyl acetate (150 mL×2). The combined organic phase was washed with brine (100 mL), dried over Na₂SO₄, filtered and concentrated under reduced pressure to provide a residue. The residue was dissolved in DCM (100 mL) and 26 g of silica gel was added. The resultant mixture was concentrated under reduced pressure to give a flowing solid, and then submitted for purification by flash chromatography on silica gel (10-15% ethyl acetate in petroleum ether) to afford Intermediate 3.2. LC/MS: 360.0 [M+H]⁺.

Preparation of Intermediate 3.3: To a solution of Intermediate 3.2 (6.5 g, 18.0 mmol, 1 equiv) in dioxane (70 mL) was added DIPEA (4.7 g, 36.1 mmol, 6.3 mL, 2 equiv), Xantphos (1.0 g, 1.8 mmol, 0.1 equiv), Pd₂(dba)₃ (826.0 mg, 902.0 umol, 0.05 equiv) and ethyl 3-sulfanylpropanoate (7.3 g, 54.1 mmol, 3 equiv). The reaction mixture was stirred at 100° C. for 16 h. The reaction mixture was diluted with water (70 mL) and extracted with ethyl acetate (50 mL×2). The combined organic phase was washed with brine (70 mL), dried over Na₂SO₄, filtered and concentrated under reduced pressure to afford a residue. The residue was dissolved in DCM (70 mL) and 13 g of silica gel was added. The resultant mixture was concentrated under reduced pressure to give a flowing solid and then submitted for purification by flash chromatography on silica gel (5-10% ethyl acetate in petroleum ether) to afford Intermediate 3.3. LC/MS: 414.1 [M+H]⁺.

Preparation of Intermediate 3.5: To a solution of Intermediate 3.3 (2 g, 4.8 mmol, 1 equiv) in THF (20 mL) was added t-BuOK (814.0 mg, 7.3 mmol, 1.5 equiv) at 0° C. under N₂ atmosphere and the mixture stirred at 25° C. for 1 h, which afforded crude Intermediate 3.4. In the same flask, iodoethane (2.3 g, 14.5 mmol, 1.2 mL, 3 equiv) was added to the mixture at 0° C. under N₂ atmosphere and the solution stirred at 25° C. for 15 h. The reaction mixture was diluted with water (20 mL) and extracted with ethyl acetate (25 mL×2). The combined organic phase was washed with brine (20 mL), dried over Na₂SO₄, filtered and concentrated under reduced pressure to afford a residue. The residue was dissolved in DCM (20 mL) and 2 g of silica gel was added. The resultant mixture was concentrated under reduced pressure to give a flowing solid and then submitted for purification by flash chromatography on silica gel (0-5% ethyl acetate in petroleum ether) to afford Intermediate 3.5. LC/MS: 342.1 [M+H]⁺.

Preparation of Intermediate 3.6: To a solution of Intermediate 3.5 (1 g, 2.9 mmol, 1 equiv) in DCM (10 mL) at −30° C., was added m-CPBA (653.9 mg, 3.2 mmol, 85% purity, and 1.1 equiv). After stirring at −30° C. for 1 h, the reaction mixture was quenched with saturated Na₂SO₃ aqueous solution (30 mL) and extracted with DCM (10 mL×2). The combined organic phase was washed with saturated NaHCO₃ aqueous solution (10 mL) and brine (10 mL), dried over Na₂SO₄, filtered and concentrated under reduced pressure to afford Intermediate 3.6. LC/MS: 358.1 [M+H]⁺.

Preparation of Intermediate 3.7: To a solution of Intermediate 3.6 (1.3 g, 3.5 mmol, 1 equiv) in DCM (15 mL) was added diacetoxyrhodium (92. mg, 209.8 mol, 0.06 equiv), MgO (1.1 g, 28.0 mmol, 314.9 μL, 8 equiv) and tert-butyl carbamate (1.2 g, 10.5 mmol, 3 equiv). Then PIDA (4.5 g, 14.0 mmol, 4 equiv) was added dropwise to the mixture which was stirred at 20° C. for 12 h. The reaction was filtered, and the filtrate was quenched with saturated (aq.) Na₂SO₃ (20 mL) and extracted with DCM (20 mL×2). The combined organic phase was washed with brine (20 mL), dried over Na₂SO₄, filtered, and concentrated under reduced pressure to afford a residue. The residue was dissolved in DCM (20 mL) and 1 g of silica gel was added. The resultant mixture was concentrated under reduced pressure to give a flowing solid, and then submitted for purification by flash chromatography on silica gel (15-20% ethyl acetate in petroleum ether) to afford Intermediate 3.7. LC/MS: 473.2 [M+H]⁺.

Preparation of Intermediate 3: To a solution of Intermediate 3.7 (400 mg, 846.3 μmol, 1 equiv) in THF (4 mL) was added TBAF (1 M, 4.2 mL, 5 equiv) and the mixture was stirred at 50° C. for 2 h. The reaction mixture was diluted with water (5 mL) and extracted with ethyl acetate (5 mL×2). The combined organic phase was washed with brine (5 mL), dried over Na₂SO₄, filtered and concentrated under reduced pressure to give a residue. The residue was purified by flash column chromatography (10-50% ethyl acetate in petroleum ether) to afford Intermediate 3. LC/MS: 329.1 [M+H]⁺.

The following intermediates were prepared in a manner similar to Intermediate 3, employing the appropriate indolines and alkyl halides for sulfur alkylation as required.

| Compound: | Structure: | LC/MS: |
|---|---|---|
| Intermediate 4 | 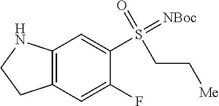 | 343.1 [M + H]⁺ |
| Intermediate 5 | 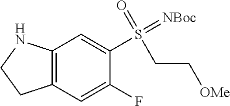 | 359.1 [M + H]⁺ |
| Intermediate 6 | 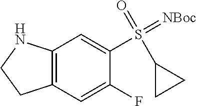 | 341.1 [M + H]⁺ |
| Intermediate 7 | 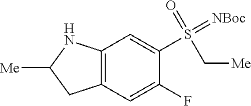 | 343.1 [M + H]⁺ |

### Preparation of Intermediate 8:

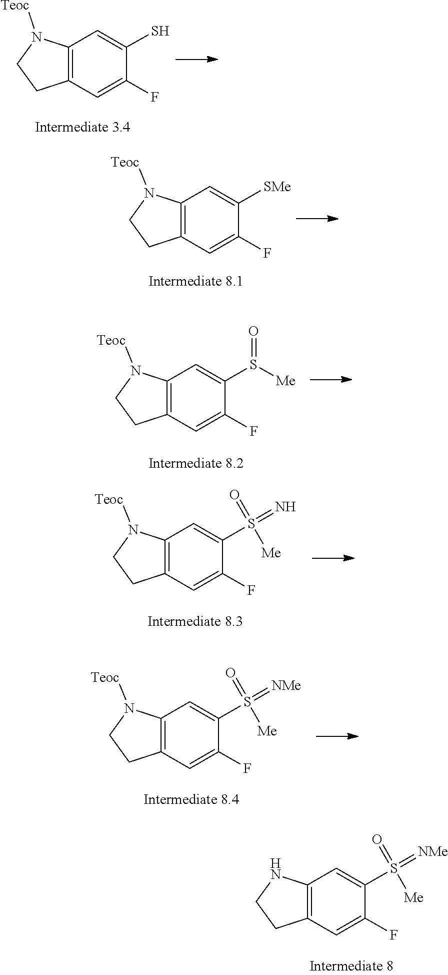

Preparation of Intermediate 8.1: To a solution of Intermediate 3.3 (2 g, 4.8 mmol, 1 eq) in THF (20 mL) was added t-BuOK (813.9 mg, 7.2 mmol, 1.5 eq) at 0° C. under N₂ atmosphere and the mixture was stirred at 20° C. for 1 h to afford Intermediate 3.4. Then Mel (2.0 g, 14.5 mmol, 903.1 μL, 3 eq) was added to the mixture at 0° C. under N₂ atmosphere and the mixture was stirred at 20° C. for 15 h. LCMS showed the starting material was consumed completely and ˜72% of a compound having the desired molecular weight was detected. The reaction mixture was diluted with water (20 mL) extracted with ethyl acetate (20 mL×2). The combined organic phase was washed with brine (20 mL), dried over Na₂SO₄, filtered and concentrated under reduced pressure to afford Intermediate 8.1, which was used in the next step without further purification. LC/MS: 328.1 [M+H]⁺.

Preparation of Intermediate 8.3 was achieved starting from Intermediate 8.1 employing methodology used in the transformation of Intermediate 3.5 to Intermediate 3.7. LC/MS: 459.2 [M+H]⁺.

Preparation of Intermediate 8.4: To a solution of Intermediate 8.3 (630 mg, 1.7 mmol, 1 eq) in THF (8 mL) was added NaH (140.5 mg, 3.5 mmol, 60% in mineral oil, 2 eq) under N₂ atmosphere at 0° C. and the mixture was stirred at 0° C. for 1 h. Then iodomethane (1.25 g, 8.8 mmol, 546.9 μL, 5 eq) was added and the mixture was stirred at 20° C. for 15 h. The reaction mixture was quenched with saturated (aq.) NH₄Cl (20 mL) and extracted with ethyl acetate (10 mL×3). The combined organic phase was washed with brine (10 mL), dried over Na₂SO₄, filtered and concentrated under reduced pressure to afford Intermediate 8.4, which was used in the next step without further purification. LC/MS: 373.1 [M+H]⁺.

Intermediate 8 was obtained in a manner similar to Intermediate 3, employing Intermediate 8.4 as starting material. LC/MS: 229.1 [M+H]⁺.

### Preparation of Intermediate 9:

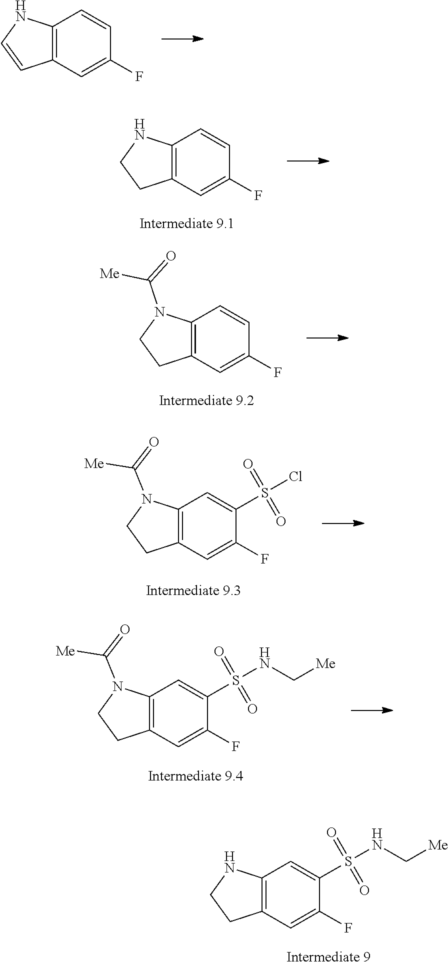 [SMILES: Fc1ccc2[nH]ccc2c1 ; Fc1ccc2c(c1)CCN2 ; CC(=O)N1CCc2cc(F)ccc21 ; CC(=O)N1CCc2cc(F)c(S(=O)(=O)Cl)cc21 ; CCNS(=O)(=O)c1cc2c(cc1F)CCN2C(C)=O ; CCNS(=O)(=O)c1cc2c(cc1F)CCN2]

Preparation of Intermediate 9.1: To a solution of 5-fluoro-1H-indole (25 g, 185.0 mmol, 1 eq.) in AcOH (220 mL) was added sodium cyanoborohydride (23.2 g, 369.9 mmol, 2 eq.) at 0° C. under N₂. Then the mixture was stirred at 20° C. for 16 h. The reaction was diluted with H₂O (200 mL) and neutralized with saturated (aq.) NaHCO₃ to pH 7, then extracted with EtOAc (150 mL×2) The combined organic phase was washed with brine (150 mL), dried over Na₂SO₄ and concentrated under vacuum to afford Intermediate 9.1, which was used in the next step without further purification. LC/MS: 138.1 [M+H]⁺.

Preparation of Intermediate 9.2: To a solution of Intermediate 9.1 (12.5 g, 91.1 mmol, 1 eq.) in DCM (200 mL) was added TEA (27.6 g, 273.4 mmol, 38.1 mL, 3 eq). Acetyl chloride (14.3 g, 182.3 mmol, 12.9 mL, 2 eq) was added to the mixture at 0° C. and the mixture was stirred at 20° C. for 1 h. The reaction mixture was diluted with water (150 mL) and extracted with DCM (200 mL×2). The combined organic phase was washed with brine (150 mL), dried over Na₂SO₄, filtered and concentrated under reduced pressure to afford Intermediate 9.2, which was used directly in the next step without further purification. LC/MS: 180.1 [M+H]⁺.

Preparation of Intermediate 9.3: A solution of Intermediate 9.2 (6 g, 33.5 mmol, 1 eq) in HSO₃Cl (84.1 g, 722.1 mmol, 48 mL, 21.57 eq) was stirred at 50° C. for 16 h. The reaction mixture was cooled to room temperature and slowly quenched with water (100 mL), filtered and the filter cake rinsed with H₂O (20 mL×3) followed by petroleum ether (20 mL×3) to afford Intermediate 9.3. LC/MS: 278.0 [M+H]⁺.

Preparation of Intermediate 9.4: A solution of Intermediate 9.3 (2 g, 7.2 mmol, 1 eq) in ethylamine (2 M, 18.1 mL, 5 eq) was stirred at 20° C. for 1 h. The mixture was concentrated under reduced pressure to afford Intermediate 9.4. LC/MS: 287.1 [M+H]⁺.

Preparation of Intermediate 9: To a solution of Intermediate 9.4 (1 g, 3.5 mmol, 1 eq) in EtOH (10 mL) was added HCl (12 M, 2 mL, 6.9 eq). The mixture was stirred at 80° C. for 3 h. The mixture was concentrated under reduced pressure to afford Intermediate 9. LC/MS: 245.3 [M+H]⁺.

### Preparation of Example 1 (ethyl(5-fluoro-1-((R)-5-(pyridin-2-yl)-2,3-dihydro-1H-indene-2-carbonyl)indolin-6-yl)(imino)-λ⁶-sulfanone)

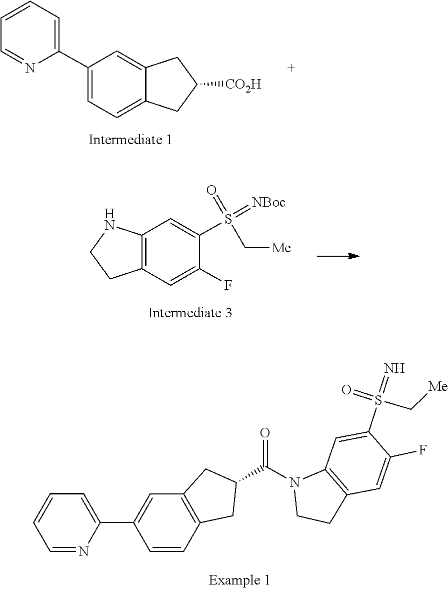

Example 1 was prepared using General Procedure A, employing Intermediate 1 and Intermediate 3 as starting materials. ¹H NMR (400 MHz, DMSO-d₆): δ 8.70-8.50 (m, 2H), 7.98-7.80 (m, 4H), 7.39-7.27 (m, 3H), 4.54 (s, 1H), 4.33 (br t, J=8.6 Hz, 2H), 3.73 (br t, J=8.1 Hz, 1H), 3.32-3.18 (m, 8H), 1.08 (t, J=7.4 Hz, 3H) ppm. LC/MS: 450.2 [M+H]⁺.

### Preparation of Example 2 ((R)-ethyl(5-fluoro-1-((R)-5-(pyridin-2-yl)-2,3-dihydro-1H-indene-2-carbonyl)indolin-6-yl)(imino)-λ⁶-sulfanone)

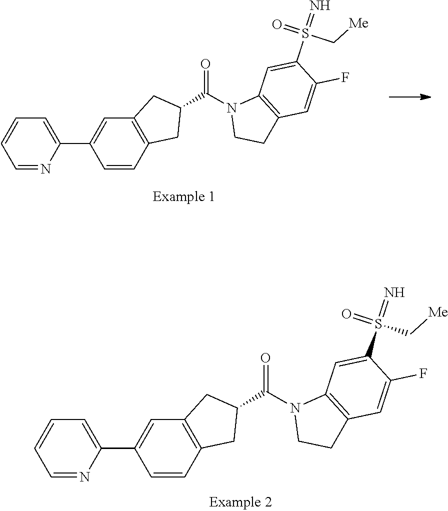 [SMILES: CCS(=N)(=O)c1cc2c(cc1F)CCN2C(=O)[C@@H]1Cc2ccc(-c3ccccn3)cc2C1 ; CC[S@@](=N)(=O)c1cc2c(cc1F)CCN2C(=O)[C@@H]1Cc2ccc(-c3ccccn3)cc2C1]

Example 2 was collected as the first eluent from chiral SFC separation of Example 1 using ChiralPak IH, 250×30 mm, 10 μm; mobile phase: 50% [CO₂-MeOH(0.1% NH₃—H₂O)]. ¹H NMR (400 MHz, DMSO-d₆): δ 8.70-8.52 (m, 2H), 8.02-7.78 (m, 4H), 7.41-7.27 (m, 3H), 4.54 (s, 1H), 4.34 (br t, J=8.6 Hz, 2H), 3.73 (t, J=8.0 Hz, 1H), 3.31-3.24 (m, 6H), 3.23-3.18 (m, 2H), 1.08 (t, J=7.3 Hz, 3H) ppm. LC/MS: 450.1 [M+H]⁺.

### Preparation of Example 3 ((S)-ethyl(5-fluoro-1-((R)-5-(pyridin-2-yl)-2,3-dihydro-1H-indene-2-carbonyl)indolin-6-yl)(imino)-λ⁶-sulfanone)

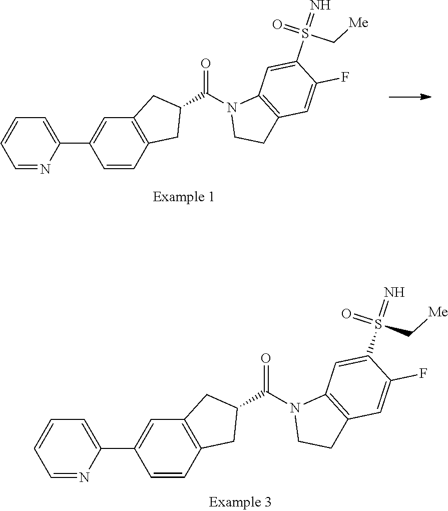 [SMILES: CCS(=N)(=O)c1cc2c(cc1F)CCN2C(=O)[C@@H]1Cc2ccc(-c3ccccn3)cc2C1 ; CC[S@](=N)(=O)c1cc2c(cc1F)CCN2C(=O)[C@@H]1Cc2ccc(-c3ccccn3)cc2C1]

Example 3 was collected as the second eluent from chiral SFC separation of Example 1 using ChiralPak IH, 250×30 mm, 10 μm; mobile phase: 50% [CO₂-MeOH(0.1% NH₃—H₂O)]. ¹H NMR (400 MHz, DMSO-d₆): δ 8.71-8.50 (m, 2H), 8.04-7.75 (m, 4H), 7.45-7.21 (m, 3H), 4.54 (s, 1H), 4.34 (br t, J=8.5 Hz, 2H), 3.73 (br s, 1H), 3.35-3.25 (m, 2H), 3.20 (br d, J=7.3 Hz, 2H), 1.08 (t, J=7.3 Hz, 3H) ppm. LC/MS: 450.0 [M+H]⁺.

### Preparation of Example 4 (ethyl(5-fluoro-1-((R)-5-(pyridin-2-yl)-2,3-dihydrobenzofuran-2-carbonyl)indolin-6-yl)(imino)-λ⁶-sulfanone)

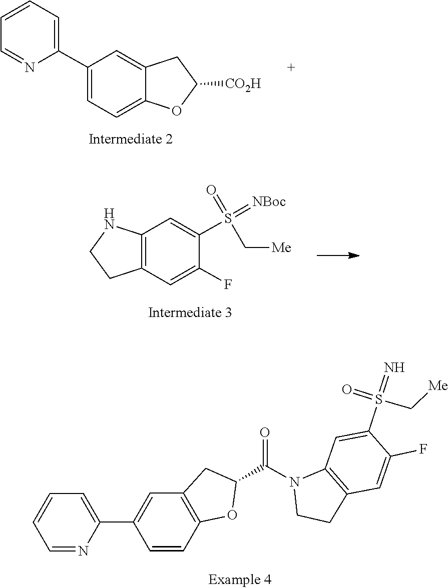

Example 4 was prepared using General Procedure A, employing Intermediate 2 and Intermediate 3 as starting materials. ¹H NMR (400 MHz, DMSO-d6): δ 8.66-8.48 (m, 2H), 8.01-7.80 (m, 4H), 7.42-7.25 (m, 2H), 6.94 (dd, J=3.4, 8.4 Hz, 1H), 5.81-5.70 (m, 1H), 4.57 (s, 1H), 4.46-4.26 (m, 2H), 3.66-3.50 (m, 2H), 3.30 (br s, 2H), 3.20 (q, J=7.3 Hz, 2H), 1.07 (t, J=7.4 Hz, 3H) ppm. LC/MS: 452.0 [M+H]⁺.

### Preparation of Example 5 ((S)-ethyl(5-fluoro-1-((R)-5-(pyridin-2-yl)-2,3-dihydrobenzofuran-2-carbonyl)indolin-6-yl)(imino)-λ⁶-sulfanone)

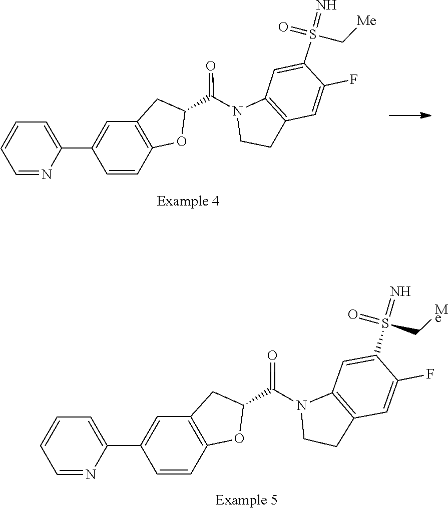 [SMILES: CCS(=N)(=O)c1cc2c(cc1F)CCN2C(=O)[C@H]1Cc2cc(-c3ccccn3)ccc2O1 ; CC[S@](=N)(=O)c1cc2c(cc1F)CCN2C(=O)[C@H]1Cc2cc(-c3ccccn3)ccc2O1]

Example 5 was obtained as the first eluent from chiral SFC separation of Example 4 using REGIS(S,S)WHELK-O1 (250 mm×25 mm, 10 μm) column; 75% EtOH as co-solvent. ¹H NMR (400 MHz, DMSO-d₆): δ 8.68-8.45 (m, 2H), 8.05-7.79 (m, 4H), 7.43-7.23 (m, 2H), 6.94 (d, J=8.5 Hz, 1H), 5.75 (t, J=8.0 Hz, 1H), 4.56 (s, 1H), 4.46-4.28 (m, 2H), 3.59 (br d, J=8.3 Hz, 2H), 3.32-3.28 (m, 2H), 3.20 (q, J=7.4 Hz, 2H), 1.08 (t, J=7.3 Hz, 3H) ppm. LC/MS: 452.0 [M+H]⁺.

### Preparation of Example 6 ((R)-ethyl(5-fluoro-1-((R)-5-(pyridin-2-yl)-2,3-dihydrobenzofuran-2-carbonyl)indolin-6-yl)(imino)-λ⁶-sulfanone)

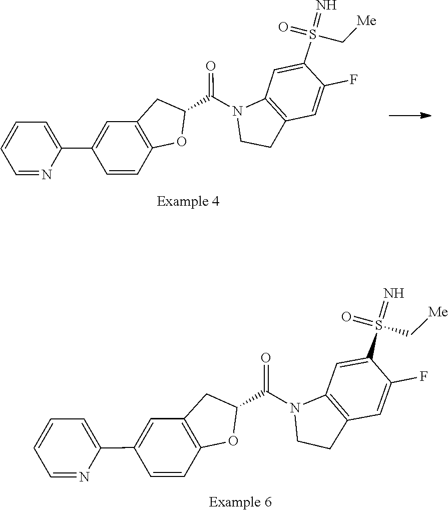 [SMILES: CCS(=N)(=O)c1cc2c(cc1F)CCN2C(=O)[C@H]1Cc2cc(-c3ccccn3)ccc2O1 ; CC[S@@](=N)(=O)c1cc2c(cc1F)CCN2C(=O)[C@H]1Cc2cc(-c3ccccn3)ccc2O1]

Example 6 was obtained as the second eluent from chiral SFC separation of Example 4 using REGIS(S,S)WHELK-O1 (250 mm×25 mm, 10 μm) column; 75% EtOH as co-solvent. ¹H NMR (400 MHz, DMSO-d₆): δ 8.70-8.44 (m, 2H), 8.02-7.79 (m, 4H), 7.42-7.24 (m, 2H), 6.95 (d, J=8.5 Hz, 1H), 5.75 (dd, J=6.4, 9.8 Hz, 1H), 4.56 (s, 1H), 4.46-4.26 (m, 2H), 3.67-3.51 (m, 2H), 3.33-3.27 (m, 2H), 3.21 (q, J=7.4 Hz, 2H), 1.07 (t, J=7.4 Hz, 3H) ppm. LC/MS: 452.0 [M+H]⁺.

### Preparation of Example 7 ((5-fluoro-1-((R)-5-(pyridin-2-yl)-2,3-dihydro-1H-indene-2-carbonyl)indolin-6-yl)(imino)(propyl)-λ⁶-sulfanone)

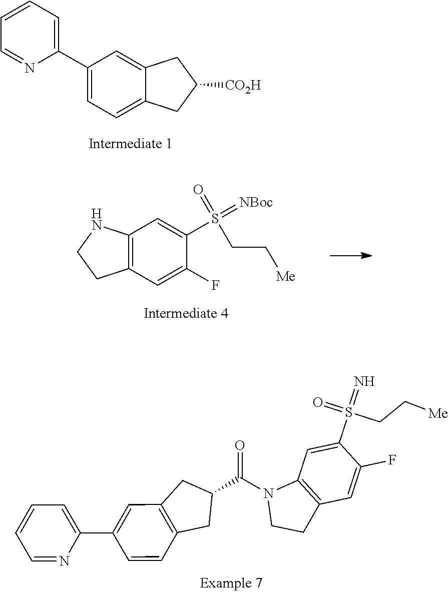

Example 7 was prepared using General Procedure A, employing Intermediate 1 and Intermediate 4 as starting materials. ¹H NMR (400 MHz, DMSO-d₆): δ 8.56 (td, J=1.3, 4.8 Hz, 1H), 8.48 (d, J=6.6 Hz, 1H), 7.93-7.80 (m, 3H), 7.77 (d, J=7.9 Hz, 1H), 7.36-7.26 (m, 3H), 4.28 (br t, J=8.5 Hz, 2H), 3.67 (br t, J=7.8 Hz, 1H), 3.37-3.13 (m, 8H), 1.57-1.44 (m, 2H), 0.84 (t, J=7.4 Hz, 3H) ppm. LC/MS: 464.2 [M+H]⁺.

### Preparation of Example 8 ((R)-(5-fluoro-1-((R)-5-(pyridin-2-yl)-2,3-dihydro-1H-indene-2-carbonyl)indolin-6-yl)(imino)(propyl)-λ⁶-sulfanone)

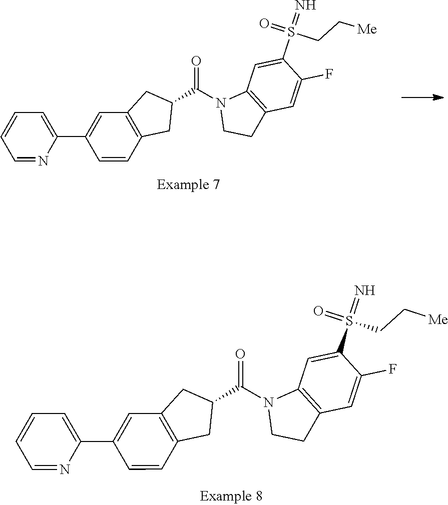 [SMILES: CCCS(=N)(=O)c1cc2c(cc1F)CCN2C(=O)[C@@H]1Cc2ccc(-c3ccccn3)cc2C1 ; CCC[S@@](=N)(=O)c1cc2c(cc1F)CCN2C(=O)[C@@H]1Cc2ccc(-c3ccccn3)cc2C1]

Example 8 was obtained as the first eluent from chiral SFC purification of Example 7 using ChiralPak IH (250×30 mm×10 μm); 50% co-solvent (0.1% NH₃ in methanol). ¹H NMR (400 MHz, DMSO-d₆): δ 8.66-8.56 (m, 2H), 7.95-7.83 (m, 4H), 7.37-7.30 (m, 3H), 4.52 (s, 1H), 4.34 (br t, J=8.4 Hz, 2H), 3.73 (t, J=8.1 Hz, 1H), 3.37-3.25 (m, 6H), 3.20-3.15 (m, 2H), 1.60-1.51 (m, 2H), 0.89 (t, J=7.4 Hz, 3H) ppm. LC/MS: 464.2 [M+H]⁺.

### Preparation of Example 9 ((S)-(5-fluoro-1-((R)-5-(pyridin-2-yl)-2,3-dihydro-1H-indene-2-carbonyl)indolin-6-yl)(imino)(propyl)-λ⁶-sulfanone)

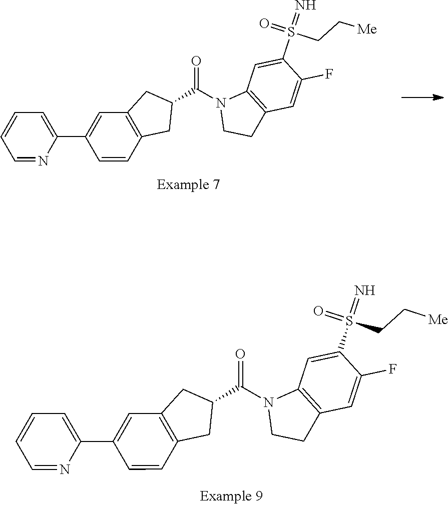 [SMILES: CCCS(=N)(=O)c1cc2c(cc1F)CCN2C(=O)[C@@H]1Cc2ccc(-c3ccccn3)cc2C1 ; CCC[S@](=N)(=O)c1cc2c(cc1F)CCN2C(=O)[C@@H]1Cc2ccc(-c3ccccn3)cc2C1]

Example 9 was obtained as the second eluent from chiral SFC purification of Example 7 using ChiralPak IH (250×30 mm×10 μm); 50% co-solvent (0.1% NH₃ in methanol). ¹H NMR (400 MHz, DMSO-d₆): δ 8.67-8.56 (m, 2H), 7.96-7.83 (m, 4H), 7.37-7.30 (m, 3H), 4.34 (br t, J=8.6 Hz, 2H), 3.73 (quin, J=7.9 Hz, 1H), 3.35-3.25 (m, 6H), 3.20-3.16 (m, 2H), 1.59-1.51 (m, 2H), 0.89 (t, J=7.4 Hz, 3H) ppm. LC/MS: 464.2 [M+H]⁺.

### Preparation of Example 10 ((5-fluoro-1-((R)-5-(pyridin-2-yl)-2,3-dihydrobenzofuran-2-carbonyl)indolin-6-yl)(imino)(propyl)-λ⁶-sulfanone)

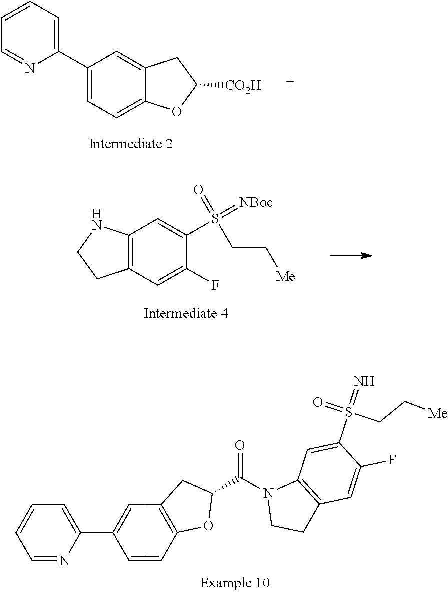

Example 10 was prepared using General Procedure A, employing Intermediate 2 and Intermediate 4 as starting materials. ¹H NMR (400 MHz, DMSO-d₆): δ 8.63-8.40 (m, 2H), 7.93-7.74 (m, 4H), 7.41-7.21 (m, 2H), 7.01-6.87 (m, 1H), 5.68 (dd, J=5.8, 10.0 Hz, 1H), 4.39-4.20 (m, 2H), 3.66-3.42 (m, 2H), 3.34-3.15 (m, 4H), 1.58-1.42 (m, 2H), 0.87-0.80 (m, 3H) ppm. LC/MS: 466.2 [M+H]⁺.

### Preparation of Example 11 ((S)-(5-fluoro-1-((R)-5-(pyridin-2-yl)-2,3-dihydrobenzofuran-2-carbonyl)indolin-6-yl)(imino)(propyl)-λ⁶-sulfanone)

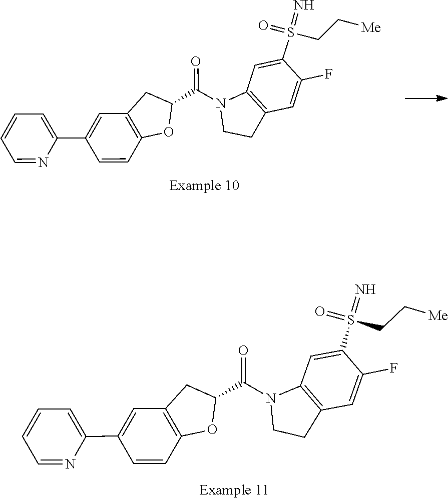 [SMILES: CCCS(=N)(=O)c1cc2c(cc1F)CCN2C(=O)[C@H]1Cc2cc(-c3ccccn3)ccc2O1 ; CCC[S@](=N)(=O)c1cc2c(cc1F)CCN2C(=O)[C@H]1Cc2cc(-c3ccccn3)ccc2O1]

Example 11 was obtained as the first eluent from chiral SFC purification of Example 10, using DAICEL CHIRALCEL OJ (250 mm×30 mm, 10 um) column; 50% (0.1% NH₃ in methanol) as co-solvent. ¹H NMR (400 MHz, DMSO-d₆): δ 8.64-8.49 (m, 2H), 8.06-7.76 (m, 4H), 7.43-7.23 (m, 2H), 6.94 (d, J=8.4 Hz, 1H), 5.75 (t, J=8.1 Hz, 1H), 4.55 (s, 1H), 4.46-4.27 (m, 2H), 3.59 (br d, J=8.0 Hz, 2H), 3.31-0.3.25 (m, 2H), 3.21-3.15 (m, 2H), 1.61-1.47 (m, 2H), 0.88 (t, J=7.4 Hz, 3H) ppm. LC/MS: 466.0 [M+H]⁺.

### Preparation of Example 12 ((R)-(5-fluoro-1-((R)-5-(pyridin-2-yl)-2,3-dihydrobenzofuran-2-carbonyl)indolin-6-yl)(imino)(propyl)-λ⁶-sulfanone)

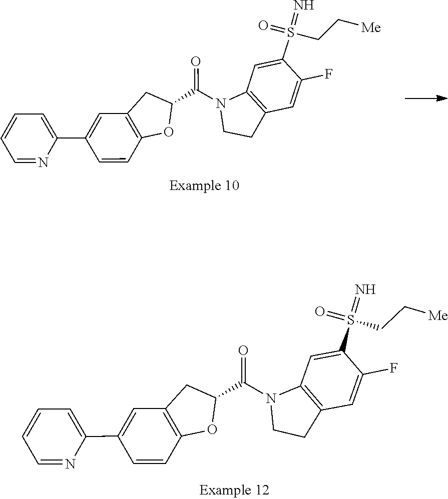 [SMILES: CCCS(=N)(=O)c1cc2c(cc1F)CCN2C(=O)[C@H]1Cc2cc(-c3ccccn3)ccc2O1 ; CCC[S@@](=N)(=O)c1cc2c(cc1F)CCN2C(=O)[C@H]1Cc2cc(-c3ccccn3)ccc2O1]

Example 12 was obtained as the second eluent from chiral SFC purification of Example 10, using DAICEL CHIRALCEL OJ (250 mm×30 mm, 10 um) column; 50% (0.1% NH₃ in methanol) as co-solvent. ¹H NMR (400 MHz, DMSO-d₆): δ 8.63-8.49 (m, 2H), 8.02-7.79 (m, 4H), 7.43-7.21 (m, 2H), 6.94 (d, J=8.5 Hz, 1H), 5.75 (dd, J=6.4, 9.8 Hz, 1H), 4.56 (s, 1H), 4.47-4.25 (m, 2H), 3.66-3.51 (m, 2H), 3.30 3.31-0.3.25 (m, 2H), 3.22-3.15 (m, 2H), 1.54 (qd, J=7.5, 15.3 Hz, 2H), 0.88 (t, J=7.4 Hz, 3H) ppm. LC/MS: 466.0 [M+H]⁺.

### Preparation of Example 13 ((5-fluoro-1-((R)-5-(pyridin-2-yl)-2,3-dihydro-1H-indene-2-carbonyl)indolin-6-yl)(imino)(2-methoxyethyl)-16-sulfanone)

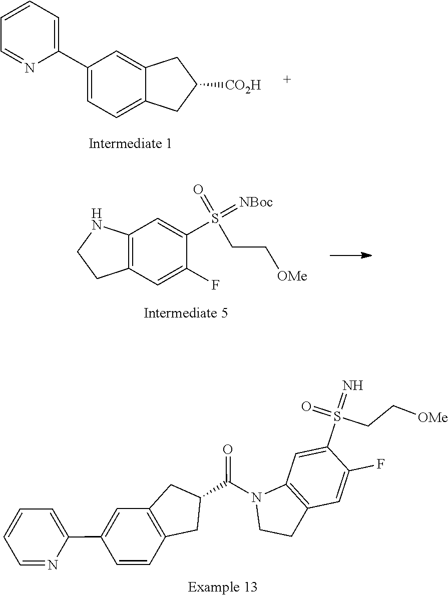

Example 13 was prepared using General Procedure A, employing Intermediate 1 and Intermediate 5 as starting materials. ¹H NMR (400 MHz, DMSO-d₆): δ 8.64 (d, J=4.2 Hz, 1H), 8.57 (d, J=6.6 Hz, 1H), 7.99-7.82 (m, 4H), 7.38-7.29 (m, 3H), 4.66 (s, 1H), 4.34 (br t, J=8.6 Hz, 2H), 3.80-3.68 (m, 1H), 3.66-3.52 (m, 2H), 3.51-3.41 (m, 2H), 3.33-3.19 (m, 6H), 3.08 (s, 3H) ppm. LC/MS: 480.2 [M+H]⁺.

### Preparation of Example 14 ((R)-(5-fluoro-1-((R)-5-(pyridin-2-yl)-2,3-dihydro-1H-indene-2-carbonyl)indolin-6-yl)(imino)(2-methoxyethyl)-λ⁶-sulfanone)

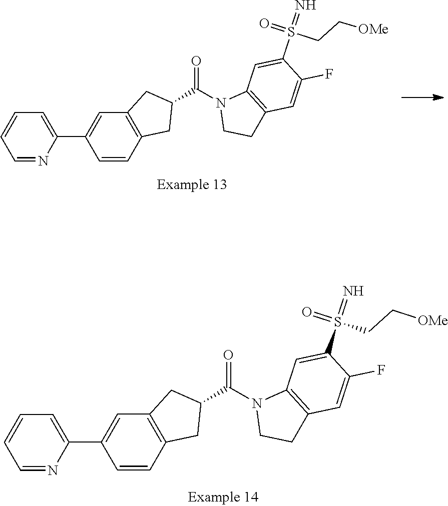 [SMILES: COCCS(=N)(=O)c1cc2c(cc1F)CCN2C(=O)[C@@H]1Cc2ccc(-c3ccccn3)cc2C1 ; COCC[S@@](=N)(=O)c1cc2c(cc1F)CCN2C(=O)[C@@H]1Cc2ccc(-c3ccccn3)cc2C1]

Example 14 was obtained as the first eluent from chiral SFC purification of Example 13 using ChiralPak IH (250×30 mm×10 μm); 50% co-solvent (0.1% NH₃ in methanol). ¹H NMR (400 MHz, DMSO-d₆): δ 8.64 (d, J=4.5 Hz, 1H), 8.57 (d, J=6.6 Hz, 1H), 8.00-7.78 (m, 4H), 7.38-7.28 (m, 3H), 4.66 (s, 1H), 4.34 (br t, J=8.7 Hz, 2H), 3.74 (quin, J=8.0 Hz, 1H), 3.66-3.53 (m, 2H), 3.51-3.44 (m, 2H), 3.33-3.20 (m, 6H), 3.08 (s, 3H) ppm. LC/MS: 480.1 [M+H]⁺.

### Preparation of Example 15 ((S)-(5-fluoro-1-((R)-5-(pyridin-2-yl)-2,3-dihydro-1H-indene-2-carbonyl)indolin-6-yl)(imino)(2-methoxyethyl)-λ6-sulfanone)

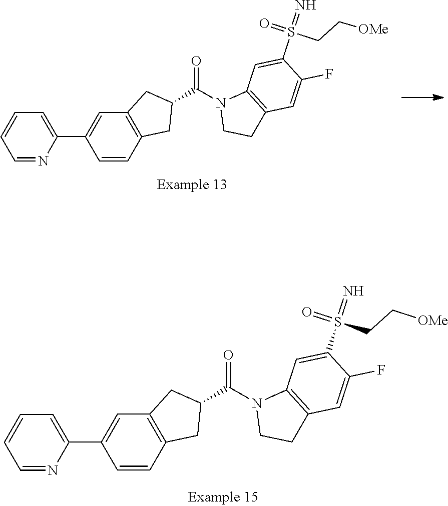 [SMILES: COCCS(=N)(=O)c1cc2c(cc1F)CCN2C(=O)[C@@H]1Cc2ccc(-c3ccccn3)cc2C1 ; COCC[S@](=N)(=O)c1cc2c(cc1F)CCN2C(=O)[C@@H]1Cc2ccc(-c3ccccn3)cc2C1]

Example 15 was obtained as the second eluent from chiral SFC purification of Example 13 using ChiralPak IH (250×30 mm×10 μm); 50% co-solvent (0.1% NH₃ in methanol). ¹H NMR (400 MHz, DMSO-d6): δ 8.64 (d, J=4.5 Hz, 1H), 8.58 (d, J=6.7 Hz, 1H), 7.98-7.81 (m, 4H), 7.37-7.27 (m, 3H), 4.66 (s, 1H), 4.34 (br t, J=8.6 Hz, 2H), 3.74 (quin, J=8.0 Hz, 1H), 3.66-3.52 (m, 2H), 3.51-3.42 (m, 2H), 3.33-3.19 (m, 6H), 3.08 (s, 3H) ppm. LC/MS: 480.1 [M+H]⁺.

### Preparation of Example 16 ((5-fluoro-1-((R)-5-(pyridin-2-yl)-2,3-dihydrobenzofuran-2-carbonyl)indolin-6-yl)(imino)(2-methoxyethyl)-λ⁶-sulfanone)

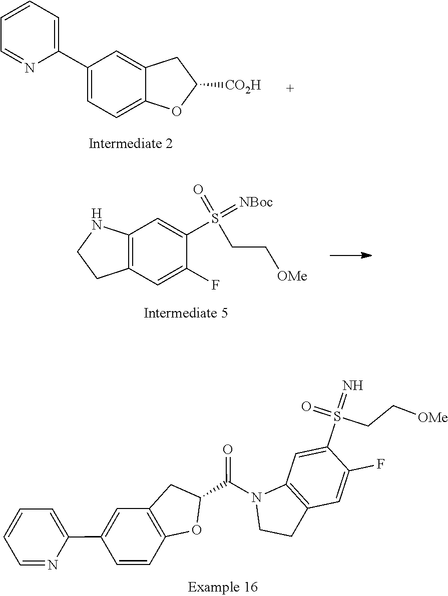

Example 16 was prepared using General Procedure A, employing Intermediate 2 and Intermediate 5 as starting materials. ¹H NMR (400 MHz, DMSO-d6): δ 8.60 (d, J=4.2 Hz, 1H), 8.52 (d, J=6.6 Hz, 1H), 8.00 (s, 1H), 7.93-7.78 (m, 3H), 7.38 (d, J=9.8 Hz, 1H), 7.27 (ddd, J=1.0, 5.5, 6.6 Hz, 1H), 6.94 (dd, J=3.4, 8.4 Hz, 1H), 5.75 (dd, J=7.5, 8.7 Hz, 1H), 4.69 (s, 1H), 4.47-4.24 (m, 2H), 3.65-3.53 (m, 4H), 3.50-3.42 (m, 2H), 3.33-3.27 (m, 2H), 3.06 (s, 3H) ppm. LC/MS: 482.2 [M+H]⁺.

### Preparation of Example 17 ((R)-(5-fluoro-1-((R)-5-(pyridin-2-yl)-2,3-dihydrobenzofuran-2-carbonyl)indolin-6-yl)(imino)(2-methoxyethyl)-λ⁶-sulfanone)

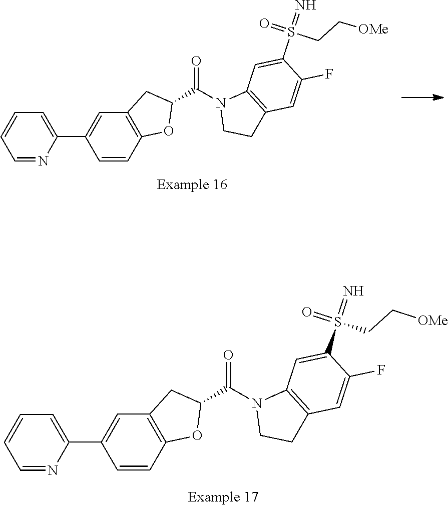 [SMILES: COCCS(=N)(=O)c1cc2c(cc1F)CCN2C(=O)[C@H]1Cc2cc(-c3ccccn3)ccc2O1 ; COCC[S@@](=N)(=O)c1cc2c(cc1F)CCN2C(=O)[C@H]1Cc2cc(-c3ccccn3)ccc2O1]

Example 17 was obtained as the first eluent from chiral SFC purification of Example 16 using DAICEL CHIRALCEL OJ (250 mm×30 mm, 10 um) column; 50% (0.1% NH₃ in methanol) as co-solvent. ¹H NMR (400 MHz, DMSO-d6): δ 8.60 (d, J=4.8 Hz, 1H), 8.52 (d, J=6.7 Hz, 1H), 8.00 (s, 1H), 7.93-7.78 (m, 3H), 7.38 (d, J=9.8 Hz, 1H), 7.27 (ddd, J=1.1, 5.4, 6.6 Hz, 1H), 6.95 (d, J=8.4 Hz, 1H), 5.75 (dd, J=6.4, 9.7 Hz, 1H), 4.68 (s, 1H), 4.46-4.25 (m, 2H), 3.65-3.52 (m, 4H), 3.50-3.43 (m, 2H), 3.33-3.27 (m, 2H), 3.06 (s, 3H) ppm. LC/MS: 482.0 [M+H]⁺.

### Preparation of Example 18 ((S)-(5-fluoro-1-((R)-5-(pyridin-2-yl)-2,3-dihydrobenzofuran-2-carbonyl)indolin-6-yl)(imino)(2-methoxyethyl)-λ⁶-sulfanone)

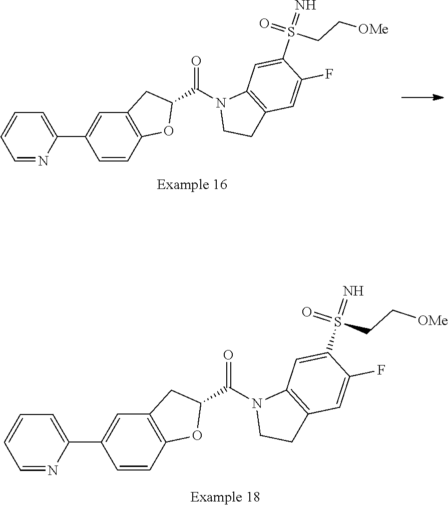 [SMILES: COCCS(=N)(=O)c1cc2c(cc1F)CCN2C(=O)[C@H]1Cc2cc(-c3ccccn3)ccc2O1 ; COCC[S@](=N)(=O)c1cc2c(cc1F)CCN2C(=O)[C@H]1Cc2cc(-c3ccccn3)ccc2O1]

Example 18 was obtained as the second eluent from chiral SFC purification of Example 16 using DAICEL CHIRALCEL OJ (250 mm×30 mm, 10 um) column; 50% (0.1% NH₃ in methanol) as co-solvent. ¹H NMR (400 MHz, DMSO-d6): δ 8.60 (br d, J=4.3 Hz, 1H), 8.52 (d, J=6.6 Hz, 1H), 8.01 (s, 1H), 7.93-7.78 (m, 3H), 7.38 (br d, J=9.9 Hz, 1H), 7.32-7.21 (m, 1H), 6.94 (d, J=8.4 Hz, 1H), 5.75 (t, J=7.9 Hz, 1H), 4.68 (s, 1H), 4.47-4.27 (m, 2H), 3.64-3.52 (m, 4H), 3.51-3.41 (m, 2H), 3.33-3.27 (m, 2H), 3.06 (s, 3H) ppm. LC/MS: 482.0 [M+H]⁺.

### Preparation of Example 19 (cyclopropyl(5-fluoro-1-((R)-5-(pyridin-2-yl)-2,3-dihydro-1H-indene-2-carbonyl)indolin-6-yl)(imino)-λ⁶-sulfanone)

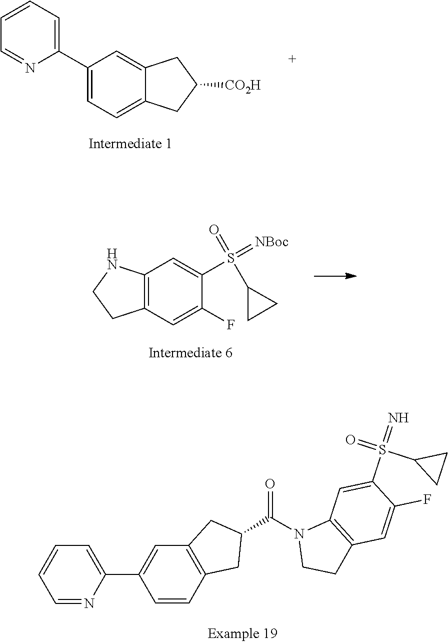

Example 19 was prepared using General Procedure A, employing Intermediate 1 and Intermediate 6 as starting materials. ¹H NMR (400 MHz, DMSO-d₆): δ 8.65-8.62 (m, 1H), 8.56 (d, J=6.6 Hz, 1H), 7.96-7.83 (m, 4H), 7.37-7.30 (m, 3H), 4.53 (s, 1H), 4.33 (br t, J=8.5 Hz, 2H), 3.77-3.69 (m, 1H), 3.28 (br d, J=6.2 Hz, 6H), 2.82-2.75 (m, 1H), 1.02-0.88 (m, 4H) ppm. LC/MS: 462.1 [M+H]⁺.

### Preparation of Example 20 ((R)-cyclopropyl(5-fluoro-1-((R)-5-(pyridin-2-yl)-2,3-dihydro-1H-indene-2-carbonyl)indolin-6-yl)(imino)-λ⁶-sulfanone)

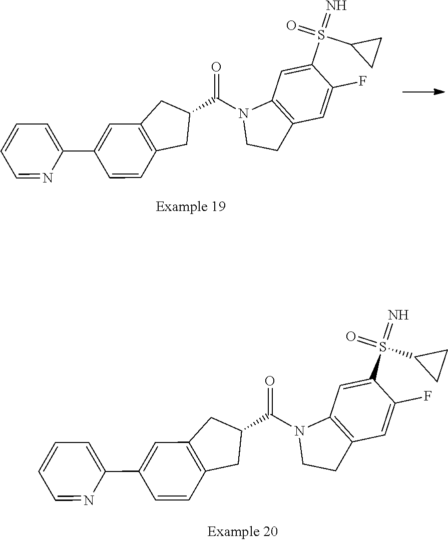 [SMILES: N=S(=O)(c1cc2c(cc1F)CCN2C(=O)[C@@H]1Cc2ccc(-c3ccccn3)cc2C1)C1CC1 ; N=[S@](=O)(c1cc2c(cc1F)CCN2C(=O)[C@@H]1Cc2ccc(-c3ccccn3)cc2C1)C1CC1]

Example 20 was obtained as the first eluent from chiral SFC purification of Example 19 using ChiralPak IH (250×30 mm×10 μm); 50% co-solvent (0.1% NH₃ in methanol). ¹H NMR (400 MHz, DMSO-d₆): δ 8.64 (d, J=4.3 Hz, 1H), 8.56 (d, J=6.5 Hz, 1H), 7.95-7.84 (m, 4H), 7.37-7.30 (m, 3H), 4.53 (s, 1H), 4.33 (br t, J=8.4 Hz, 2H), 3.73 (br t, J=8.0 Hz, 1H), 3.31-3.21 (m, 6H), 2.79 (br t, J=4.3 Hz, 1H), 1.06-0.90 (m, 4H) ppm. LC/MS: 462.1 [M+H]⁺.

### Preparation of Example 21 ((S)-cyclopropyl(5-fluoro-1-((R)-5-(pyridin-2-yl)-2,3-dihydro-1H-indene-2-carbonyl)indolin-6-yl)(imino)-λ⁶-sulfanone)

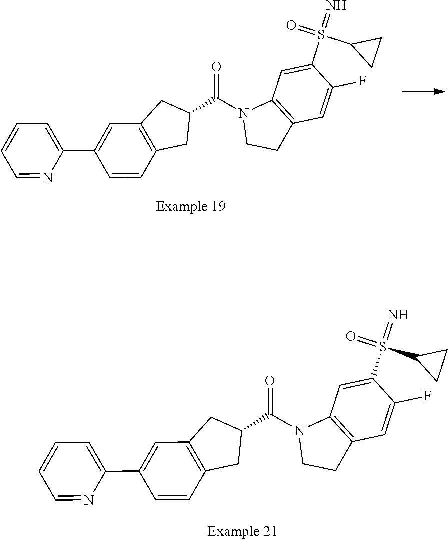 [SMILES: N=S(=O)(c1cc2c(cc1F)CCN2C(=O)[C@@H]1Cc2ccc(-c3ccccn3)cc2C1)C1CC1 ; N=[S@@](=O)(c1cc2c(cc1F)CCN2C(=O)[C@@H]1Cc2ccc(-c3ccccn3)cc2C1)C1CC1]

Example 21 was obtained as the second eluent from chiral SFC purification of Example 19 using ChiralPak IH (250×30 mm×10 μm); 50% co-solvent (0.1% NH₃ in methanol). ¹H NMR (400 MHz, DMSO-d₆): δ 8.64 (d, J=4.4 Hz, 1H), 8.56 (d, J=6.5 Hz, 1H), 7.96-7.83 (m, 4H), 7.38-7.30 (m, 3H), 4.53 (s, 1H), 4.33 (br t, J=8.4 Hz, 2H), 3.73 (quin, J=8.0 Hz, 1H), 3.31-3.20 (m, 6H), 2.78 (br d, J=4.9 Hz, 1H), 1.06-0.90 (m, 4H) ppm. LC/MS: 462.1 [M+H]⁺.

### Preparation of Example 22 ((5-fluoro-1-((R)-5-(pyridin-2-yl)-2,3-dihydro-1H-indene-2-carbonyl)indolin-6-yl)(methyl)(methylimino)-l6-sulfanone)

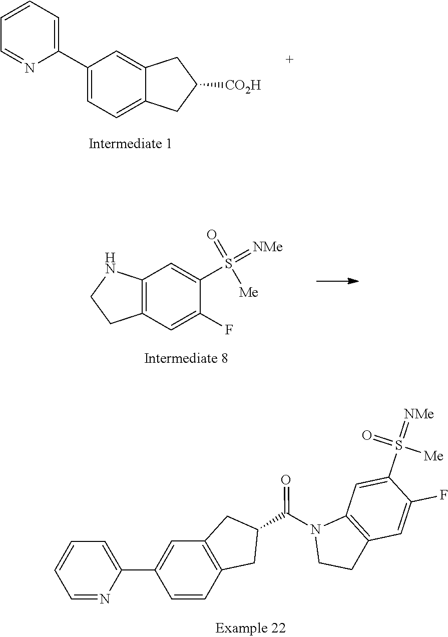

Example 22 was prepared using General Procedure A (Step One only), employing Intermediate 1 and Intermediate 8 as starting materials. ¹H NMR (400 MHz, DMSO-d₆): δ 8.64 (br d, J=4.0 Hz, 1H), 8.55 (d, J=6.6 Hz, 1H), 7.95-7.83 (m, 4H), 7.40-7.30 (m, 3H), 4.34 (br t, J=8.5 Hz, 2H), 3.73 (quin, J=8.0 Hz, 1H), 3.33-3.26 (m, 6H), 3.19 (s, 3H), 2.47 (s, 3H) ppm. LC/MS: 450.1 [M+H]⁺.

### Preparation of Example 23 ((R)-(5-fluoro-1-((R)-5-(pyridin-2-yl)-2,3-dihydro-1H-indene-2-carbonyl)indolin-6-yl)(methyl)(methylimino)-λ⁶-sulfanone)

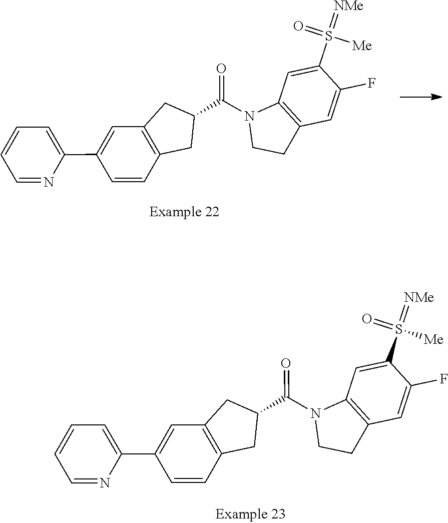

Example 23 was obtained as the first eluent from chiral SFC purification of Example 22 using ChiralPak IH (250×30 mm×10 μm); 50% co-solvent (0.1% NH₃ in methanol). ¹H NMR (400 MHz, DMSO-d₆): δ 8.64 (d, J=4.8 Hz, 1H), 8.54 (d, J=6.6 Hz, 1H), 7.96-7.83 (m, 4H), 7.41-7.30 (m, 3H), 4.34 (t, J=8.5 Hz, 2H), 3.73 (quin, J=8.0 Hz, 1H), 3.32-3.23 (m, 6H), 3.19 (s, 3H), 2.46 (s, 3H) ppm. LC/MS: 450.1 [M+H]⁺.

### Preparation of Example 24 ((S)-(5-fluoro-1-((R)-5-(pyridin-2-yl)-2,3-dihydro-1H-indene-2-carbonyl)indolin-6-yl)(methyl)(methylimino)-λ⁶-sulfanone)

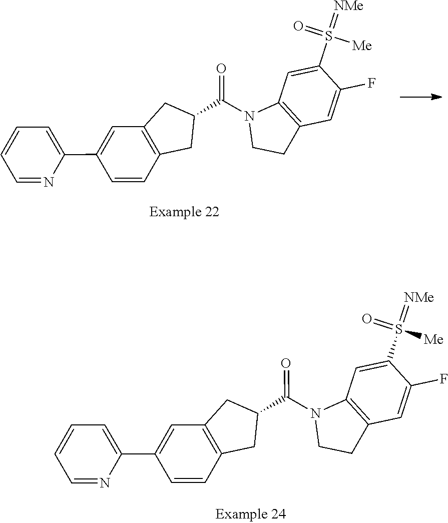

Example 24 was obtained as the second eluent from chiral SFC purification of Example 22 using ChiralPak IH (250×30 mm×10 μm); 50% co-solvent (0.1% NH₃ in methanol). ¹H NMR (400 MHz, DMSO-d₆): δ 8.64 (br d, J=4.5 Hz, 1H), 8.54 (d, J=6.5 Hz, 1H), 7.96-7.83 (m, 4H), 7.41-7.30 (m, 3H), 4.34 (br t, J=8.4 Hz, 2H), 3.78-3.69 (m, 1H), 3.28 (br d, J=8.0 Hz, 6H), 3.18 (s, 3H), 2.46 (s, 3H) ppm. LC/MS: 450.1 [M+H]⁺.

### Preparation of Example 25 (ethyl(5-fluoro-2-methyl-1-((R)-5-(pyridin-2-yl)-2,3-dihydro-1H-indene-2-carbonyl)indolin-6-yl)(imino)-λ⁶-sulfanone)

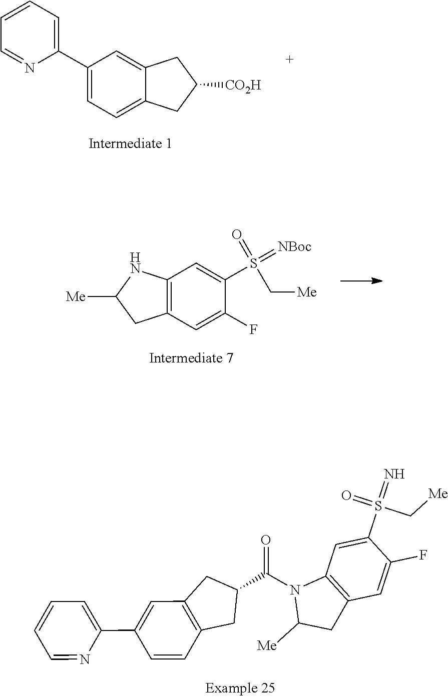

Example 25 was prepared using General Procedure A, employing Intermediate 1 and Intermediate 7 as starting material. ¹H NMR (400 MHz, DMSO-d₆): δ 8.64 (br s, 1H), 8.60-8.52 (m, 1H), 7.99-7.83 (m, 4H), 7.43-7.26 (m, 3H), 4.96-4.83 (m, 1H), 4.58 (d, J=17.5 Hz, 1H), 3.75 (quin, J=8.3 Hz, 1H), 3.52 (br dd, J=8.9, 16.8 Hz, 1H), 3.47-3.35 (m, 2H), 3.30-3.18 (m, 3H), 3.17-3.06 (m, 1H), 2.81 (br d, J=16.9 Hz, 1H), 1.29 (br d, J=6.1 Hz, 3H), 1.10 (br t, J=7.2 Hz, 3H) ppm. LC/MS: 464.2 [M+H]⁺.

### Preparation of Example 26 ((R)-ethyl((R)-5-fluoro-2-methyl-1-((R)-5-(pyridin-2-yl)-2,3-dihydro-1H-indene-2-carbonyl)indolin-6-yl)(imino)-λ⁶-sulfanone)

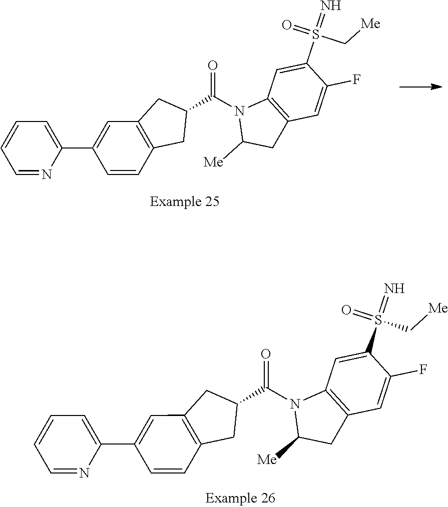 [SMILES: CCS(=N)(=O)c1cc2c(cc1F)CC(C)N2C(=O)[C@@H]1Cc2ccc(-c3ccccn3)cc2C1 ; CC[S@@](=N)(=O)c1cc2c(cc1F)C[C@@H](C)N2C(=O)[C@@H]1Cc2ccc(-c3ccccn3)cc2C1]

Example 26 was obtained as the first eluent from chiral SFC purification of Example 25 using ChiralPak IH (250×30 mm×10 μm); 50% co-solvent (0.1% NH₃ in methanol). ¹H NMR (400 MHz, DMSO-d6): δ 8.64 (d, J=4.0 Hz, 1H), 8.55 (d, J=6.6 Hz, 1H), 8.01-7.81 (m, 4H), 7.39 (d, J=9.7 Hz, 1H), 7.35-7.27 (m, 2H), 4.96-4.82 (m, 1H), 4.60 (s, 1H), 3.75 (quin, J=8.1 Hz, 1H), 3.52 (br dd, J=8.7, 16.9 Hz, 1H), 3.46-3.36 (m, 2H), 3.31-3.17 (m, 3H), 3.11 (br dd, J=7.7, 16.0 Hz, 1H), 2.81 (br d, J=16.7 Hz, 1H), 1.29 (d, J=6.2 Hz, 3H), 1.10 (t, J=7.3 Hz, 3H) ppm. LC/MS: 464.2 [M+H]⁺.

### Preparation of Example 27 ((S)-ethyl((R)-5-fluoro-2-methyl-1-((R)-5-(pyridin-2-yl)-2,3-dihydro-1H-indene-2-carbonyl)indolin-6-yl)(imino)-λ⁶-sulfanone)

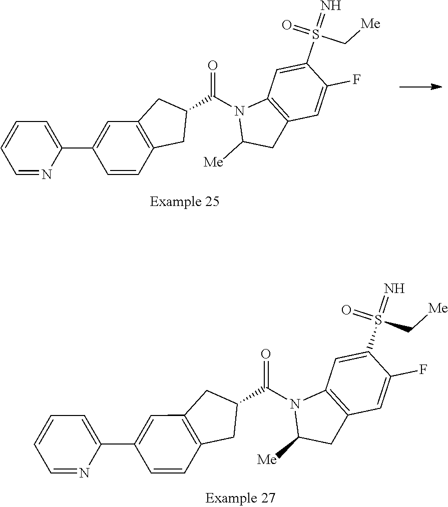 [SMILES: CCS(=N)(=O)c1cc2c(cc1F)CC(C)N2C(=O)[C@@H]1Cc2ccc(-c3ccccn3)cc2C1 ; CC[S@](=N)(=O)c1cc2c(cc1F)C[C@@H](C)N2C(=O)[C@@H]1Cc2ccc(-c3ccccn3)cc2C1]

Example 27 was obtained as the second eluent from chiral SFC purification of Example 25 using ChiralPak IH (250×30 mm×10 μm); 50% co-solvent (0.1% NH₃ in methanol). ¹H NMR (400 MHz, DMSO-d6): δ 8.64 (d, J=4.0 Hz, 1H), 8.56 (d, J=6.6 Hz, 1H), 8.02-7.78 (m, 4H), 7.39 (d, J=9.8 Hz, 1H), 7.36-7.28 (m, 2H), 4.96-4.80 (m, 1H), 4.56 (s, 1H), 3.81-3.69 (m, 1H), 3.52 (br dd, J=9.0, 16.7 Hz, 1H), 3.46-3.36 (m, 2H), 3.29-3.18 (m, 3H), 3.11 (br dd, J=7.4, 15.8 Hz, 1H), 2.81 (br d, J=17.0 Hz, 1H), 1.29 (d, J=6.2 Hz, 3H), 1.10 (t, J=7.3 Hz, 3H) ppm. LC/MS: 464.2 [M+H]⁺.

### Preparation of Example 28 ((S)-ethyl((S)-5-fluoro-2-methyl-1-((R)-5-(pyridin-2-yl)-2,3-dihydro-1H-indene-2-carbonyl)indolin-6-yl)(imino)-λ⁶-sulfanone)

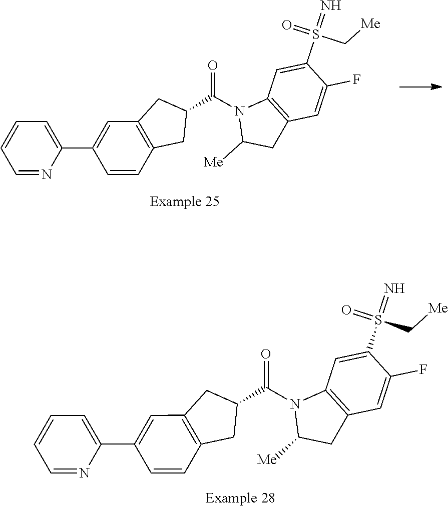 [SMILES: CCS(=N)(=O)c1cc2c(cc1F)CC(C)N2C(=O)[C@@H]1Cc2ccc(-c3ccccn3)cc2C1 ; CC[S@](=N)(=O)c1cc2c(cc1F)C[C@H](C)N2C(=O)[C@@H]1Cc2ccc(-c3ccccn3)cc2C1]

Example 28 was obtained as the third eluent from chiral SFC purification of Example 25 using ChiralPak IH (250×30 mm×10 μm); 50% co-solvent (0.1% NH₃ in methanol). ¹H NMR (400 MHz, DMSO-d6): δ 8.63 (d, J=4.0 Hz, 1H), 8.56 (d, J=6.6 Hz, 1H), 7.96-7.81 (m, 4H), 7.44-7.28 (m, 3H), 4.95-4.83 (m, 1H), 4.57 (s, 1H), 3.81-3.70 (m, 1H), 3.58-3.34 (m, 3H), 3.28-3.08 (m, 4H), 2.81 (br d, J=17.0 Hz, 1H), 1.29 (br d, J=6.1 Hz, 3H), 1.11 (t, J=7.3 Hz, 3H) ppm. LC/MS: 464.2 [M+H]⁺.

### Preparation of Example 29 ((R)-ethyl((S)-5-fluoro-2-methyl-1-((R)-5-(pyridin-2-yl)-2,3-dihydro-1H-indene-2-carbonyl)indolin-6-yl)(imino)-λ⁶-sulfanone)

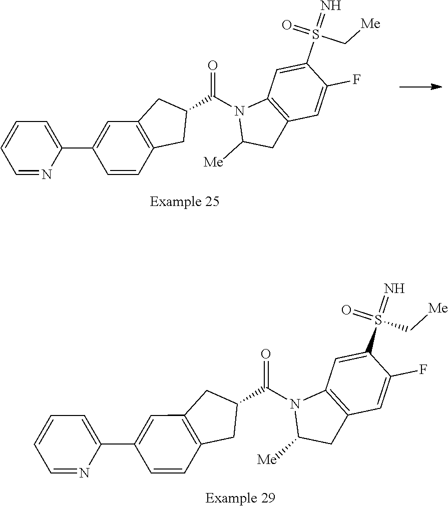 [SMILES: CCS(=N)(=O)c1cc2c(cc1F)CC(C)N2C(=O)[C@@H]1Cc2ccc(-c3ccccn3)cc2C1 ; CC[S@@](=N)(=O)c1cc2c(cc1F)C[C@H](C)N2C(=O)[C@@H]1Cc2ccc(-c3ccccn3)cc2C1]

Example 29 was obtained as the fourth eluent from chiral SFC purification of Example 25 using ChiralPak IH (250×30 mm×10 μm); 50% co-solvent (0.1% NH₃ in methanol). ¹H NMR (400 MHz, DMSO-d6): δ 8.64 (br d, J=4.5 Hz, 1H), 8.57 (br d, J=6.6 Hz, 1H), 7.98-7.78 (m, 4H), 7.44-7.27 (m, 3H), 4.95-4.82 (m, 1H), 4.53 (s, 1H), 3.81-3.69 (m, 1H), 3.58-3.34 (m, 3H), 3.27-3.09 (m, 4H), 2.81 (br d, J=17.1 Hz, 1H), 1.29 (br d, J=6.0 Hz, 3H), 1.10 (br t, J=7.3 Hz, 3H) ppm. LC/MS: 464.2 [M+H]⁺.

### Preparation of Example 30 ((R)—N-ethyl-5-fluoro-1-(5-(pyridin-2-yl)-2,3-dihydro-1H-indene-2-carbonyl)indoline-6-sulfonamide)

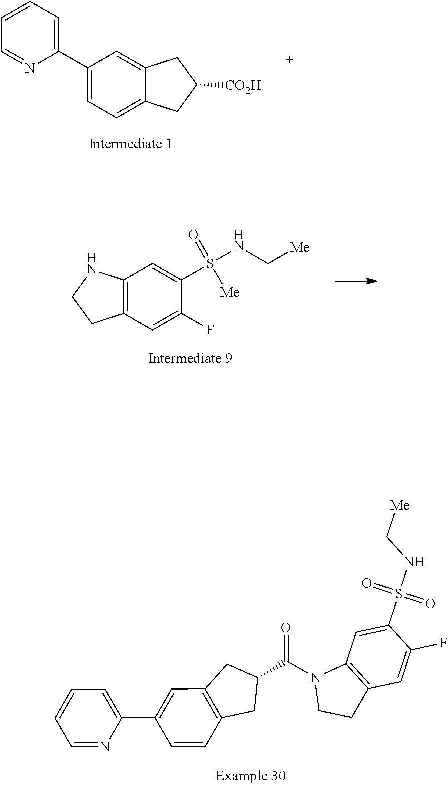

Example 30 was prepared using General Procedure A (Step One only), employing Intermediate 1 and Intermediate 9 as starting materials. ¹H NMR (400 MHz, DMSO-d₆): δ 8.64 (dd, J=0.8, 4.8 Hz, 1H), 8.53 (d, J=6.6 Hz, 1H), 7.96-7.83 (m, 4H), 7.77 (br t, J=5.4 Hz, 1H), 7.40-7.29 (m, 3H), 4.34 (t, J=8.5 Hz, 2H), 3.78-3.68 (m, 1H), 3.32-3.20 (m, 6H), 2.93-2.80 (m, 2H), 0.98 (t, J=7.2 Hz, 3H) ppm. LC/MS: 466.0 [M+H]⁺.

### Preparation of Example 31 ((R)—N-ethyl-5-fluoro-1-(5-(pyridin-2-yl)-2,3-dihydrobenzofuran-2-carbonyl)indoline-6-sulfonamide)

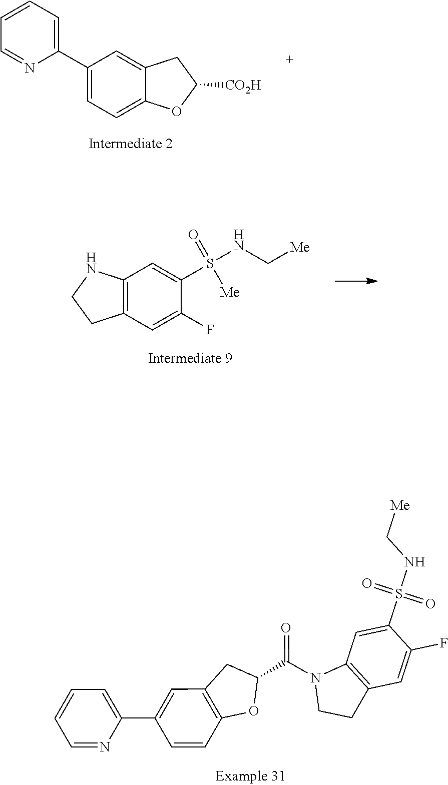

Example 31 was prepared using General Procedure A (Step One only), employing Intermediate 2 and Intermediate 9 as starting materials. ¹H NMR (400 MHz, DMSO-d6): δ 8.60 (br d, J=4.4 Hz, 1H), 8.48 (d, J=6.6 Hz, 1H), 8.00 (s, 1H), 7.92-7.80 (m, 4H), 7.41 (br d, J=9.8 Hz, 1H), 7.30-7.24 (m, 1H), 6.94 (d, J=8.3 Hz, 1H), 5.74 (br dd, J=7.1, 9.0 Hz, 1H), 4.46-4.26 (m, 2H), 3.67-3.53 (m, 2H), 3.31 (br s, 2H), 2.86 (quin, J=6.7 Hz, 2H), 0.97 (t, J=7.2 Hz, 3H) ppm. LC/MS: 468.0 [M+H]⁺.

### Preparation of Example 32 (N—(((R)-5-fluoro-2-methyl-1-((R)-5-(pyridin-2-yl)-2,3-dihydro-1H-indene-2-carbonyl)indolin-6-yl)sulfonyl)butyramide)

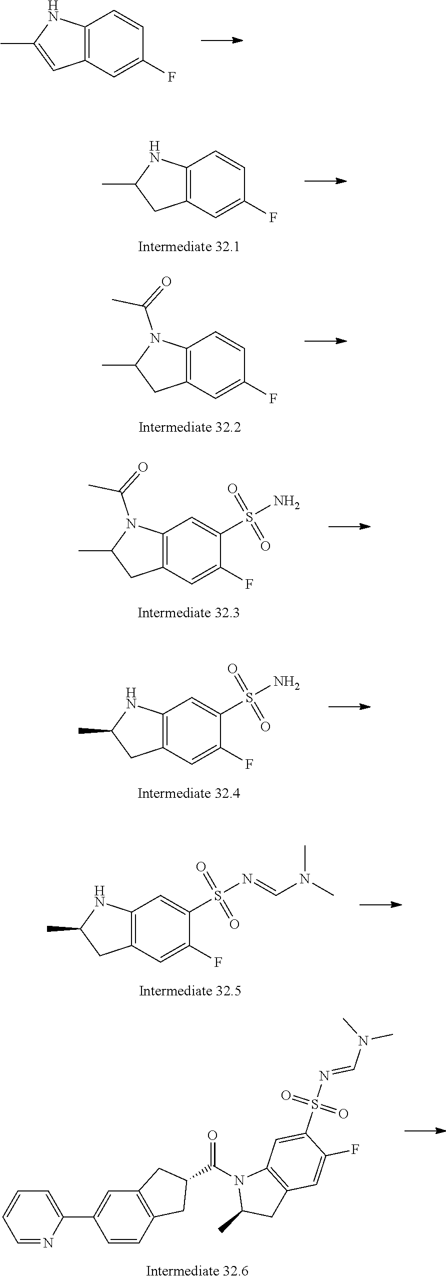

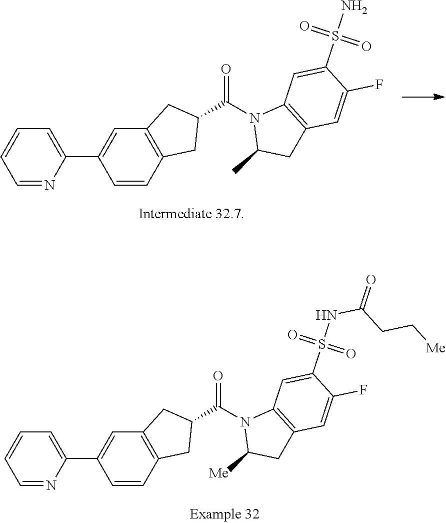 [SMILES: C[C@@H]1Cc2cc(F)c(S(N)(=O)=O)cc2N1C(=O)[C@@H]1Cc2ccc(-c3ccccn3)cc2C1 ; CCCC(=O)NS(=O)(=O)c1cc2c(cc1F)C[C@@H](C)N2C(=O)[C@@H]1Cc2ccc(-c3ccccn3)cc2C1]

Intermediate 32.7 was prepared according to the procedures described in PCT Application No. PCT/US2023/022679.

Preparation of Intermediate 32.1: 5-fluoro-2-methylindoline (40 g, 268 mmol, 1 eq) was taken up in acetic acid (200 mL, 5V). NaBH₃CN (50 g, 815 mmol, 3 eq) was added portion-wise while maintaining the temperature below 10° C. The resulting solution was warmed to RT and stirred for 3 h, at which time the reaction was diluted with ice-cold water (500 mL). The reaction was then extracted with EtOAc (3×100 mL). The combined organic layer was washed with brine (750 mL), dried over Na₂SO₄ and concentrated in vacuo. The crude Intermediate 32.1 was isolated as a thick yellow oil and used in the next step without further purification. LC/MS: 151.2 [M+H]⁺.

Preparation of Intermediate 32.2: To a stirred solution of Intermediate 32.1 (35 g, 86.6 mmol) in DCM (300 mL) was added triethylamine (35 mL, 1 vol) followed by acetyl chloride (35 mL, 1 vol) at 0° C. The reaction mixture was stirred at RT for 2 h. The residue was quenched with cold water (200 mL) and extracted with EtOAc (3×100 mL). The combined organic layer was washed with brine (750 mL), dried over Na₂SO₄ and concentrated in vacuo. The crude residue was purified by column chromatography (100-200 silica gel, eluted 8% EtOAc-Hexane) to afford Intermediate 32.2. LC/MS: 193.2 [M+H]⁺.

Preparation of Intermediate 32.3: To a stirred solution of Intermediate 32.2 (35 g, 0.011 mol) in chlorosulfonic acid (250 mL, 10 V) at 0° C. under nitrogen and the reaction mixture was stirred at 50° C. for 3 h. Upon completion of the reaction, the mixture was diluted with ice-cold water. The precipitate was filtered, taken up in dichloromethane (50 mL), and added to a solution of concentrated ammonium hydroxide (20 mL). After vigorous stirring for 15 minutes at RT, the solvent was removed under pressure and the resulting solid was filtered and rinsed with water to afford Intermediate 32.3. LC/MS: 272.3 [M+H]⁺.

Preparation of Intermediates 32.4: A racemic mixture of Intermediate 32.3 (30 g, 110 mmol, 1 equiv) in 500 mL of 2N sodium hydroxide was heated at 100° C. for 3 hours. The reaction was cooled to RT and the pH was adjusted to 7 with acetic acid. The precipitate was filtered, washed with water, and dried under vacuum. The resulting de-acetylated racemic mixture was then subjected to chiral SFC purification using a ChiralPak IG 240×4.6 mm column with a mobile phase consisting of 70:20:10 hexane:methanol:MTBE. Intermediate 32.4 was collected as the second eluent (retention time 13.1 min). LC/MS: 230.1 [M+H]⁺.

Intermediate 32.4 was taken up in THF (10 V) and dimethylformamide dimethyl acetal (1.2 equiv) was added at once. The solution was stirred at RT for 30 minutes and the solvent was then removed under reduced pressure to afford the crude residue Intermediate 32.5 (LC/MS: 286.1 [M+H]⁺) which was used directly in the next step without further purification.

Intermediate 32.5 was taken up in acetonitrile (10 V). Intermediate 1 (1 equiv) was added, followed by TCFH (2 equiv). The suspension was placed in a water bath at rt and N-methylimidazole (5 equiv) was added dropwise. The solution was stirred at RT for 1 hour, at which time LC/MS analysis indicated complete conversion to Intermediate 32.6 (LC/MS: 507.2 [M+H]). Hydrazine hydrate (50 equiv) was then added all at once and the solution was allowed to stir at RT for 30 minutes, after which time water was added (5 V). The resulting precipitate was filtered and dried under reduced pressure to afford Intermediate 32.7. ¹H NMR (400 MHz, DMSO-d6): δ 8.69 (d, J=4.9 Hz, 1H), 8.54 (d, J=6.6 Hz, 1H), 8.10-7.84 (m, 4H), 7.58 (s, 2H), 7.51-7.24 (m, 3H), 4.90 (t, J=7.5 Hz, 1H), 3.77 (p, J=8.2 Hz, 1H), 3.46 (ddt, J=34.0, 17.5, 8.7 Hz, 3H), 3.20 (ddd, J=59.1, 16.3, 8.3 Hz, 2H), 2.80 (d, J=16.9 Hz, 1H), 1.30 (d, J=6.2 Hz, 3H) ppm. LC/MS: 452.1 [M+H]⁺.

Intermediate 32.7 (250 mg, 0.55 mmol) was taken up in THF (5 mL), and triethylamine (112 mg, 2 eq), DMAP (14 mg, 0.2 eq), and n-butyric anhydride (88 mg, 1.0 eq) were added at RT. After 1 hour, silica gel (5 grams) was added, and the solvent was removed under reduced pressure. The resulting free flowing solid was purified by flash column chromatography (0 to 20% methanol in DCM) to afford Example 32. ¹H NMR (400 MHz, DMSO): δ 12.38 (s, 1H), 8.82-8.49 (m, 2H), 7.98 (s, 1H), 7.97-7.83 (m, 3H), 7.43 (d, J=9.9 Hz, 1H), 7.37-7.28 (m, 2H), 4.90 (p, J=6.7 Hz, 1H), 3.76 (p, J=8.3 Hz, 1H), 3.66-3.48 (m, 1H), 3.42 (ddd, J=16.1, 8.3, 3.1 Hz, 2H), 3.27 (dd, J=15.9, 8.6 Hz, 1H), 3.14 (dd, J=16.1, 7.8 Hz, 1H), 2.83 (d, J=17.1 Hz, 1H), 2.22 (t, J=7.2 Hz, 2H), 2.08 (s, 1H), 1.44 (h, J=7.3 Hz, 2H), 1.30 (d, J=6.3 Hz, 3H), 0.78 (t, J=7.4 Hz, 3H) ppm. LC/MS: 522.2 [M+H]⁺.

### Biological Assays

### HSV-2 Cellomics Assay

Compounds were tested for their ability to inhibit HSV-2 replication by monitoring the expression of the HSV protein gD using a high-throughput immunofluorescence-based assay. 10-dose, 3-fold serial dilutions of compounds were prepared at starting concentrations of 0.2 or 2 mM in 100% DMSO. 250 nl of compounds were spotted in quadruplicates onto black collagen-coated 384-well microplates with clear bottom (Greiner cat #781956) using a Labcyte ECHO acoustic dispenser. The final starting concentration in the assay was either 1 or 10 M. DMSO (no compound) and pritelivir were included on each microplate as negative and positive controls, respectively.

ARPE-19 cells (ATCC cat #CRL-2302) were maintained DMEM/F-12 Glutamax medium (Thermo Fisher Scientific cat #10565018) supplemented with 10% FBS (Corning cat #35-011-CV) and 1% Penicillin-Streptomycin (cat #30-002-CI). Prior to confluence, cells were transferred to a centrifuge tube and spun for 5 minutes at 1000 rpm. Cells were resuspended in assay medium (DMEM/F-12 Glutamax, 2% FBS, 1% Penicillin-Streptomycin) and counted. Cell density was adjusted to 150,000 cells/ml and infected with HSV-2 virus (MS strain, ATCC cat #VR-540) at a MOI of 0.06 in a 50 ml conical tube for 1 hour under constant rocking. Cells were then spun for 5 minutes at 1000 rpm and the media replaced with assay medium containing either 2% FBS or 10% human serum (EMD Millipore cat #S1-100ML). 50 μl of the infection suspension were added into microplate wells that were pre-spotted with compounds (7,500 cells per well). Plates were incubated for 16 hours at 37° C.

Cell culture medium was aspirated using a Biomek Fx and 50 μl of a paraformaldehyde solution (Electron Microscopy Sciences cat #15712-5) diluted to 4% in DPBS (Corning cat #21-031-CM) were added per well. After a 30-minute incubation at room temperature, plates were washed 4 times with 100 μl/well of PBS using a Biotek plate washer. A 1:500 solution of primary antibody (anti-HSV gD, Virusys cat #P1103) was prepared in permeabilization buffer (Invitrogen cat #00-8333-56) and 50 μl were added into wells. After a 1-hour incubation at room temperature, 50 μl of a 1:1000 solution of secondary antibody (Alexa Fluor 488 goat anti-mouse, Thermo Fisher Scientific cat #A11001) and DAPI (Thermo Fisher Scientific cat #62248) in permeabilization buffer were added into wells and plates were incubated in the dark at room temperature for 1 hour. Plates were washed 4 times again and 50 μl of DPBS were added to all wells prior to sealing the plates with a black adhesive seal. Fluorescence was measured on a Cellomics plate reader.

Data analysis was carried out using the Thermo Scientific HCS Studio software. Briefly, cells were identified using the DAPI nuclear stain and thresholds were set to filter cells out based on shape and size. A second threshold based on green fluorescence intensity (detection of HSV-2 gD) was set to identify HSV-2 infected cells. Data was reported as the average fluorescence intensity of HSV-2 infected cells. EC50 values were defined as the compound concentration that caused a 50% decrease in the average fluorescence intensity and was calculated using a sigmoidal dose-response model to generate curve fits. EC50 for pritelivir in this assay was 150 nM. Data for certain compounds are reported in Table 1 below.

TABLE 1

| Ex- | EC₅₀-HSV2- | EC₅₀-HSV2-10% |
|---|---|---|
| ample | FBS (nM) | Human Serum (nM) |
| 2 | 18 | 71 |
| 3 | 73 | 258 |
| 5 | 178 | 474 |
| 6 | 40 | 81 |
| 8 | 16 | 77 |
| 9 | 52 | 218 |
| 11 | 124 | 406 |
| 12 | 35 | 98 |
| 13 | 31 | 55 |
| 14 | 25 | 54 |
| 15 | 42 | 87 |
| 17 | 39 | 59 |
| 18 | 92 | 208 |
| 19 | 56 | 453 |
| 20 | 33 | 248 |
| 21 | 98 | 949 |
| 22 | 211 | 737 |
| 23 | 155 | 522 |
| 25 | 72 | 203 |
| 26 | 30 | 92 |
| 27 | 55 | 113 |
| 30 | 5 | 304 |
| 31 | 10 | 570 |
| 32 | 231 | 2376 |

Unless otherwise defined, all technical and scientific terms used herein have the same meaning as commonly understood by one of ordinary skill in the art to which this disclosure belongs.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A compound of Formula (I) 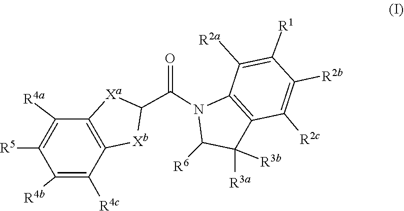
or a pharmaceutically acceptable salt thereof, wherein
R¹ is 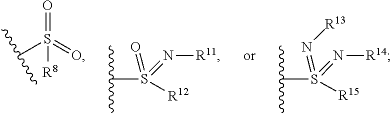
R^{2a}, R^{2b}, and R^{2c} are each independently H, halogen, C₁₋₆ alkyl, C₁₋₆ haloalkyl, C₁₋₆ alkoxy, —CN, C₁₋₆haloalkoxy, or —SCF₃;
R^{3a} and R^{3b} are each independently H, halogen, C₁₋₆ alkyl, or C₁₋₆ haloalkyl;
X^{a} and X^{b} are each independently O or CR^{7a}R^{7b};
each R^{7a} is independently H, C₁₋₆ alkyl, or C₁₋₆ haloalkyl;
each R^{7b} is independently H, C₁₋₆ alkyl, or C₁₋₆ haloalkyl;
R^{4a}, R^{4b}, and R^{4c} are each independently H, halogen, C₁₋₆ alkyl, C₁₋₆ haloalkyl, C₁₋₆ alkoxy, —CN, —SCF₃, or C₃₋₆ cycloalkyl optionally substituted with Z⁴;
R⁵ is C₆₋₁₀ aryl or heteroaryl; wherein the aryl or heteroaryl of R⁵ is optionally substituted with one to three Z⁵, which may be the same or different; the heteroaryl of R⁵ is 5 to 10 membered heteroaryl having one to three heteroatoms each independently N, O, or S;
R⁶ is H, C₁₋₆ alkyl, C₁₋₆ haloalkyl, C₂₋₈ alkoxyalkyl, or C₃₋₆ cycloalkyl, wherein the cycloalkyl of R⁶ is optionally substituted with one to three Z⁶;
R⁸ is NHR⁹ or NR⁹R¹⁰;
each R⁹, R¹⁰, and R¹² is independently C₁₋₆ alkyl, C₁₋₆ haloalkyl, —COR^{9a}, or C₃₋₆ cycloalkyl; the alkyl or cycloalkyl of R⁹, R¹⁰, or R¹² is optionally substituted with one to three groups independently selected from —OR^{12a}, and —SR^{12a};
R¹¹ is H, C₁₋₆ alkyl, C₁₋₆ haloalkyl, —COR^{9a}, or C₃₋₆ cycloalkyl; the alkyl or cycloalkyl of R¹¹ is optionally substituted with one to three groups each independently —OR^{12a} or —SR^{12a};
each R¹³ and R¹⁴ is independently H or C₁₋₆ alkyl;
R¹⁵ is C₁₋₆ alkyl, C₁₋₆ haloalkyl, or C₃₋₆ cycloalkyl; the alkyl or cycloalkyl of R¹⁵ is optionally substituted with one to three groups each independently —OR^{12a} or —SR^{12a};
each R^{9a} and R^{12a} is independently C₁₋₆ alkyl; and
each Z⁴, Z⁵, and Z⁶ is independently halogen, —CN, C₁₋₆ alkyl, C₁₋₆ haloalkyl, C₁₋₆ alkoxy, or C₁₋₆haloalkoxy;
provided that when R¹¹ is H, R¹² is not —CH₃.

2. The compound of claim 1, or a pharmaceutically acceptable salt thereof, wherein the compound of Formula (I) is represented by Formula (Ia)

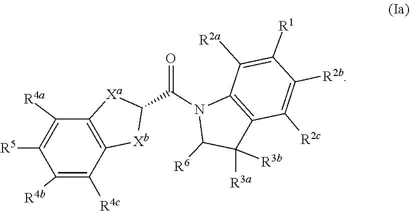

3. The compound of claim 1, or a pharmaceutically acceptable salt thereof, wherein the compound of Formula (I) is represented by Formula (II)

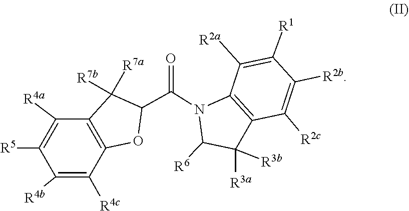

4. The compound of claim 1, or a pharmaceutically acceptable salt thereof, wherein the compound of Formula (I) is represented by Formula (IIa)

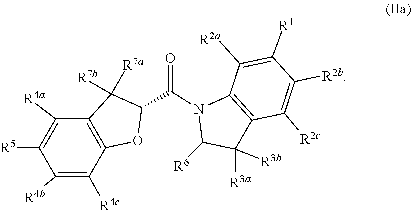

5. The compound of claim 1, or a pharmaceutically acceptable salt thereof, wherein the compound of Formula (I) is represented by Formula (III)

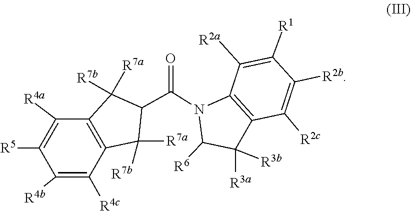

6. The compound of claim 1, or a pharmaceutically acceptable salt thereof, wherein the compound of Formula (I) is represented by Formula (IIIa)

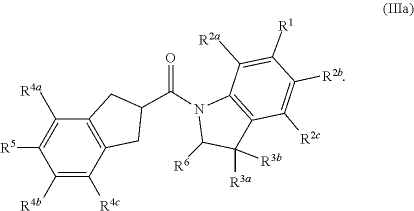

7. The compound of claim 1, or a pharmaceutically acceptable salt thereof, wherein the compound of Formula (I) is represented by Formula (IIIb)

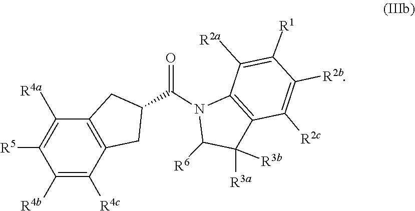

8. The compound of claim 1, or a pharmaceutically acceptable salt thereof, wherein the compound of Formula (I) is represented by Formula (IIIc)

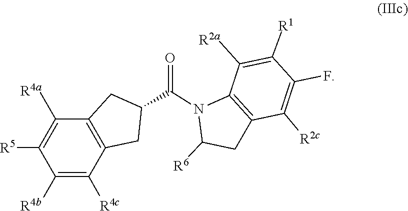

9. The compound of claim 1, or a pharmaceutically acceptable salt thereof, wherein the compound of Formula (I) is represented by Formula (IIId)

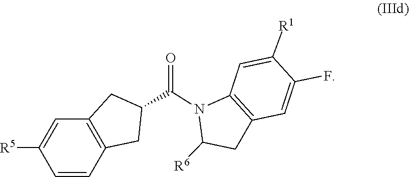

10. The compound of claim 1, or a pharmaceutically acceptable salt thereof, wherein the compound of Formula (I) is represented by Formula (IIIe)

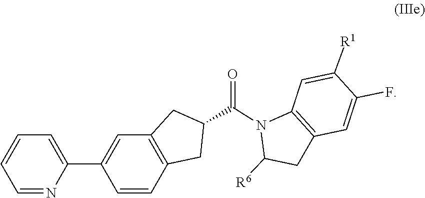

11. The compound of any one of claims 1-8, or a pharmaceutically acceptable salt thereof, wherein R^{2a} is H.

12. The compound of any one of claims 1-8 and 11, or a pharmaceutically acceptable salt thereof, wherein R^{2b} is halogen.

13. The compound of any one of claims 1-8 and 11, or a pharmaceutically acceptable salt thereof, wherein R^{2b} is F.

14. The compound of any one of claims 1-8 and 11-13, or a pharmaceutically acceptable salt thereof, wherein R^{2c} is H.

15. The compound of any one of claims 1-7 and 11-14, or a pharmaceutically acceptable salt thereof, wherein R^{3a} is H.

16. The compound of any one of claims 1-7 and 11-15, or a pharmaceutically acceptable salt thereof, wherein R^{3b} is H.

17. The compound of any one of claims 1-8 and 11-16, or a pharmaceutically acceptable salt thereof, wherein R^{4a} is H.

18. The compound of any one of claims 1-8 and 11-17, or a pharmaceutically acceptable salt thereof, wherein R^{4b} is H.

19. The compound of any one of claims 1-8 and 11-18, or a pharmaceutically acceptable salt thereof, wherein R^{4c} is H.

20. The compound of any one of claims 1-9 and 11-22, or a pharmaceutically acceptable salt thereof, wherein R⁵ is heteroaryl; wherein the heteroaryl of R⁵ may be optionally substituted with one to three Z⁵, which may be the same or different.

21. The compound of any one of claims 1-9 and 11-20, or a pharmaceutically acceptable salt thereof, wherein R⁵ is pyridyl optionally substituted with one to three Z⁵, which may be the same or different.

22. The compound of any one of claims 1-9 and 11-21, or a pharmaceutically acceptable salt thereof, wherein R⁵ is pyridyl.

23. The compound of any one of claims 1-22, or a pharmaceutically acceptable salt thereof, wherein R⁶ is H, C₁₋₆ alkyl or C₁₋₆ haloalkyl.

24. The compound of any one of claims 1-23, or a pharmaceutically acceptable salt thereof, wherein R⁶ is H.

25. The compound of any one of claims 1-23, or a pharmaceutically acceptable salt thereof, wherein R⁶ is —CH₃.

26. The compound of any one of claims 1-25, or a pharmaceutically acceptable salt thereof, wherein R¹ is

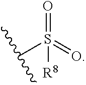

27. The compound of any one of claims 1-25, or a pharmaceutically acceptable salt thereof, wherein
R¹ is 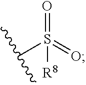
R⁸ is NHR⁹ or NR⁹R¹⁰;
each R⁹ and R¹⁰ is independently C₁₋₆ alkyl, C₁₋₆ haloalkyl, —COR^{9a}, or C₃₋₆ cycloalkyl;
the C₁₋₆ alkyl of R⁹ or R¹⁰ is optionally substituted with —OR^{12a}; and
each R^{9a} and R^{12a} is independently C₁₋₆ alkyl.

28. The compound of any one of claims 1-25, or a pharmaceutically acceptable salt thereof, wherein
R¹ is 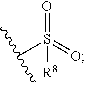
R⁸ is NHR⁹;
R⁹ is C₁₋₆ alkyl, or —COR^{9a}; and
R^{9a} is C₁₋₆ alkyl.

29. The compound of any one of claims 1-28, or a pharmaceutically acceptable salt thereof, wherein R¹ is

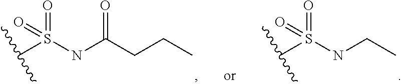

30. The compound of any one of claims 1-25, or a pharmaceutically acceptable salt thereof, wherein R¹ is

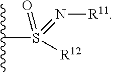

31. The compound of any one of claims 1-25, or a pharmaceutically acceptable salt thereof, wherein
R¹ is 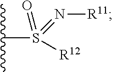
R¹¹ is H;
R¹² is —CH₂OR^{12a}, C₂₋₆ alkyl, C₁₋₆ haloalkyl, or C₃₋₆ cycloalkyl; the alkyl or cycloalkyl of R¹² is optionally substituted with —OR^{12a}; and
R^{12a} is C₁₋₆ alkyl.

32. The compound of any one of claims 1-25, or a pharmaceutically acceptable salt thereof, wherein
R¹ is 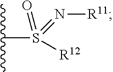
R¹¹ is H;
R¹² is C₂₋₆ alkyl or C₃₋₆ cycloalkyl; the alkyl or cycloalkyl or R¹² is optionally substituted with —OR^{12a}; and
R^{12a} is C₁₋₆ alkyl.

33. The compound of any one of claims 1-25 and 30-32, or a pharmaceutically acceptable salt thereof, wherein R¹ is

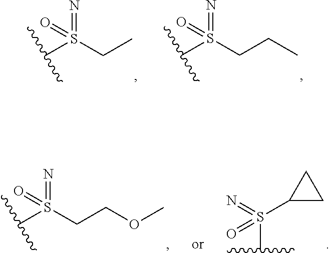

34. The compound of any one of claims 1-25, or a pharmaceutically acceptable salt thereof, wherein
R¹ is 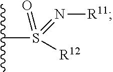
R¹¹ is C₁₋₆ alkyl;
R¹² is C₁₋₆ alkyl, C₁₋₆ haloalkyl, or C₃₋₆ cycloalkyl; the alkyl or cycloalkyl of R¹² is optionally substituted with —OR^{12a}; and
R^{12a} is C₁₋₆ alkyl.

35. The compound of any one of claims 1-25, or a pharmaceutically acceptable salt thereof, wherein
R¹ is 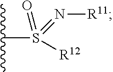
R¹¹ is C₁₋₆ alkyl;
R¹² is C₁₋₆ alkyl or C₃₋₆ cycloalkyl; the alkyl or cycloalkyl of R¹² is optionally substituted with —OR^{12a}; and
R^{12a} is C₁₋₆ alkyl.

36. The compound of any one of claims 1-25, 30, 34, and 35, or a pharmaceutically acceptable salt thereof, wherein R¹ is

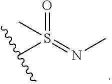 [SMILES: *S(C)(=O)=NC]

37. A compound of Table 1, or a pharmaceutically acceptable salt thereof.

38. A compound of Table 1, or a pharmaceutically acceptable salt thereof, stereoisomer, or mixture of stereoisomers thereof.

39. A pharmaceutical composition comprising a compound of any one of claims 1-38 or a pharmaceutically acceptable salt thereof, and at least one pharmaceutically acceptable carrier.

40. The pharmaceutical composition of claim 39, further comprising one, two, three, or four additional therapeutic agents.

41. The pharmaceutical composition of claim 40, wherein the additional therapeutic agents are selected from famciclovir, acyclovir, and valacyclovir.

42. A method of treating a herpesvirus infection, comprising administering to a patient having a therapeutically effective amount of a compound of any one of claims 1-38 or a pharmaceutically acceptable salt thereof, or a composition of any one of claim 39-41, to a patient in need thereof.

43. The method of claim 42, wherein the method comprises administering a compound of any one of claims 1-38 or a pharmaceutically acceptable salt thereof, or a composition of any one of claim 39-41, in combination with one, two, three, or four additional therapeutic agents.

44. The method of claim 42 or 43, wherein the herpesvirus is HSV-1 or HSV-2.

45. A method of treating a disorder induced, exacerbated, or accelerated by herpesviruses, comprising administering to a patient a therapeutically effective amount of a compound of any one of claims 1-38 or a pharmaceutically acceptable salt thereof, or a composition of any one of claims 39-41, to a patient in need thereof.

46. The method of claim 45, wherein the method comprises administering a compound of any one of claims 1-38 or a pharmaceutically acceptable salt thereof, or a composition of any one of claims 39-41, in combination with one, two, three, or four additional therapeutic agents.

47. The method of claim 45 or 46, wherein the disorder is genital herpes, herpes labialis, HSV keratitis, HSV encephalitis, or disseminated HSV.

48. Use of a compound of any one of claims 1-38, or a pharmaceutically acceptable salt thereof, in the treatment of a viral infection.

49. Use of a compound of any one of claims 1-38, or a pharmaceutically acceptable salt thereof, in the treatment of a viral infection caused by herpesviruses.

50. Use of a compound of any one of claims 1-38, or a pharmaceutically acceptable salt thereof, in the treatment of a viral infection caused by HSV-1 or HSV-2.

51. Use of a compound of any one of claims 1-38, or a pharmaceutically acceptable salt thereof, for the preparation of a medicament for preventing/treating a viral infection.

52. Use of a compound of any one of claims 1-38, or a pharmaceutically acceptable salt thereof, for the preparation of a medicament for preventing/treating a viral infection caused by herpesviruses.

53. Use of a compound of any one of claims 1-38, or a pharmaceutically acceptable salt thereof, for the preparation of a medicament for preventing/treating a viral infection caused by HSV-1 or HSV-2.

54. The compound or pharmaceutically acceptable salt thereof of any one of claims 1-38 or the pharmaceutical composition of any one of claim 39-41 for use in a method of treating a viral infection caused by HSV-1 or HSV-2.

55. The compound, pharmaceutically acceptable salt thereof, or pharmaceutical composition for use of claim 54, wherein the viral infection is genital herpes, herpes labialis, HSV keratitis, HSV encephalitis, or disseminated HSV.

56. The compound, pharmaceutically acceptable salt thereof, or pharmaceutical composition for use of claim 54 or 55, wherein the compound or pharmaceutically acceptable salt thereof is administered in combination with an additional therapeutic agent.

57. The compound or pharmaceutically acceptable salt thereof of any one of claims 1-38 for use in therapy.
